(19) United States
(12) Patent Application Publication
(10) Pub. No.: US 2019/0256608 A1
(43) Pub. Date: Aug. 22, 2019

(54) COMBINATION TREATMENTS AND USES AND METHODS THEREOF

(71) Applicant: GlaxoSmithKline Intellectual Property Development Limited, Brentford, Middlesex (GB)

(72) Inventors: Patrick A. MAYES, Collegville, PA (US); Laura M. SEESTALLER-WEHR, Collegeville, PA (US); Lyuben TSVETKOV, Collegeville, PA (US); Niranjan YANAMANDRA, Collegeville, PA (US); Jingsong YANG, Collegeville, PA (US)

(21) Appl. No.: 15/780,331

(22) PCT Filed: Dec. 1, 2016
(86) PCT No.: PCT/IB2016/057275

§ 371 Date: May 31, 2018

Related U.S. Application Data
(60) Provisional application No. 62/261,481, filed on Dec. 1, 2015.

(51) Int. Cl. C07K 16/28; A61K 47/68; A61P 35/00

(52) U.S. Cl. CPC C07K 16/2878; A61K 47/6803; A61P 35/00; C07K 16/2818; A61K 47/6849

(57) ABSTRACT

Disclosed herein are combinations of an antigen binding protein that binds BCMA with a antigen binding protein that bind to an immunomodulatory agents, such as PD-1 or OX40, pharmaceutical compositions thereof, uses thereof, and methods of treatment comprising administering said combinations, including uses in cancer.

### CROSS-REFERENCE TO RELATED APPLICATIONS

This application claims priority to U.S. Application Ser. No. 62/261,481, filed on Dec. 1, 2015, the disclosure of which is herein incorporated by reference.

### SEQUENCE LISTING

The instant application contains a Sequence Listing which has been submitted electronically in ASCII format and is hereby incorporated by reference in its entirety. Said ASCII copy, created on Nov. 30, 2016, is named PB65987WO_SL.txt and is 293,882 bytes in size.

### FIELD OF THE INVENTION

The present invention relates to combinations comprising anti-BCMA antigen binding proteins, including monoclonal antibodies to human BCMA in combination with immunomodulatory agents such as anti-PD-1 antigen binding agents and/or anti-OX40 antigen binding proteins. Further the invention relates to the use of the combinations in treating cancer in a mammal such as a human.

### BACKGROUND OF THE INVENTION

Effective treatment of hyperproliferative disorders including cancer is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes include abnormalities in signal transduction pathways and response to factors which differ from those found in normal cells.

Immunotherapies are one approach to treat hyperproliferative disorders. A major hurdle that scientists and clinicians have encountered in the development of various types of cancer immunotherapies has been to break tolerance to self antigen (cancer) in order to mount a robust anti-tumor response leading to tumor regression. Unlike traditional development of small and large molecule agents that target the tumor, cancer immunotherapies target cells of the immune system that have the potential to generate a memory pool of effector cells to induce more durable effects and minimize recurrences.

BCMA (CD269 or TNFRSF17) is a member of the TNF receptor superfamily. It is a non-glycosylated integral membrane receptor for the ligands BAFF and APRIL. BCMA's ligands can also bind additional receptors: TACI (Transmembrane Activator and Calcium modulator and cyclophilin ligand Interactor), which binds APRIL and BAFF; as well as BAFF-R (BAFF Receptor or BR3), which shows restricted but high affinity for BAFF. Together, these receptors and their corresponding ligands regulate different embodiments of humoral immunity, B-cell development and homeostasis.

BCMA's expression is typically restricted to the B-cell lineage and is reported to increase in terminal B-cell differentiation. BCMA is expressed by human plasma blasts, plasma cells from tonsils, spleen and bone marrow, but also by tonsillar memory B cells and by germinal centre B cells, which have a TACI-BAFFR low phenotype (Darce et al, 2007). BCMA is virtually absent on naïve and memory B-cells (Novak et al., 2004a and b). The BCMA antigen is expressed on the cell surface so is accessible to the antibody, but is also expressed in the golgi. As suggested by its expression profile, BCMA signalling, typically linked with B-cell survival and proliferation, is important in the late stages of B-cell differentiation, as well as the survival of long lived bone marrow plasma cells (O'Connor et al., 2004) and plasmablasts (Avery et al., 2003). Furthermore, as BCMA binds APRIL with high affinity, the BCMA-APRIL signalling axis is suggested to predominate at the later stages of B-cell differentiation, perhaps being the most physiologically relevant interaction.

Antigen binding proteins and antibodies that bind BCMA and modulate signalling are known in the art and are disclosed as immunotherapy, for example for cancer.

Binding of the PD-1 ligands, PD-L1 and PD-L2, to the PD-1 receptor found on T cells, inhibits T cell proliferation and cytokine production. Upregulation of PD-1 ligands occurs in some tumors and signaling through this pathway can contribute to inhibition of active T-cell immune surveillance of tumors. Antigen binding proteins and antibodies that bind to the PD-1 receptor and block its interaction with PD-L1 and PD-L2 may release PD-1 pathway-mediated inhibition of the immune response, including the anti-tumor immune response.

Enhancing anti-tumor T cell function and inducing T cell proliferation is a powerful and new approach for cancer treatment. Three immune-oncology antibodies (e.g., immuno-modulators) are presently marketed. Anti-CTLA-4 (YERVOY®/ipilimumab) is thought to augment immune responses at the point of T cell priming and anti-PD-1 antibodies (OPDIVO®/nivolumab and KEYTRUDA®/pembrolizumab) are thought to act in the local tumor microenvironment, by relieving an inhibitory checkpoint in tumor specific T cells that have already been primed and activated.

OX40 (e.g. human OX40 (hOX40) or hOX40R) is a tumor necrosis factor receptor family member that is expressed, among other cells, on activated CD4 and CD8 T cells. One of its functions is in the differentiation and long-term survival of these cells. The ligand for OX40 (OX40L) is expressed by activated antigen-presenting cells.

Though there have been many recent advances in the treatment of cancer, there remains a need for more effective and/or enhanced treatment of an individual suffering the effects of cancer. The combinations and methods herein that relate to combining therapeutic approaches for enhancing anti-tumor immunity address this need.

### SUMMARY OF THE INVENTION

The present invention provides combinations comprising a therapeutically effective amount of an antigen binding protein that binds BCMA and a therapeutically effective amount of an antigen binding protein that binds an immunomodulatory target. Examples of immunomodulatory targets include PD-1 and OX40.

In another embodiment, the antigen binding protein that binds BCMA is conjugated to a cytotoxic agent as an immunoconjugate (e.g. an antibody-drug conjugate (ADC)). The cytotoxic agent may include MMAE or MMAF and the cytotoxic agent may be conjugated to the antigen binding protein that binds BCMA via a linker such as citruline-valine or maleimidocaproyl.

In one embodiment, the antigen binding protein that binds BCMA is an antagonist. In another embodiment, the antigen binding protein that binds BCMA is an IgG1 monoclonal antibody.

In one embodiment, the antigen binding protein that binds BCMA is an antibody that comprises CDRH3 of SEQ. ID. NO: 3, CDRH3 variant N99D of SEQ. ID. NO: 200, or variants thereof. In another embodiment, the antigen binding protein that binds BCMA is an antibody that comprises CDR H1 of SEQ. ID. NO: 1, CDRH2 of SEQ. ID. NO: 2, CDRH3 of SEQ. ID. NO: 3 or CDRH3 variant N99D of SEQ. ID. NO: 200, CDRL1 of SEQ. ID. NO: 4, CDRL2 of SEQ. ID. NO: 5, CDRL3 of SEQ. ID. NO: 6 and variants thereof. In yet another embodiment, the antigen binding protein that binds BCMA is an antibody that comprises a heavy chain variable region of SEQ ID NO: 23 and a light chain variable region of SEQ. ID. NO: 31.

The present invention provides combinations comprising a therapeutically effective amount of an antigen binding protein that binds BCMA and a therapeutically effective amount of an antigen binding protein that binds PD-1.

In one embodiment, the antigen binding protein that binds PD-1 is an antagonist. In another embodiment, the antigen binding protein that binds PD-1 is an IgG4 monoclonal antibody.

In one embodiment, the antigen binding protein that binds PD-1 comprises CDR H1 of SEQ. ID. NO: 201, CDRH2 of SEQ. ID. NO: 202, CDRH3 of SEQ. ID. NO: 203, CDRL1 of SEQ. ID. NO: 204, CDRL2 of SEQ. ID. NO: 205, CDRL3 of SEQ. ID. NO: 206, and variants thereof. In yet another embodiment, the antigen binding protein that binds PD-1 comprises a heavy chain variable region of SEQ. ID. NO: 207 and light chain variable region of SEQ. ID. NO: 208.

In yet another embodiment, the antigen binding protein that binds PD-1 is pembrolizumab, nivolumab, or an antibody comprising 90%, 91%, 92%, 93%, 94%, 95%, 96%, 97%, 98%, 99% or 100% sequence homology to either pembrolizumab or nivolumab.

The present invention provides combinations comprising a therapeutically effective amount of an antigen binding protein that binds BCMA and a therapeutically effective amount of an antigen binding protein that binds OX40.

In one embodiment, the antigen binding protein that binds OX40 is an agonist. In another embodiment, the antigen binding protein that binds OX40 is an IgG1 monoclonal antibody.

In one embodiment, the antigen binding protein that binds OX40 comprises CDR H1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, CDRL3 of SEQ. ID. NO: 224, and variants thereof. In yet another embodiment, the antigen binding protein that binds OX40 comprises a heavy chain variable region of SEQ. ID. NO: 229 and a light chain variable region of SEQ. ID. NO: 230.

Also provided are pharmaceutical compositions comprising the combinations of the present invention.

Also provided are methods of treating cancer in a mammal (such as a human) in need thereof comprising administering a therapeutically effective amount of a combination comprising a therapeutically effective amount of an antigen binding protein that binds BCMA and a therapeutically effective amount of at least one antigen binding protein that binds an immunomodulatory target. In one embodiment, the immunomodulatory target is PD1 or OX40. In one embodiment, the cancer is multiple myeloma (MM) or non-Hodgkin's lymphoma B-cell leukemia (NHL). In one embodiment, the antigen binding protein that binds BCMA and the antigen binding that binds PD-1 or OX40 are administered at the same time or sequentially. Methods provide for systemic (e.g. intravenous) or intratumoral administration of the combinations.

Use of the combinations described herein in the treatment of cancer is provided herein.

Use of the combinations described herein in the manufacture of a medicament for the treatment of cancer is contemplated.

Suitably, kits are provided comprising the pharmaceutical compositions of the invention together with one or more pharmaceutically acceptable carriers.

### BRIEF DESCRIPTION OF THE DRAWINGS

FIG. 1. BCMA ADC induces immunogentic cell death in BCMA expressing cancer cell lines

FIG. 1A) Schematic to show Immunogenic cell death (ICD) in BCMA expressing cancer cell lines. ICD is a special type of apoptosis often associated with the cellular release of ATP and HMGB1, and exposure of CRT at the cellular membrane. ICD induces an immune response through engagement of the antigen presentation process by dendritic cells (DCs).

FIG. 1B) aBCMA-MMAF induces ATP, CRT and HMGB1 in the BCMA+ MM cell line

Tested cell lines were treated with Anti-BCMA-MMAF antibody drug conjugate or Mitoxantrone for 48 hours and then assessed for: 1) loss in cell numbers by automated flow cytometry, 2) calreticulin (CRT) exposure was marked with a polyclonal anti-CRT antibody and evaluated by flow cytometry, 3) HMGB1 content in the cellular supernatant was evaluated by ELISA and subsequent absorbance assessment, and 4) ATP in the cellular supernatant was evaluated by subsequent luminescence assessment. Anti-BCMA-MMAF induced ATP release, HMGB1 release and CRT exposure only in NCI-H929, a BCMA positive MM cell line.

FIG. 2: Induction of dendritic cell maturation activation via induction of immunogenetic cell death FIG. 2A) CD83 cell surface expression increased on HLA-DR+ Dendritic cells from three healthy donors following 24 hour co-culture with BCMA ADC treated NCI-H929 Multiple Myeloma cells.

FIG. 2B) CD40 cell surface expression increased on CD11c+ Dendritic cells from three healthy donors following 24 hour co-culture with BCMA ADC treated NCI-H929 Multiple Myeloma cells. Marker expression was measured in (a) untreated (no BCMA ADC) Dendritic cells alone and (b-f) Dendritic cells that were co-cultured with (b) untreated (no BCMA ADC), (c) IgG Control (10 μg/mL), (d) 0.1, (e) 1 and (f) 10 μg/mL BCMA ADC treated (48 hours) NCI-H929 cells. (g) 3 μg/mL Lipopolysaccharide (LPS) treatment of Dendritic cells was included as a positive control. X-axis: Log Mean Fluorescence Intensity (MFI). The vertical line represents the MFI of the IgG control.

FIG. 3. Suppression of IL-10 production from both NCI-H929 cells and the co-culture system with BCMA ADC treated NCI-H929 cells and immature dendritic cells

FIGS. 3A and 3B. IL-10 decreased in the supernatants from co-cultured Dendritic cells from two healthy human donors and BCMA ADC treated NCI-H929 Multiple Myeloma cells. IL-10 was measured in (a) untreated (no BCMA ADC) Dendritic cells alone and (b-f) Dendritic cells that were co-cultured with (b) untreated (no BCMA ADC), (c) IgG Control (10 μg/mL), (d) 0.1, (e) 1 and (f) 10 μg/mL BCMA ADC treated (48 hours) NCI-H929 cells. (g) 3 μg/mL Lipopolysaccharide (LPS) treatment of Dendritic cells is included as a positive control.

FIG. 3C. IL-10 decreased with BCMA ADC treatment in supernatants from NCI-H929 Multiple Myeloma cells cultured alone. IL-10 was measured in Untreated (a), IgG Control (b), 0.1 (c), 1 (d), and 10 μg/mL BCMA ADC (e) treated (48 hours) NCI-H929 Multiple Myeloma cells.

FIG. 4: Effect of BCMA ADC on human T cells FIG. 4A) The average % difference (Avg) and the coefficient of variation (CV) for the percentage (%) and MFI of markers in CD4 cells in PBMC after anti-CD3/anti-CD28 stimulation in the presence of BCMA ADC for 24 and 72 hrs;

FIG. 4B) The average % difference (Avg) and the coefficient of variation (CV) for the percentage (%) and MFI of markers in CD8 cells in PBMC after anti-CD3/anti-CD28 stimulation in the presence of BCMA ADC for 24 and 72 hrs;

FIG. 4C) The average % difference (Avg) and the coefficient of variation (CV) for the percentage (%) CD4 and CD8 cells expressing IFNγ and IL-4 in PBMC with anti-CD3/anti-CD28 stimulation in the presence of BCMA ADC for 48 and 72 hrs;

FIG. 4D) Effect of BCMA ADC on proliferation of CD4+ and CD8+ T cells. CD4+ and CD8+ T cells stimulated with anti-CD3 and anti-CD28 antibodies in the presence or absence of various concentrations of BCMA ADC. After 96 hours cell proliferation was analyzed by flow cytometry.

Data represent mean±SD from 3 different donors.

BCMA-ADC does not appear to have direct effects on human T cells.

FIG. 5: Graphs demonstrating effect of the combination of an anti-BCMA antibody and anti-OX40 antibody on tumor volume (FIG. 5A) and survival rate (FIG. 5B) in EL4-Luc2-hBCMA mice.

FIG. 6: Graphs demonstrating effect of the combination of an anti-BCMA antibody conjugated to MMAF and anti-PD-1 antibody on tumor volume in EL4-Luc2-h BCMA mice.

FIG. 7: Graphs demonstrating effect of the combination of an anti-BCMA antibody conjugated to MMAF and anti-PD-1 antibody on tumor volume in EL4-Luc2-hBCMA mice.

### DETAILED DESCRIPTION OF THE INVENTION

### Combinations

In one embodiment of the invention there is provided a combination comprising a therapeutically effective amount of an antigen binding protein or fragment thereof that binds BCMA and a therapeutically effective amount of an antigen binding protein or fragment thereof that binds an immunomodulatory target.

In one embodiment of the invention there is provided a combination comprising a therapeutically effective amount of an antigen binding protein or fragment thereof that binds BCMA and a therapeutically effective amount of an antigen binding protein of fragment thereof that binds PD-1.

In one embodiment of the invention there is provided a combination comprising a therapeutically effective amount of an antigen binding protein or fragment thereof that binds BCMA and a therapeutically effective amount of an antigen binding protein or fragment thereof that binds OX40.

In one embodiment, there is provided a combination comprising a therapeutically effective amount of an antigen binding protein or fragment thereof that binds BCMA, a therapeutically effective amount of an antigen binding protein of fragment thereof that binds PD-1, and a therapeutically effective amount of an antigen binding protein or fragment thereof that binds OX40.

Antigen Binding Proteins that Bind to BCMA

In one embodiment there is provided antigen binding proteins (e.g. an antibody) or fragments thereof which specifically bind to BCMA, for example which specifically bind human BCMA (hBCMA). In another embodiment, the antigen binding protein that binds BCMA inhibits the binding of BAFF and/or APRIL to the BCMA receptor.

In a further embodiment the antigen binding proteins or fragments thereof have the ability to bind to FcγRIIIA and mediate FcgRIIIA mediated effector functions, or have enhanced FcγRIIIA mediated effector function. In one embodiment of the invention as herein provided the antigen binding proteins are capable of internalization. In one specific embodiment of the invention as herein provided the antigen binding proteins as herein described are capable of internalization at a rapid rate. For example the antigen binding proteins internalize in less than 12 hours, or in less than 6 hours, or in less than 120 minutes. In one embodiment the antigen binding proteins internalize in less than 30 minutes for example in 15 minutes. Internalization of the antigen binding proteins can be measured using techniques known in the art for example by confocal microscopy to visualize the BCMA bound to its receptor and colocalised with intracellular vesicles (endosomes and lysosomes) or present in the cytoplasm or alternatively by flow cytometry to detect the variation over time of the presence of BCMA on the cell surface with disappearance of BCMA indicating internalization.

In a further embodiment the antigen binding proteins of the present invention have effector function for example antibody dependant cellular cytoxicity (ADCC) for example the antigen binding protein has enhanced ADCC effector function.

In a further embodiment the antigen binding proteins are conjugated to a drug which is a cytotoxic agent to form an immunoconjugate (e.g. an antibody-drug conjugate (ADC)). In one such embodiment the cytotoxic agent is an auristatin. In yet a further embodiment the cytotoxic agent is monomethyl auristatin E (MMAE) or monomethyl auristatitin F (MMAF). In one embodiment the immunoconjugate is also ADCC enhanced.

In one embodiment the cytotoxic agent is conjugated to the antigen binding protein that binds BCMA via a linker, such as valine-citruline (VC) or maleimidocaproyl (mc).

In one such embodiment the immunoconjugate is able to cause immunogenic cell death. In one embodiment of the invention there is provided an antigen binding protein according to the invention as herein described which binds to non-membrane bound BCMA, for example to serum BCMA.

In another example, the antigen binding protein that binds to BCMA is an antagonist that blocks binding of BCMA with a BCMA ligand such as BAFF or APRIL.

In one embodiment, the antigen binding protein that binds BCMA contains an immunoglobulin-like domain or fragment thereof. In another embodiment, the antigen binding protein that binds BCMA is a monoclonal antibody, for example IgG, IgM, IgA, IgD or IgE, subclasses thereof, or modified variants thereof. In yet another embodiment, the antigen binding protein that binds BCMA is an IgG antibody.

In one embodiment of the invention there is provided an antigen binding protein as herein described wherein the antigen binding protein comprises CDRH3 of SEQ. ID. NO: 3, CDRH3 variant N99D of SEQ. ID. NO: 200, or variants thereof. In another embodiment, the antigen binding protein comprises a CDRH3 region comprising an amino acid sequence with at least 90%, 91%, 92%, 93%, 94%, 95%, 96%, 97%, 98%, 99% or 100% sequence identity to the amino acid sequence as set forth in SEQ. ID. NO: 3 or SEQ. ID. NO: 200.

In a further embodiment of the invention there is provided an antigen binding protein as herein described wherein the antigen binding protein further comprises one or more of: CDRH1 of SEQ. ID. NO: 1, CDRH2 of SEQ. ID. NO: 2, CDRL1 of SEQ. ID. NO: 4, CDRL2 of SEQ. ID. NO: 5 and/or CDRL3 of SEQ. ID. NO: 6 and or variants thereof.

In one embodiment of the invention there is provided an antigen binding protein as herein described wherein the antigen binding protein comprises CDRH3 of SEQ. ID. NO: 184 or a variant of SEQ. ID. NO: 184.

In a further embodiment of the invention there is provided an antigen binding protein as herein described wherein the antigen binding protein further comprises one or more of: CDRH1 of SEQ. ID. NO: 182, CDRH2 of SEQ. ID. NO: 183, CDRL1 of SEQ. ID. NO: 185, CDRL2 of SEQ. ID. NO: 186 and/or CDRL3 of SEQ. ID. NO: 187 and or variants thereof.

In yet a further embodiment the antigen binding protein comprises CDRH1 of SEQ. ID. NO: 1, CDRH2 of SEQ. ID. NO: 2, CDRH3 variant N99D of SEQ. ID. NO: 200, CDRL1 of SEQ. ID. NO: 4, CDRL2 of SEQ. ID. NO: 5 and CDRL3 of SEQ. ID. NO: 6. In another embodiment, the antigen binding protein comprises CDR regions comprising amino acid sequences with at least 90%, 91%, 92%, 93%, 94%, 95%, 96%, 97%, 98%, 99% or 100% sequence identity to the amino acid sequence as set forth in SEQ. ID. NO: 1, SEQ. ID. NO: 2, SEQ. ID. NO: 200, SEQ. ID. NO: 4, SEQ. ID. NO: 5 and SEQ. ID. NO: 6.

In a further embodiment, the antigen binding protein comprises CDRH1 of SEQ. ID. NO: 1, CDRH2 of SEQ. ID. NO: 2, CDRH3 of SEQ. ID. NO: 3, CDRL1 of SEQ. ID. NO: 4, CDRL2 of SEQ. ID. NO: 5, and CDRL3 of SEQ. ID. NO: 6. In another embodiment, the antigen binding protein comprises CDR regions comprising amino acid sequences with at least 90%, 91%, 92%, 93%, 94%, 95%, 96%, 97%, 98%, 99% or 100% sequence identity to the amino acid sequence as set forth in SEQ. ID. NO: 1, SEQ. ID. NO: 2, SEQ. ID. NO: 3, SEQ. ID. NO: 4, SEQ. ID. NO: 5 and SEQ. ID. NO: 6.

In yet a further embodiment the antigen binding protein comprises CDRH3 of SEQ. ID. NO: 184, CDRH2 of SEQ. ID. NO: 183, CDRH1 of SEQ. ID. NO: 182, CDRL1 of SEQ. ID. NO: 185, CDRL2 of SEQ. ID. NO: 186 and CDRL3 of SEQ. ID. NO: 187.

The antigen binding proteins of the present invention are derived from the murine antibody having the variable regions as described in SEQ. ID. NO: 7 and SEQ. ID. NO: 9 or non-murine equivalents thereof, such as rat, human, chimeric or humanised variants thereof, for example they are derived from the antibody having the variable heavy chain sequences as described in SEQ. ID. NO: 11, SEQ. ID. NO: 13, SEQ. ID. NO: 15, SEQ. ID. NO: 17, SEQ. ID. NO: 19, SEQ. ID. NO: 21, SEQ. ID. NO: 23, SEQ. ID. NO: 25, SEQ. ID. NO: 27 and SEQ. ID. NO: 29 and/or the variable light chain sequences as described in SEQ. ID. NO: 31, SEQ. ID. NO: 33 and/or SEQ. ID. NO: 35.

In another embodiment the antigen binding proteins of the present invention are derived from an antibody having the heavy chain variable region of SEQ ID NO:116 or SEQ ID NO:118 and/or the variable light chain sequences as described in SEQ ID NO:120, or SEQ ID NO:122. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 116 or SEQ ID NO: 118 and/or a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 120, or SEQ. ID. NO: 122.

In another embodiment the antigen binding proteins of the present invention are derived from an antibody having the heavy chain variable region sequences of SEQ. ID. NO: 140 and/or the light chain variable region sequences of SEQ. ID. NO: 144. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 140 and/or a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 144.

In one embodiment of the invention there is provided an antigen binding protein comprising an isolated heavy chain variable region selected from any one of the following: SEQ. ID. NO: 11, SEQ. ID. NO: 13, SEQ. ID. NO: 15, SEQ. ID. NO: 17, SEQ. ID. NO: 19, SEQ. ID. NO: 21, SEQ. ID. NO: 23, SEQ. ID. NO: 25, SEQ. ID. NO: 27, SEQ. ID. NO: 29, SEQ. ID. NO: 116 or SEQ. ID. NO: 118.

In another embodiment of the invention there is provided an antigen binding protein comprising an isolated light chain variable region selected from any one of the following: SEQ. ID. NO: 31, SEQ. ID. NO: 33 or SEQ. ID. NO: 35, SEQ. ID. NO: 120 or SEQ. ID. NO: 122.

In a further embodiment of the invention there is provided an antigen binding protein comprising an isolated heavy chain variable region selected from any one of the following: SEQ. ID. NO: 11, SEQ. ID. NO: 13, SEQ. ID. NO: 15, SEQ. ID. NO: 17, SEQ. ID. NO: 19, SEQ. ID. NO: 21, SEQ. ID. NO: 23, SEQ. ID. NO: 25, SEQ. ID. NO: 27 and SEQ. ID. NO: 29 and an isolated light chain variable region selected from any one of the following: SEQ. ID. NO: 31, SEQ. ID. NO: 33 and/or SEQ. ID. NO: 35.

In one embodiment the antigen binding protein of the present invention comprises a heavy chain variable region of SEQ. ID. NO: 23 and a light chain variable region of SEQ. ID. NO: 31. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 23 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 31.

In one embodiment the antigen binding protein of the present invention comprises a heavy chain variable region of SEQ. ID. NO: 27 and a light chain variable region of SEQ. ID. NO: 31. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 27 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 31.

In one embodiment the antigen binding protein of the present invention comprises a heavy chain variable region of SEQ. ID. NO: 29 and a light chain variable region of SEQ. ID. NO: 31. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 29 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 31.

In one embodiment the antigen binding protein of the present invention comprises a heavy chain variable region of SEQ. ID. NO: 116 and a light chain variable region of SEQ. ID. NO: 120. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 116 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 120.

In one embodiment the antigen binding protein of the present invention comprises a heavy chain variable region of SEQ. ID. NO: 118 and a light chain variable region of SEQ. ID. NO: 122. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 118 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 122.

In one embodiment, the immunoconjugate is GSK2857916. Tai, Blood, 123(2):3128-38 (2014). GSK2857916 comprises an anti-BCMA antibody conjugated to monomethyl auristatitin F (MMAF) via a maleimidocaproyl (mc) linker. GSK2857916 is derived from clone J6M0 and comprises the amino acid sequences of SEQ. ID. NO: 55, SEQ. ID. NO: 63, SEQ. ID. NO: 23, SEQ. ID. NO: 31, SEQ. ID. NO: 1, SEQ. ID. NO: 2, SEQ. ID. NO: 200, SEQ. ID. NO: 4, SEQ. ID. NO: 5, and SEQ. ID. NO: 6.

In one embodiment there is provided a polynucleotide encoding an isolated heavy chain variable region of SEQ. ID. NO: 12, or SEQ. ID. NO: 14, or SEQ. ID. NO: 16, or SEQ. ID. NO: 18, or SEQ. ID. NO: 20, or SEQ. ID. NO: 22, or SEQ. ID. NO: 24, or SEQ. ID. NO: 26, or SEQ. ID. NO: 28, or SEQ. ID. NO: 30 or SEQ. ID. NO: 117 or SEQ. ID. NO: 119 or SEQ. ID. NO: 141.

In one embodiment there is provided a polynucleotide encoding an isolated light chain variable region of SEQ. ID. NO: 32, or SEQ. ID. NO: 34, or SEQ. ID. NO: 36 or SEQ. ID. NO: 121 or SEQ. ID. NO: 123 or SEQ. ID. NO: 145.

In a further embodiment there is provided a polynucleotide encoding an isolated heavy chain variable region of SEQ. ID. NO: 24, or SEQ. ID. NO: 28 or SEQ. ID. NO: 30 and a polynucleotide encoding an isolated light chain variable region of SEQ. ID. NO: 32, or SEQ. ID. NO: 34.

In yet a further embodiment there is provided a polynucleotide encoding an isolated heavy chain variable region of SEQ. ID. NO: 24 and a polynucleotide encoding an isolated light chain variable region of SEQ. ID. NO: 32.

In yet a further embodiment there is provided a polynucleotide encoding an isolated heavy chain variable region of SEQ. ID. NO: 117 and a polynucleotide encoding an isolated light chain variable region of SEQ. ID. NO: 121.

In yet a further embodiment there is provided a polynucleotide encoding an isolated heavy chain variable region of SEQ. ID. NO: 119 and a polynucleotide encoding an isolated light chain variable region of SEQ. ID. NO: 123.

In yet a further embodiment there is provided a polynucleotide encoding an isolated heavy chain variable region of SEQ. ID. NO: 141 and a polynucleotide encoding an isolated light chain variable region of SEQ. ID. NO: 145.

In a further embodiment the antigen binding protein may comprise any one of the heavy chain variable regions as described herein in combination with any one of the light chain variable regions as described herein. In some embodiments, the antigen binding protein can bind (e.g., and antagonize) BCMA, e.g., human BCMA.

In one embodiment the antigen binding protein is an antibody or antigen binding fragment thereof comprising one or more CDR's according to the invention described herein, or one or both of the heavy chain variable region or light chain variable region according to the invention described herein. In one embodiment the antigen binding protein binds primate BCMA. In one such embodiment the antigen binding protein additionally binds non-human primate BCMA, for example cynomolgus macaque monkey BCMA.

In another embodiment the antigen binding protein is selected from the group consisting of a dAb, Fab, Fab′, F(ab′)2, Fv, diabody, triabody, tetrabody, miniantibody, and a minibody.

In one embodiment of the present invention the antigen binding protein is a humanised or chimeric antibody, in a further embodiment the antibody is humanised.

In one embodiment the antibody is a monoclonal antibody.

In one embodiment of the present invention there is provided an antibody with the heavy chain sequence as set forth in SEQ. ID. NO: 55 or SEQ. ID. NO: 59 or SEQ. ID. NO: 61.

In one embodiment of the present invention there is provided an antibody with the light chain sequence as set forth in SEQ. ID. NO: 63 or SEQ. ID. NO: 65.

In a further embodiment of the invention there is provided an antibody with the heavy chain sequence of SEQ. ID. NO: 55 and a light chain sequence as set forth in SEQ. ID. NO: 63. In another embodiment, the antigen binding protein comprises a heavy chain region comprising amino acid sequences with at least 90%, 91%, 92%, 93%, 94%, 95%, 96%, 97%, 98%, 99% or 100% sequence identity to the amino acid sequence as set forth in SEQ. ID. NO: 55 and a light chain region comprising amino acid sequences with at least 90%, 91%, 92%, 93%, 94%, 95%, 96%, 97%, 98%, 99% or 100% sequence identity to the amino acid sequence as set forth in SEQ. ID. NO: 63.

In one embodiment there is provided an antigen binding protein or fragment thereof which competes with an antigen binding protein of the invention as herein described. In one such embodiment there is therefore provided an antigen binding protein which competes with an antigen binding protein which comprises the heavy chain variable sequence of SEQ. ID. NO: 23 and the light chain variable region of SEQ. ID. NO: 31.

In a further embodiment there is therefore provided an antigen binding protein which competes with an antigen binding protein which comprises a heavy chain variable sequence selected from one of SEQ. ID. NO: 27, SEQ. ID. NO: 29, SEQ. ID. NO: 116, SEQ. ID. NO: 118 and SEQ. ID. NO: 140 and a light chain variable region selected from one of SEQ. ID. NO: 31, SEQ. ID. NO: 120, SEQ. ID. NO: 122 and SEQ. ID. NO: 144.

In one embodiment of the invention the antigen binding protein is a chimeric antigen receptor (CAR). In a further embodiment the CAR comprises a binding domain, a transmembrane domain and an intracellular effector domain.

In one embodiment, the transmembrane domain can be derived either from a natural or from a synthetic source. In one embodiment, the transmembrane domain can be derived from any membrane-bound or transmembrane protein. Alternatively the transmembrane domain can be synthetic and can comprise predominantly hydrophobic residues such as leucine and valine. For example, the transmembrane domain can be the transmembrane domain of CD proteins, such as CD4, CD8, CD3 or CD28, a subunit of the T cell receptor, such as α, β, γ or δ, a subunit of the IL-2 receptor (a chain), a submit of the Low-Affinity Nerve Growth Factor Receptor (LNGFR or p75) (β chain or γ chain), or a subunit chain of Fc receptors. In one embodiment, the transmembrane domain comprises the transmembrane domain of CD4, CD8 or CD28. In a further embodiment, the transmembrane domain comprises the transmembrane domain of CD4 or CD8 (e.g. the CD8 alpha chain, as described in NCBI Reference Sequence: NP_001139345.1, incorporated herein by reference). In a yet further embodiment, the transmembrane domain comprises the transmembrane domain of CD4.

The intracellular effector domain or “signalling domain” is responsible for intracellular signalling following the binding of the target binding domain to the target. The intracellular effector domain is responsible for the activation of at least one of the normal effector functions of the immune cell in which the CAR is expressed. For example, the effector function of a T cell can be a cytolytic activity or helper activity including the secretion of cytokines. Preferred examples of the effector domain for use in a CAR scaffold can be the cytoplasmic sequences of the natural T cell receptor and co-receptors that act in concert to initiate signal transduction following antigen binding, as well as any derivate or variant of these sequences and any synthetic sequence that has the same functional capability. Effector domains can be separated into two classes: those that initiate antigen-dependent primary activation, and those that act in an antigen-independent manner to provide a secondary or costimulatory signal. Primary activation effector domains can comprise signalling motifs which are known as immunoreceptor tyrosine-based activation motifs (ITAMs). ITAMs are well defined signalling motifs, commonly found in the intracytoplasmic tail of a variety of receptors, and serve as binding sites for syk/zap70 class tyrosine kinases. Examples of ITAMs used in the invention can include, as non-limiting examples, those derived from CD3zeta, FcRgamma, FcRbeta, FcRepsilon, CD3gamma, CD3delta, CD3epsilon, CD5, CD22, CD79a, CD79b and CD66d. In one embodiment, the intracellular effector domain comprises a CD3zeta signalling domain (also known as CD247). Natural TCRs contain a CD3zeta signalling molecule, therefore the use of this effector domain is closest to the TCR construct which occurs in nature.

In one embodiment of the invention the intracellular signalling domain is a CD3 zeta effector domain.

Effector domains may also provide a secondary or costimulatory signal. T cells additionally comprise costimulatory molecules which bind to cognate costimulatory ligands on antigen presenting cells in order to enhance the T cell response, for example by increasing proliferation activation, differentiation and the like. Therefore, in one embodiment, the intracellular effector domain additionally comprises a costimulatory domain. In a further embodiment, the costimulatory domain comprises the intracellular domain of a costimulatory molecule, selected from CD28, CD27, 4-1BB (CD137), OX40 (CD134), ICOS (CD278), CD30, CD40, PD-1 (CD279), CD2, CD7, NKG2C (CD94), B7-H3 (CD276) or any combination thereof. In a yet further embodiment, the costimulatory domain comprises the intracellular domain of a costimulatory molecule, selected from CD28, CD27, 4-1BB, OX40, ICOS or any combination thereof.

Competition between an antigen binding protein of the present invention and a reference antibody may be determined by competition ELISA, FMAT or Biacore. In one embodiment, the competition assay is carried out by Biacore. There are several possible reasons for this competition: the two proteins may bind to the same or overlapping epitopes, there may be steric inhibition of binding, or binding of the first protein may induce a conformational change in the antigen that prevents or reduces binding of the second protein.

In another embodiment the antigen binding protein binds to human BCMA with high affinity for example when measured by Biacore the antigen binding protein binds to human BCMA with an affinity of 20 nM or less or an affinity of 15 nM or less or an affinity of 5 nM or less or an affinity of 1000 pM or less or an affinity of 500 pM or less or an affinity of 400 pM or less, or 300 pM or less or for example about 120 pM. In a further embodiment the antigen binding protein binds to human BCMA when measured by Biacore of between about 100 pM and about 500 pM or between about 100 pM and about 400 pM, or between about 100 pM and about 300 pM. In one embodiment of the present invention the antigen binding protein binds BCMA with an affinity of less than 150 pm.

In one such embodiment, this is measured by Biacore, for example as set out in Example 4 of WO2012163805 as herein incorporated by reference.

In another embodiment the antigen binding protein binds to human BCMA and neutralizes the binding of the ligands BAFF and/or APRIL to the BCMA receptor in a cell neutralization assay wherein the antigen binding protein has an IC50 of between about 1 nM and about 500 nM, or between about 1 nM and about 100 nM, or between about 1 nM and about 50 nM, or between about 1 nM and about 25 nM, or between about 5 nM and about 15 nM. In a further embodiment of the present invention the antigen binding protein binds BCMA and neutralizes BCMA in a cell neutralization assay wherein the antigen binding protein has an IC50 of about 10 nM.

In one such embodiment, this is measured by a cell neutralization assay, for example as set out in Example 4.6 of WO2012163805 as herein incorporated by reference.

Antigen Binding Proteins that Bind to Pd-1

In one embodiment of the invention, as herein described, the combination comprises an antigen binding protein (e.g. an antibody) which specifically binds to BCMA and an antigen binding protein (e.g. an antibody) or fragment thereof which specifically binds to PD-1. In one example, the antigen binding protein that binds to PD-1 specifically binds human PD-1 (hPD-1). In another example, the antigen binding protein that binds to PD-1 is an antagonist that blocks binding of PD-1 with a PD-1 ligand such as PD-L1 or PD-L2.

In one embodiment, the antigen binding protein that binds PD-1 contains an immunoglobulin-like domain or fragment thereof. In another embodiment, the antigen binding protein that binds PD-1 is a monoclonal antibody, for example IgG, IgM, IgA, IgD or IgE, subclasses thereof, or modified variants thereof. In yet another embodiment, the antigen binding protein that binds PD-1 is an IgG antibody. In another embodiment, the antigen binding protein that binds PD-1 is an IgG4 antibody.

In one embodiment of the invention there is provided an antigen binding protein as herein described wherein the antigen binding protein comprises CDRH3 of SEQ. ID. NO: 203 or a variant thereof. In another embodiment, the antigen binding protein comprises a CDRH3 region comprising an amino acid sequence with at least 90%, 91%, 92%, 93%, 94%, 95%, 96%, 97%, 98%, 99% or 100% sequence identity to the amino acid sequence as set forth in SEQ. ID. NO: 203.

In a further embodiment of the invention there is provided an antigen binding protein as herein described wherein the antigen binding protein comprises CDRH1 of SEQ. ID. NO: 201, CDRH2 of SEQ. ID. NO: 202, CDRH3 of SEQ. ID. NO: 203, CDRL1 of SEQ. ID. NO: 204, CDRL2 of SEQ. ID. NO: 205, CDRL3: SEQ. ID. NO: 206, and or variants thereof. In another embodiment, the antigen binding protein comprises CDR regions comprising amino acid sequences with at least 90%, 91%, 92%, 93%, 94%, 95%, 96%, 97%, 98%, 99% or 100% sequence identity to the amino acid sequences as set forth in SEQ. ID. NO: 201, SEQ. ID. NO: 202, SEQ. ID. NO: 203, SEQ. ID. NO: 204, SEQ. ID. NO: 205 and SEQ. ID. NO: 206.

In one embodiment of the invention there is provided an antigen binding protein as herein described wherein the antigen binding protein comprises CDRH3 of SEQ. ID. NO: 213 or a variant thereof.

In a further embodiment of the invention there is provided an antigen binding protein as herein described wherein the antigen binding protein further comprises one or more of: CDRH1 of SEQ. ID. NO: 211, CDRH2 of SEQ. ID. NO: 212, CDRL1 of SEQ. ID. NO: 214, CDRL2 of SEQ. ID. NO: 215 and/or CDRL3 of SEQ. ID. NO: 216 or variants thereof.

In yet a further embodiment the antigen binding protein comprises CDRH3 of SEQ. ID. NO: 203, CDRH2 of SEQ. ID. NO: 202, CDRH1 of SEQ. ID. NO: 201, CDRL1 of SEQ. ID. NO: 204, CDRL2 of SEQ. ID. NO: 205 and CDRL3 of SEQ. ID. NO: 206.

In yet a further embodiment the antigen binding protein comprises CDRH3 of SEQ. ID. NO: 213, CDRH2 of SEQ. ID. NO: 211, CDRH1 of SEQ. ID. NO: 212, CDRL1 of SEQ. ID. NO: 214, CDRL2 of SEQ. ID. NO: 215 and CDRL3 of SEQ. ID. NO: 216.

In one embodiment of the invention there is provided an antigen binding protein comprising an isolated heavy chain variable domain selected from SEQ. ID. NO: 207 or SEQ. ID. NO: 217. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 207 or SEQ. ID. NO: 217.

In another embodiment of the invention there is provided an antigen binding protein comprising an isolated light chain variable region selected from SEQ. ID. NO: 208 or SEQ. ID. NO: 218. In another embodiment, the antigen binding protein comprises a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 208 or SEQ. ID. NO: 218.

In a further embodiment of the invention there is provided an antigen binding protein comprising an isolated heavy chain variable region selected from SEQ. ID. NO: 207 or SEQ. ID. NO: 217 and an isolated light chain variable domain selected from SEQ. ID. NO: 208 or SEQ. ID. NO: 218. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 207 or SEQ. ID. NO: 217 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 208 or SEQ. ID. NO: 218.

In one embodiment the antigen binding protein of the present invention comprises a heavy chain variable region encoded by SEQ. ID. NO: 207 and a light chain variable region encoded by SEQ. ID. NO: 208. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 207 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 208.

In one embodiment the antigen binding protein of the present invention comprises a heavy chain variable region encoded by SEQ. ID. NO: 217 and a light chain variable region encoded by SEQ. ID. NO: 218. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 217 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 218.

In one combination contemplated, the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ ID.NO: 200, CDRL1 of SEQ. ID. NO: 4, CDRL2 of SEQ. ID. NO: 5, CDRL3 of SEQ. ID. NO: 6 or variants thereof conjugated to MMAF; and the antigen binding protein that binds PD-1 comprises CDRH1 of SEQ. ID. NO: 201, CDRH2 of SEQ. ID. NO: 202, CDRH3 of SEQ ID NO: 203, CDRL1 of SEQ. ID. NO: 204, CDRL2 of SEQ. ID. NO: 205, CDRL3 of SEQ. ID. NO: 206, or variants thereof.

In one embodiment there is provided a polynucleotide encoding an isolated heavy chain variable region of SEQ. ID. NO: 207. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 207.

In one embodiment there is provided a polynucleotide encoding the CDR regions of SEQ. ID. NO: 201, SEQ. ID. NO: 202, SEQ. ID. NO: 203, SEQ. ID. NO: 204, SEQ. ID. NO: 205 and SEQ. ID. NO: 206. In another embodiment, the antigen binding protein comprises CDR regions comprising amino acid sequences with at least 90%, 91%, 92%, 93%, 94%, 95%, 96%, 97%, 98%, 99% or 100% sequence identity to the amino acid sequence as set forth in SEQ. ID. NO: 201, SEQ. ID. NO: 202, SEQ. ID. NO: 203, SEQ. ID. NO: 204, SEQ. ID. NO: 205 and SEQ. ID. NO: 206.

In one embodiment the antigen binding protein is an antibody or antigen binding fragment thereof comprising one or more CDR's according to the invention described herein, or one or both of the heavy chain variable regions or light chain variable regions according to the invention described herein.

In another embodiment the antigen binding protein is selected from the group consisting of a dAb, Fab, Fab′, F(ab′)₂, Fv, diabody, triabody, tetrabody, miniantibody, and a minibody.

In one embodiment the antigen binding proteins of the present invention are humanised or chimeric antibodies, in a further embodiment the antibody is humanised.

In one embodiment the antibody is a monoclonal antibody.

In one embodiment of the present invention there is provided an antibody with the heavy chain sequence as set forth in SEQ. ID. NO: 209.

In one embodiment of the present invention there is provided an antibody with the light chain sequence as set forth in SEQ. ID. NO: 210.

In a further embodiment of the invention there is provided an antibody with the heavy chain sequence of SEQ. ID. NO: 209 and a light chain sequence as set forth in SEQ ID. NO: 210.

In one embodiment there is provided an antigen binding protein or fragment thereof which competes with an antigen binding protein of the invention as herein described. In one such embodiment there is therefore provided an antigen binding protein which competes with an antigen binding protein which comprises the heavy chain variable sequence of SEQ. ID. NO: 207 and the light chain variable region of SEQ. ID. NO: 208.

The isolated antibodies as described herein bind to human PD-1, and may bind to human PD-1 encoded by the gene Pdcd1, or genes or cDNA sequences having 90 percent homology or 90 percent identity thereto. The complete hPD-1 mRNA sequence can be found under GenBank Accession No. U64863. The protein sequence for human PD-1 can be found at GenBank Accession No. AAC51773.

In another embodiment the antigen binding protein binds to human PD-1 with high affinity for example when measured by Biacore the antigen binding protein binds to human PD-1 with an affinity of 1 nM or less. In one embodiment of the present invention the antigen binding protein binds PD-1 with an affinity of less than 100 pm.

The binding affinity of pembrolizumab for cynomolgus PD-1 was evaluated by ELISA, cellular ELISA and by bio-light interferometry. In these studies, the binding affinity of pembrolizumab to cynomolgus and human PD-1 was found to be in the same range, albeit slightly lower for cynomolgus PD-1. By kinetic analysis, KD was 29 pM for human PD-1 and 118 pM for cynomolgus PD-1. Functionally, pembrolizumab blocked the binding of human PD-1 ligands to cells expressing human or cynomolgus PD-1 with a comparable potency. A skilled person will appreciate that the smaller the KD numerical value, the stronger the binding. The reciprocal of KD (i.e. 1/KD) is the equilibrium association constant (KA) having units M-1. A skilled person will appreciate that the larger the KA numerical value, the stronger the binding.

The dissociation rate constant (kd) or “off-rate” describes the stability of the complex i.e. the fraction of complexes that decay per second. For example, a kd of 0.01 s−1 equates to 1% of the complexes decaying per second. In an embodiment, the dissociation rate constant (kd) is 1×10−3 s−1 or less, 1×10−4 s−1 or less, 1×10−5 s−1 or less, or 1×10−6 s−1 or less. The kd may be between 1×10−5 s−1 and 1×10−4 s−1; or between 1×10−4 s−1 and 1×10−3 s−1.

Competition between an antigen binding protein of the present invention and a reference antibody may be determined by competition ELISA, FMAT or Biacore. In one embodiment, the competition assay is carried out by Biacore. There are several possible reasons for this competition: the two proteins may bind to the same or overlapping epitopes, there may be steric inhibition of binding, or binding of the first protein may induce a conformational change in the antigen that prevents or reduces binding of the second protein.

In one embodiment of the invention the PD-1 antigen binding protein is Pembrolizumab also known as KEYTRUDA® or MK3475 and as lambrolizumab. Pembrolizumab is a monoclonal antibody that binds to the PD-1 receptor and blocks its interaction with PD-L1 and PD-L2, releasing PD-1 pathway-mediated inhibition of the immune response, including the anti-tumor immune response.

In syngeneic mouse tumor models, blocking PD-1 activity resulted in decreased tumor growth. Pembrolizumab is an IgG4 kappa immunoglobulin with an approximate molecular weight of 149 kDa.

Pembrolizumab (KEYTRUDA®) is a human programmed death receptor-1 (PD-1)-blocking antibody indicated for the treatment of patients with unresectable or metastatic melanoma and disease progression following ipilimumab and, if BRAF V600 mutation positive, a BRAF inhibitor. The recommended dose of pembrolizumab is 2 mg/kg administered as an intravenous infusion over 30 minutes every 3 weeks until disease progression or unacceptable toxicity.

Pembrolizumab is a sterile, preservative-free, white to off-white lyophilized powder in single-use vials. Each vial is reconstituted and diluted for intravenous infusion. Each 2 mL of reconstituted solution contains 50 mg of pembrolizumab and is formulated in L-histidine (3.1 mg), polysorbate-80 (0.4 mg), sucrose (140 mg). May contain hydrochloric acid/sodium hydroxide to adjust pH to 5.5.

Pembrolizumab is described, e.g. in U.S. Pat. Nos. 8,354,509 and 8,900,587.

Pembrolizumab has been approved for the treatment of patients with unresectable or metastatic melanoma and disease progression following ipilimumab and, if BRAF V600 mutation positive, a BRAF inhibitor.

In one embodiment, the combination comprises pembrolizumab and GSK2857916.

As another example, an anti-BCMA antibody can be used in combination with nivolumab) (OPDIVO®). Nivolumab is a human immunoglobulin G4 (IgG4) monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

Nivolumab (OPDIVO®) is a programmed death receptor-1 (PD-1) blocking antibody indicated for the treatment of patients with: unresectable or metastatic melanoma and disease progression following ipilimumab and, if BRAF V600 mutation positive, a BRAF inhibitor. This indication is approved under accelerated approval based on tumor response rate and durability of response. Continued approval for this indication may be contingent upon verification and description of clinical benefit in the confirmatory trials and metastatic squamous non-small cell lung cancer with progression on or after platinum-based chemotherapy.

The recommended dose of nivolumab (OPDIVO®) is 3 mg/kg administered as an intravenous infusion over 60 minutes every 2 weeks until disease progression or unacceptable toxicity.

U.S. Pat. No. 8,008,449 exemplifies seven anti-PD-1 HuMAbs: 17D8, 2D3, 4H1, 5C4 (also referred to herein as nivolumab or BMS-936558), 4A1 1, 7D3 and 5F4. See also U.S. Pat. No. 8,779,105. Any one of these antibodies, or the CDRs thereof (or an amino acid sequence with at least 90% (e.g., 90, 91, 92, 93, 94, 95, 96, 97, 98, or 99%) identity to any of these amino acid sequences), can be used in the compositions and methods described herein.
Antigen Binding Proteins that Bind to OX40

In one embodiment of the invention, as herein described, the combination comprises an antigen binding protein (e.g. an antibody) which specifically binds to BCMA and an antigen binding protein (e.g. an antibody) or fragment thereof which specifically binds to OX40. In one example, the antigen binding protein that binds to OX40 specifically binds human OX40 (hOX40). In another example, the antigen binding protein that binds to OX40 is an agonist.

In one embodiment, the antigen binding protein that binds OX40 contains an immunoglobulin-like domain or fragment thereof. In another embodiment, the antigen binding protein that binds OX40 is a monoclonal antibody, for example IgG, IgM, IgA, IgD or IgE, subclasses thereof, or modified variants thereof. In yet another embodiment, the antigen binding protein that binds OX40 is an IgG1 antibody.

In one embodiment of the invention there is provided an antigen binding protein as herein described wherein the antigen binding protein comprises CDRH3 of SEQ. ID. NO: 221 or a variant thereof. In another embodiment, the antigen binding protein comprises a CDRH3 region comprising an amino acid sequence with at least 90%, 91%, 92%, 93%, 94%, 95%, 96%, 97%, 98%, 99% or 100% sequence identity to the amino acid sequence as set forth in SEQ. ID. NO: 221.

In a further embodiment of the invention there is provided an antigen binding protein as herein described wherein the antigen binding protein comprises CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223 and CDRL3 of SEQ. ID. NO: 224 or variants thereof. In another embodiment, the antigen binding protein comprises CDR regions comprising amino acid sequences with at least 90%, 91%, 92%, 93%, 94%, 95%, 96%, 97%, 98%, 99% or 100% sequence identity to the amino acid sequence as set forth in SEQ. ID. NO: 219, SEQ. ID. NO: 220, SEQ. ID. NO: 221, SEQ. ID. NO: 222, SEQ. ID. NO: 223 and SEQ. ID. NO: 224 In one embodiment of the invention there is provided an antigen binding protein as herein described wherein the antigen binding protein comprises CDRH3 of SEQ. ID. NO: 233 or a variant thereof.

In a further embodiment of the invention there is provided an antigen binding protein as herein described wherein the antigen binding protein further comprises one or more of: CDRH1 of SEQ. ID. NO: 231, CDRH2 of SEQ. ID. NO: 232, CDRL1 of SEQ. ID. NO: 234, CDRL2 of SEQ. ID. NO: 235 and/or CDRL3 of SEQ. ID. NO: 236 and or variants thereof.

In one embodiment of the invention there is provided an antigen binding protein comprising an isolated heavy chain variable region selected from: SEQ ID NO: 229, SEQ ID NO: 225, or SEQ ID NO: 237.

In another embodiment of the invention there is provided an antigen binding protein comprising an isolated light chain variable region selected from SEQ. ID. NO: 230, SEQ. ID. NO: 227, or SEQ. ID. NO: 239.

In a further embodiment of the invention there is provided an antigen binding protein comprising an isolated heavy chain variable region selected from: SEQ. ID. NO: 225 or SEQ. ID. NO: 237 and an isolated light chain variable region selected from: SEQ. ID. NO: 227 or SEQ. ID. NO: 239.

In one embodiment the antigen binding protein of the present invention comprises a heavy chain variable region of SEQ. ID. NO: 225 and a light chain variable region of SEQ. ID. NO: 227. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 225 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 227.

In one embodiment the antigen binding protein of the present invention comprises a heavy chain variable region of SEQ. ID. NO: 237 and a light chain variable region of SEQ. ID. NO: 239. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 237 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 239.

In a further embodiment of the invention there is provided an antigen binding protein comprising an isolated heavy chain variable region of SEQ. ID. NO: 229 and an isolated light chain variable region of SEQ. ID. NO: 230. In another embodiment, the antigen binding protein comprises a heavy chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 229 and a light chain variable region comprising amino acid sequences with at least 90%, 91%, 92%, 93%, 94%, 95%, 96%, 97%, 98%, 99% or 100% sequence identity to the amino acid sequence as set forth in SEQ. ID. NO: 230.

In one embodiment of the invention there is provided an antigen binding protein comprising an isolated heavy chain region of SEQ. ID. NO: 243 and an isolated light chain region of SEQ. ID. NO: 244. In another embodiment, the antigen binding protein comprises a heavy chain region comprising amino acid sequences with at least 90%, 91%, 92%, 93%, 94%, 95%, 96%, 97%, 98%, 99% or 100% sequence identity to the amino acid sequence as set forth in SEQ. ID. NO: 243 and a light chain region comprising amino acid sequences with at least 90%, 91%, 92%, 93%, 94%, 95%, 96%, 97%, 98%, 99% or 100% sequence identity to the amino acid sequence as set forth in SEQ. ID. NO: 244.

In one combination contemplated, the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ ID. NO: 200, CDRL1 of SEQ. ID. NO: 4, CDRL2 of SEQ. ID. NO: 5, CDRL3 of SEQ. ID. NO: 6 or variants thereof conjugated to MMAF; and the antigen binding protein that binds OX40 comprises CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223 and CDRL3 of SEQ. ID. NO: 224 or variants thereof.

In one embodiment there is provided a polynucleotide encoding an isolated heavy chain variable region of SEQ. ID. NO: 229, SEQ. ID. NO: 226, or SEQ. ID. NO: 238.

In one embodiment there is provided a polynucleotide encoding an isolated light chain variable region comprising SEQ. ID. NO: 230, SEQ. ID. NO: 228, or SEQ. ID. NO: 240.

In a further embodiment there is provided a polynucleotide encoding an isolated heavy chain variable region of SEQ. ID. NO: 229, SEQ. ID. NO: 226, or SEQ. ID. NO: 238 and a polynucleotide encoding an isolated light chain variable region of SEQ. ID. NO: 230, SEQ. ID. NO: 228, or SEQ. ID. NO: 240.

In one embodiment, the antigen binding protein of the invention binds its target (e.g. OX40) with high affinity. For example, when measured by Biacore, the antibody binds to OX40, preferably human OX40, with an affinity of 1-1000 nM or 500 nM or less or an affinity of 200 nM or less or an affinity of 100 nM or less or an affinity of 50 nM or less or an affinity of 500 pM or less or an affinity of 400 pM or less, or 300 pM or less. In a further embodiment the antibody binds to OX40, preferably human OX40, when measured by Biacore of between about 50 nM and about 200 nM or between about 50 nM and about 150 nM. In one embodiment of the present invention the antibody binds OX40, preferably human OX40, with an affinity of less than 100 nM.

Competition between an antigen binding protein of the present invention and a reference antibody may be determined by competition ELISA, FMAT or Biacore. In one embodiment, the competition assay is carried out by Biacore. There are several possible reasons for this competition: the two proteins may bind to the same or overlapping epitopes, there may be steric inhibition of binding, or binding of the first protein may induce a conformational change in the antigen that prevents or reduces binding of the second protein.

In an embodiment, the equilibrium dissociation constant (KD) of the antigen binding protein of a combination of the invention, or a method or use thereof, and OX40, preferably human OX40, interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding.

In a further embodiment the antigen binding protein may comprise any one of the variable heavy chains as described herein in combination with any one of the light chains as described in Table A herein.

In one embodiment, the antigen binding protein is a monoclonal antibody that binds to OX40 and is administered at a dose of about 0.1 mg/kg to about 10 mg/kg.

In one embodiment, the antigen binding protein is a monoclonal antibody that binds to OX40 and is administered at a dose selected from the group consisting of: about 0.003 mg/kg, about 0.01 mg/kg, about 0.03 mg/kg, about 0.1 mg/kg, about 0.3 mg/kg, about 1 mg/kg, about 3 mg/kg, and about 10 mg/kg.

In another embodiment, the monoclonal antibody that binds to OX40 is administered at a frequency selected from the group consisting of: once daily, once weekly, once every two weeks (Q2W), and once every three weeks (Q3W).

In yet another embodiment, the monoclonal antibody that binds to human PD-1 is administered at a dose of about 200 mg.

In one embodiment, the monoclonal antibody that binds to OX40 is administered at a dose of about 0.1 mg/kg to about 10 mg/kg. The monoclonal antibody that binds to OX40 can be administered at a frequency selected from: once daily, once weekly, once every two weeks (Q2W) and once every three weeks (Q3W). Suitably, the antibody that binds to OX40 is administered once every three weeks. In one aspect, the pembrolizumab is administered at a dose of 200 mg Q3W.

### Pharmaceutical Compositions and Methods of Treatment

The invention further provides pharmaceutical compositions, which include the combinations described herein, and one or more pharmaceutically acceptable carriers, diluents, or excipients. The combination of the invention may comprise two pharmaceutical compositions, one comprising an anti-BCMA antigen binding proteins or fragments, and the other comprising an anti-PD-1 or OX40 antigen binding protein or fragments thereof, each of which may have the same or different carriers, diluents or excipients. The carrier(s), diluent(s) or excipient(s) must be acceptable in the sense of being compatible with the other ingredients of the formulation, capable of pharmaceutical formulation, and not deleterious to the recipient thereof.

The components of the combination of the invention, and pharmaceutical compositions comprising such components may be administered sequentially in any order, and in different routes; the components and pharmaceutical compositions comprising the same may be administered simultaneously.

Each of the components of the combinations described herein can be manufactured into the same vial/container or into different vials/containers. For example, in one embodiment, an antigen binding protein that binds BCMA is manufactured into the same vial/container as an antigen binding protein that binds to PD-1 or an antigen binding protein that binds to OX40 and all components of the combination are administered simultaneously. In another exemplary embodiment, an antigen binding protein that binds BCMA is manufactured into a different vial/container as an antigen binding protein that binds to PD-1 or an antigen binding protein that binds to OX40 and each of the components of the combination can be administered simultaneously, sequentially, and with the same or different routes of administration.

In accordance with another embodiment of the invention there is also provided a process for the preparation of a pharmaceutical composition including admixing a component of the combination of the invention and one or more pharmaceutically acceptable carriers, diluents or excipients.

The components of the invention may be administered by any appropriate route. For some components, suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that the components may be compounded together or in separate pharmaceutical compositions.

In one embodiment, one or more components of a combination of the invention are administered intravenously. In another embodiment, one or more components of a combination of the invention are administered intratumorally. In another embodiment, one or more components of a combination of the invention are administered systemically, e.g. intravenously, and one or more other components of a combination of the invention are administered intratumorally. In another embodiment, all of the components of a combination of the invention are administered systemically, e.g. intravenously. In an alternative embodiment, all of the components of the combination of the invention are administered intratumorally. In any of the embodiments, e.g. in this paragraph, the components of the invention are administered as one or more pharmaceutical compositions.

The administration of a therapeutically effective amount of the combinations of the invention (or therapeutically effective amounts of each of the components of the combination) are advantageous over the individual component compounds in that the combinations provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

In one embodiment, the present invention provides methods of treating cancer such as in treatment of B cell disorders in a mammal (e.g. a human) in need thereof comprising administering a therapeutically effective amount of the combination as herein described. In one embodiment the cancer is multiple myeloma. In another embodiment the cancer is non-Hodgkin's lymphoma.

In one embodiment, methods are provided for treating cancer comprising administering any one of the antigen binding proteins that bind BCMA of the present invention or antibody drug conjugates thereof; and pembrolizumab, or an antibody comprising 90%, 91%, 92%, 93%, 94%, 95%, 96%, 97%, 98%, 99% or 100% sequence homology thereto.

In one embodiment, methods are provided for treating cancer comprising administering any one of the antigen binding proteins that bind BCMA or immunoconjugates thereof; and nivolumab, or an antibody comprising 90%, 91%, 92%, 93%, 94%, 95%, 96%, 97%, 98%, 99% or 100% sequence homology thereto

In one embodiment, methods are provided for treating cancer in a human in need thereof comprising administering a therapeutically effective amount of a combination comprising:
i) a therapeutically effective amount an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount an antigen binding protein that binds PD-1 comprising CDRH1 of SEQ. ID. NO: 201, CDRH2 of SEQ. ID. NO: 202, CDRH3 of SEQ. ID. NO: 203, CDRL1 of SEQ. ID. NO: 204, CDRL2 of SEQ. ID. NO: 205, CDRL3 of SEQ. ID. NO: 206, or variants thereof.

In one embodiment, methods are provided for treating cancer in a human in need thereof comprising administering a therapeutically effective amount of a combination comprising:
i) a therapeutically effective amount an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of pembrolizumab or nivolumab.

In one embodiment, methods are provided for treating cancer in a human in need thereof comprising administering a therapeutically effective amount of a combination comprising:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds OX40 comprising CDRH1 of SEQ. ID. NO: 219, CDRH2 of SEQ. ID. NO: 220, CDRH3 of SEQ ID NO: 221, CDRL1 of SEQ. ID. NO: 222, CDRL2 of SEQ. ID. NO: 223, CDRL3 of SEQ. ID. NO: 224, or variants thereof.

In one embodiment, methods are provided for treating multiple myeloma in a human in need thereof comprising administering a therapeutically effective amount of a combination comprising:
i) a therapeutically effective amount an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount an antigen binding protein that binds PD-1 comprising CDRH1 of SEQ. ID. NO: 201, CDRH2 of SEQ. ID. NO: 202, CDRH3 of SEQ. ID NO: 203, CDRL1 of SEQ. ID. NO: 204, CDRL2 of SEQ. ID. NO: 205, CDRL3 of SEQ. ID. NO: 206, or variants thereof.

In one embodiment, methods are provided for treating non-Hodgkins lymphoma in a human in need thereof comprising administering a therapeutically effective amount of a combination comprising:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds PD-1 comprising CDRH1 of SEQ. ID. NO: 201, CDRH2 of SEQ. ID. NO: 202, CDRH3 of SEQ. ID NO: 203, CDRL1 of SEQ. ID. NO: 204, CDRL2 of SEQ. ID. NO: 205, CDRL3 of SEQ. ID. NO: 206, or variants thereof.

In one embodiment, methods are provided for treating multiple myeloma in a human in need thereof comprising administering a therapeutically effective amount of a combination comprising:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds OX40 comprising CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, CDRL3 of SEQ. ID. NO: 224, or variants thereof.

In one embodiment, methods are provided for treating non-Hodgkins lymphoma in a human in need thereof comprising administering a therapeutically effective amount of a combination comprising:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds OX40 comprising CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, CDRL3 of SEQ. ID. NO: 224, or variants thereof.

Methods are provided wherein the tumor size of the cancer in said mammal is reduced by more than an additive amount compared with treatment with the antigen binding protein to BCMA and the antigen binding protein that binds PD-1 or the antigen binding protein that binds OX40 as used as monotherapy. Suitably the combination may be synergistic.

In one embodiment, the mammal has increased survival when treated with the combination as herein described compared with a mammal who received the antigen binding protein to BCMA or the antigen binding protein to PD-1 or OX40 as a monotherapy. In one embodiment, the methods further comprise administering at least one anti-neoplastic agent to the mammal in need thereof.

Use in the treatment of cancer, in particular in the treatment of B cell disorders in a mammal (e.g. a human) in need thereof, is contemplated. In one embodiment the cancer is multiple myeloma. In another embodiment the cancer is non-Hodgkin's lymphoma.

In one embodiment, use of the combinations described herein is provided for treating cancer wherein the combination comprises any one of the antigen binding proteins that bind BCMA of the present invention or antibody drug conjugates thereof; and pembrolizumab, or an antibody comprising 90%, 91%, 92%, 93%, 94%, 95%, 96%, 97%, 98%, 99% or 100% sequence homology thereto.

In one embodiment, use of the combinations described herein is provided for treating cancer wherein the combination comprises any one of the antigen binding proteins that bind BCMA or immunoconjugates thereof; and nivolumab, or an antibody comprising 90%, 91%, 92%, 93%, 94%, 95%, 96%, 97%, 98%, 99% or 100% sequence homology thereto

In one embodiment, use of combinations is provided for treating cancer in a human in need thereof wherein the combination comprises:
i) a therapeutically effective amount an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount an antigen binding protein that binds PD-1 comprising CDRH1 of SEQ. ID. NO: 201, CDRH2 of SEQ. ID. NO: 202, CDRH3 of SEQ. ID. NO: 203, CDRL1 of SEQ. ID. NO: 204, CDRL2 of SEQ. ID. NO: 205, CDRL3 of SEQ. ID. NO: 206, or variants thereof.

In one embodiment, use of combinations is provided for treating cancer in a human in need thereof wherein the combination comprises:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds OX40 comprising CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, CDRL3 of SEQ. ID. NO: 224, or variants thereof.

In one embodiment, use of combinations is provided for treating multiple myeloma in a human in need thereof wherein the combination comprises:
i) a therapeutically effective amount an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount an antigen binding protein that binds PD-1 comprising CDRH1 of SEQ. ID. NO: 201, CDRH2 of SEQ. ID. NO: 202, CDRH3 of SEQ ID. NO: 203, CDRL1 of SEQ. ID. NO: 204, CDRL2 of SEQ. ID. NO: 205, CDRL3 of SEQ. ID. NO: 206, or variants thereof.

In one embodiment, use of combinations is provided for treating non-Hodgkin's lymphoma in a human in need thereof wherein the combination comprises:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds PD-1 comprising CDRH1 of SEQ. ID. NO: 201, CDRH2 of SEQ. ID. NO: 202, CDRH3 of SEQ. ID. NO: 203, CDRL1 of SEQ. ID. NO: 204, CDRL2 of SEQ. ID. NO: 205, CDRL3 of SEQ. ID. NO: 206, or variants thereof.

In one embodiment, use of combinations is provided for treating multiple myeloma in a human in need thereof wherein the combination comprises:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds OX40 comprising CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, CDRL3 of SEQ. ID. NO: 224, or variants thereof.

In one embodiment, use of combinations is provided for treating non-Hodgkin's lymphoma in a human in need thereof wherein the combination comprises:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ ID.NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds OX40 comprising CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, CDRL3 of SEQ. ID. NO: 224, or variants thereof.

Also provided in the present invention is use of a combination or pharmaceutical compositions of this invention in the manufacture of a medicament for the treatment of cancer such as in particular in the treatment of B cell disorders. In one embodiment the cancer is multiple myeloma. In another embodiment the cancer is non-Hodgkin's lymphoma.

In one embodiment, use of a combination or pharmaceutical compositions of this invention in the manufacture of a medicament for the treatment of cancer includes combinations comprising any one of the antigen binding proteins that bind BCMA of the present invention or antibody drug conjugates thereof; and pembrolizumab, or an antibody comprising 90%, 91%, 92%, 93%, 94%, 95%, 96%, 97%, 98%, 99% or 100% sequence homology thereto.

In one embodiment, use of a combination or pharmaceutical compositions of this invention in the manufacture of a medicament for the treatment of cancer includes combinations comprising any one of the antigen binding proteins that bind BCMA or immunoconjugates thereof; and nivolumab, or an antibody comprising 90%, 91%, 92%, 93%, 94%, 95%, 96%, 97%, 98%, 99% or 100% sequence homology thereto

In one embodiment, use of combinations in the manufacture of a medicament for the treatment of cancer is contemplated, wherein the combination comprises:
i) a therapeutically effective amount an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount an antigen binding protein that binds PD-1 comprising CDRH1 of SEQ. ID. NO: 201, CDRH2 of SEQ. ID. NO: 202, CDRH3 of SEQ. ID. NO: 203, CDRL1 of SEQ. ID. NO: 204, CDRL2 of SEQ. ID. NO: 205, CDRL3 of SEQ. ID. NO: 206, or variants thereof.

In one embodiment, use of combinations in the manufacture of a medicament for the treatment of cancer is contemplated, wherein the combination comprises:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds OX40 comprising CDRH1 of SEQ. ID. NO: 219, CDRH2 of SEQ. ID. NO: 220, CDRH3 of SEQ ID NO: 221, CDRL1 of SEQ. ID. NO: 222, CDRL2 of SEQ. ID. NO: 223, CDRL3 of SEQ. ID. NO: 224, or variants thereof.

In one embodiment, use of combinations in the manufacture of a medicament for the treatment of multiple myeloma is contemplated, wherein the combination comprises:
i) a therapeutically effective amount an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ ID. NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount an antigen binding protein that binds PD-1 comprising CDRH1 of SEQ. ID. NO: 201, CDRH2 of SEQ. ID. NO: 202, CDRH3 of SEQ. ID. NO: 203, CDRL1 of SEQ. ID. NO: 204, CDRL2 of SEQ. ID. NO: 205, CDRL3 of SEQ. ID. NO: 206, or variants thereof.

In one embodiment, use of combinations in the manufacture of a medicament for the treatment of non-Hodgkin's lymphoma is contemplated, wherein the combination comprises:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds PD-1 comprising CDRH1 of SEQ. ID. NO: 201, CDRH2 of SEQ. ID. NO: 202, CDRH3 of SEQ. ID. NO: 203, CDRL1 of SEQ. ID. NO: 204, CDRL2 of SEQ. ID. NO: 205, CDRL3 of SEQ. ID. NO: 206, or variants thereof.

In one embodiment, use of combinations in the manufacture of a medicament for the treatment of multiple myeloma is contemplated, wherein the combination comprises:
i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds OX40 comprising CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, CDRL3 of SEQ. ID. NO: 224, or variants thereof.

In one embodiment, use of combinations in the manufacture of a medicament for the treatment of non-Hodgkin's lymphoma is contemplated, wherein the combination comprises: i) a therapeutically effective amount of an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, CDRL3 of SEQ. ID. NO: 6 or variants thereof conjugated to MMAF; and
ii) a therapeutically effective amount of an antigen binding protein that binds OX40 comprising CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, CDRL3 of SEQ. ID. NO: 224, or variants thereof.

Also provided are pharmaceutical compositions comprising the combination of the present invention for treating cancer. The present invention also provides combination kit comprising pharmaceutical compositions of the invention together with one or more pharmaceutically acceptable carriers. The kit may optionally include instructions for use.

B-cell disorders can be divided into defects of B-cell development/immunoglobulin production (immunodeficiencies) and excessive/uncontrolled proliferation (lymphomas, leukemias). As used herein, B-cell disorder refers to both types of diseases, and methods are provided for treating B-cell disorders with an antigen binding protein.

Examples of cancers and in particular B-cell mediated or plasma cell mediated diseases or antibody mediated diseases or disorders include Multiple Myeloma (MM), chronic lymphocytic leukemia (CLL), Follicular Lymphoma (FL), Diffuse Large B-Cell Lymphoma (DLBCL), Non-secretory multiple myeloma, Smoldering multiple myeloma, Monoclonal gammopathy of undetermined significance (MGUS), Solitary plasmacytoma (Bone, Extramedullary), Lymphoplasmacytic lymphoma (LPL), Waldenström's Macroglobulinemia, Plasma cell leukemia, Primary Amyloidosis (AL), Heavy chain disease, Systemic lupus erythematosus (SLE), POEMS syndrome/osteosclerotic myeloma, Type I and II cryoglobulinemia, Light chain deposition disease, Goodpasture's syndrome, Idiopathic thrombocytopenic purpura (ITP), Acute glomerulonephritis, Pemphigus and Pemphigoid disorders, and Epidermolysis bullosa acquisita; or any Non-Hodgkin's Lymphoma B-cell leukemia (NHL) or Hodgkin's lymphoma (HL).

In a particular embodiment, the disease or disorder is selected from the group consisting of Multiple Myeloma (MM), Non-Hodgkin's Lymphoma B-cell leukemia (NHL), Follicular Lymphoma (FL), and Diffuse Large B-Cell Lymphoma (DLBCL).

In one embodiment of the present invention the disease is Multiple Myeloma, or Non-Hodgkin's Lymphoma B-cell leukemia (NHL).

In one embodiment of the present invention the disease is Multiple Myeloma.

Suitably, the present invention relates to a method for treating or lessening the severity of a cancer as herein described.

The combination of the invention may be used alone or in combination with one or more other therapeutic agents.

When a pharmaceutical composition or combination of the present invention is administered for the treatment of cancer, the term “administering” and derivatives thereof as used herein is meant either simultaneous administration or any manner of separate sequential administration of a combination, as described herein, and a further active ingredient or ingredients, known to be useful in the treatment of cancer, including chemotherapy (e.g., and anti-neoplastic agent), radiation treatment, and surgery. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered intravenously and another compound may be administered orally.

Typically, any anti-neoplastic agent that has activity versus a susceptible tumor being treated may be administered with the combinations described herein in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors. A non-limiting list of anti-neoplastic agents are provided herein.

Examples of a further active ingredient or ingredients for administration with the combinations described herein are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)-N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)-N-carboxy-3-phenylisoserine, N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R-(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′, 2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (45)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I—DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I—DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

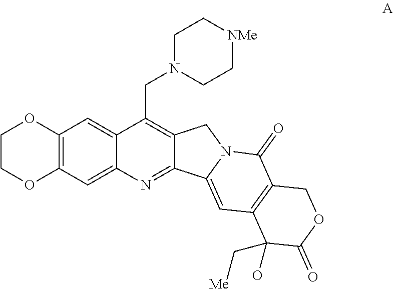 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Letrozole (trade name Femara) is an oral non-steroidal aromatase inhibitor for the treatment of hormonally-responsive breast cancer after surgery. Estrogens are produced by the conversion of androgens through the activity of the aromatase enzyme. Estrogens then bind to an estrogen receptor, which causes cells to divide. Letrozole prevents the aromatase from producing estrogens by competitive, reversible binding to the heme of its cytochrome P450 unit. The action is specific, and letrozole does not reduce production of mineralo- or corticosteroids.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and Bennett, C. F. and Cowsert, L. M. BioChim. Biophys. Acta, (1999) 1489(1):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Pazopanib which commercially available as VOTRIENT® is a tyrosine kinase inhibitor (TKI). Pazopanib is presented as the hydrochloride salt, with the chemical name 5-[[4-[(2,3-dimethyl-2H-indazol-6-yl)methylamino]-2-pyrimidinyl]amino]-2-methylbenzenesulfonamide monohydrochloride. Pazoponib is approved for treatment of patients with advanced renal cell carcinoma.

Bevacisumab which is commercially available as AVASTIN® is a humanized monoclonal antibody that blocks VEGF-A. AVASTIN® is approved form the treatment of various cancers including colorectal, lung, breast, kidney, and glioblastomas.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia CLL; a type of cancer of the white blood cells) in adults who are refractory to treatment with fludarabine (Fludara) and alemtuzumab Campath).

Trastuzumab (HEREPTIN®) is a humanized monoclonal antibody that binds to the HER2 receptor. It original indication is HER2 positive breast cancer. Trastuzumab emtansine (trade name Kadcyla) is anantibody-drug conjugate consisting of the monoclonal antibody trastuzumab (Herceptin) linked to the cytotoxic agent DM1. Trastuzumab alone stops growth of cancer cells by binding to the HER2/neu receptor, whereas DM1 enters cells and destroys them by binding to tubulin. Because the monoclonal antibody targets HER2, and HER2 is only over-expressed in cancer cells, the conjugate delivers the toxin specifically to tumor cells.^{[8]} The conjugate is abbreviated T-DM1.

Cetuximab (ERBITUX®) is a chimeric mouse human antibody that inhibits epidermal growth factor receptor (EGFR).

mTOR inhibitors include but are not limited to rapamycin (FK506) and rapalogs, RAD001 or everolimus (Afinitor), CCI-779 or temsirolimus, AP23573, AZD8055, WYE-354, WYE-600, WYE-687 and Pp121.

Everolimus is sold as Afinitor® by Novartis and is the 40-O-(2-hydroxyethyl) derivative of sirolimus and works similarly to sirolimus as an mTOR (mammalian target of rapamycin) inhibitor. It is currently used as an immunosuppressant to prevent rejection of organ transplants and treatment of renal cell cancer. Much research has also been conducted on everolimus and other mTOR inhibitors for use in a number of cancers. It has the following chemical structure (formula II) and chemical name:

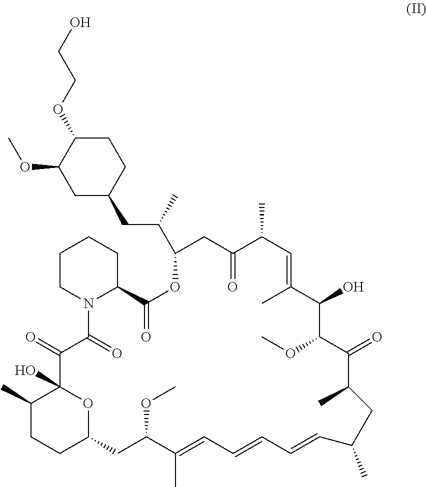 [SMILES: CO[C@H]1C[C@@H]2CC[C@@H](C)[C@@](O)(O2)C(=O)C(=O)N2CCCC[C@H]2C(=O)O[C@H]([C@@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone.

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl) ethenyl] benzoic acid. Bexarotene is used to treat cutaneous T-cell lymphoma (CTCL, a type of skin cancer) in people whose disease could not be treated successfully with at least one other medication.

Sorafenib marketed as Nexavar® is in a class of medications called multikinase inhibitors. Its chemical name is 4-[4-[[4-chloro-3-(trifluoromethyl)phenyl]carbamoylamino] phenoxy]-N-methyl-pyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Examples of erbB inhibitors include lapatinib, erlotinib, and gefitinib. Lapatinib, N-(3-chloro-4-{[(3-fluorophenyl)methyl]oxy}phenyl)-6-[5-({[2-(methylsulfonyl)ethyl]amino}methyl)-2-furanyl]-4-quinazolinamine (represented by Formula III, as illustrated), is a potent, oral, small-molecule, dual inhibitor of erbB-1 and erbB-2 (EGFR and HER2) tyrosine kinases that is approved in combination with capecitabine for the treatment of HER2-positive metastatic breast cancer.

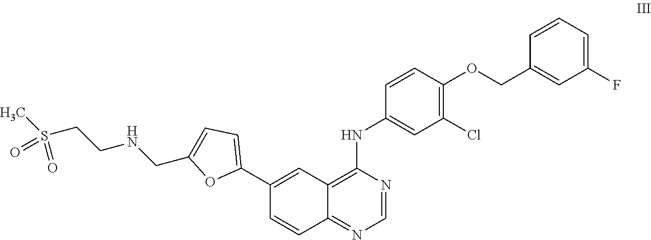 [SMILES: CS(=O)(=O)CCNCc1ccc(-c2ccc3ncnc(Nc4ccc(OCc5cccc(F)c5)c(Cl)c4)c3c2)o1]

The free base, HCl salts, and ditosylate salts of the compound of formula (III) may be prepared according to the procedures disclosed in WO 99/35146, published Jul. 15, 1999; and WO 02/02552 published Jan. 10, 2002.

Erlotinib, N-(3-ethynylphenyl)-6,7-bis{[2-(methyloxy)ethyl]oxy}-4-quinazolinamine Commercially available under the tradename Tarceva) is represented by formula IV, as illustrated:

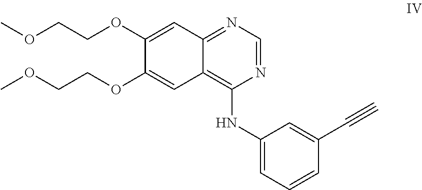 [SMILES: C#Cc1cccc(Nc2ncnc3cc(OCCOC)c(OCCOC)cc23)c1]

The free base and HCl salt of erlotinib may be prepared, for example, according to U.S. Pat. No. 5,747,498, Example 20.

Gefitinib, 4-quinazolinamine, N-(3-chloro-4-fluorophenyl)-7-methoxy-6-[3-4-morpholin)propoxy] is represented by formula V, as illustrated:

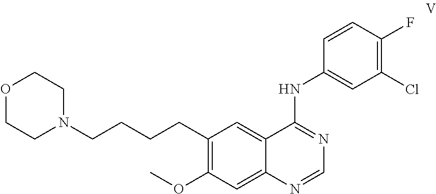 [SMILES: COc1cc2ncnc(Nc3ccc(F)c(Cl)c3)c2cc1CCCCN1CCOCC1]

Gefitinib, which is commercially available under the trade name IRESSA® (Astra-Zenenca) is an erbB-1 inhibitor that is indicated as monotherapy for the treatment of patients with locally advanced or metastatic non-small-cell lung cancer after failure of both platinum-based and docetaxel chemotherapies. The free base, HCl salts, and diHCl salts of gefitinib may be prepared according to the procedures of International Patent Application No. PCT/GB96/00961, filed Apr. 23, 1996, and published as WO 96/33980 on Oct. 31, 1996.

Trastuzumab (HEREPTIN®) is a humanized monoclonal antibody that binds to the HER2 receptor. It original indication is HER2 positive breast cancer.

Cetuximab (ERBITUX®) is a chimeric mouse human antibody that inhibits epidermal growth factor receptor (EGFR).

Pertuzumab (also called 2C4, trade name Omnitarg) is a monoclonal antibody. The first of its class in a line of agents called “HER dimerization inhibitors”. By binding to HER2, it inhibits the dimerization of HER2 with other HER receptors, which is hypothesized to result in slowed tumor growth. Pertuzumab is described in WO01/00245 published Jan. 4, 2001.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

As used herein “immuno-modulators” refer to any substance including monoclonal antibodies that effects the immune system. The PD-1 and OX40 antigen binding proteins of the present invention can be considered immune-modulators. Immuno-modulators can be used as anti-neoplastic agents for the treatment of cancer. For example, immune-modulators include, but are not limited to, anti-CTLA-4 antibodies such as ipilimumab (YERVOY®) and anti-PD-1 antibodies (Opdivo/nivolumab and Keytruda/pembrolizumab). Other immuno-modulators include, but are not limited to, OX-40 antibodies, PD-L1 antibodies, LAG3 antibodies, TIM-3 antibodies, 41BB antibodies and GITR antibodies.

YERVOY® (ipilimumab) is a fully human CTLA-4 antibody marketed by Bristol Myers Squibb. The protein structure of ipilimumab and methods are using are described in U.S. Pat. Nos. 6,984,720 and 7,605,238.

OPDIVO®/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

KEYTRUDA®/pembrolizumab is an anti-PD-1 antibodies marketed for the treatment of lung cancer by Merck. The amino acid sequence of pembrolizumab and methods of using are disclosed in U.S. Pat. No. 8,168,757.

CD134, also known as OX40, is a member of the TNFR-superfamily of receptors which is not constitutively expressed on resting naïve T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. OX-40 antibodies, OX-40 fusion proteins and methods of using them are disclosed in U.S. Pat. Nos. 7,504,101; 7,758,852; 7,858,765; 7,550,140; 7,960,515; WO2012027328; WO2013028231.

Antibodies to PD-L1 (also referred to as CD274 or B7-H1) and methods for use are disclosed in U.S. Pat. Nos. 7,943,743; 8,383,796; US20130034559, WO2014055897, U.S. Pat. No. 8,168,179; and 7,595,048. PD-L1 antibodies are in development as immuno-modulatory agents for the treatment of cancer.

In another embodiment, methods are provided of treating cancer in a mammal in need thereof comprising: administering to such mammal a therapeutically effective amount of
a) a combination of the present invention; and
b) at least one anti-neoplastic agent.

In another embodiment, methods are provided of treating cancer in a mammal in need thereof comprising: administering to such mammal a therapeutically effective amount of
a) a combination of the present invention; and
b) at least one immune-modulator.

In one embodiment methods are provided for treating cancer in a human in need thereof comprising administering a therapeutically effective amount of a combination of the present invention wherein the combination comprises a therapeutically effective amount of an antigen binding protein that binds BCMA and a therapeutically effective amount of an antigen binding protein that binds PD-1 and a therapeutically effective amount of an antigen binding protein that binds OX-40.

In the embodiment, the combination of the invention may be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged.

In one embodiment, the further anti-cancer therapy is surgical and/or radiotherapy. In one embodiment, the further anti-cancer therapy is at least one additional anti-neoplastic agent.

Any anti-neoplastic agent that has activity versus a susceptible tumor being treated may be utilized in the combination. Typical anti-neoplastic agents useful include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine angiogenesis inhibitors; immunotherapeutic agents; proapoptotic agents; and cell cycle signaling inhibitors.

In one embodiment, the combination of the present invention comprises an anti-BCMA antigen binding protein and either a PD-1 or OX40 antigen binding protein and at least one anti-neoplastic agent selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine MEKngiogenesis inhibitors, immunotherapeutic agents, proapoptotic agents, and cell cycle signaling inhibitors.

In one embodiment, the combination of the present invention comprises an anti-BCMA antigen binding protein and a PD-1 or OX40 antigen binding protein and at least one anti-neoplastic agent which is an anti-microtubule agent selected from diterpenoids and vinca alkaloids.

In a further embodiment, the at least one anti-neoplastic agent is a diterpenoid.

In a further embodiment, the at least one anti-neoplastic agent is a vinca alkaloid.

In one embodiment, the combination of the present invention comprises an anti-BCMA antigen binding protein and a PD-1 or OX40 antigen binding protein and at least one anti-neoplastic agent, which is a platinum coordination complex.

In a further embodiment, the at least one anti-neoplastic agent is paclitaxel, carboplatin, or vinorelbine.

In a further embodiment, the at least one anti-neoplastic agent is carboplatin.

In a further embodiment, the at least one anti-neoplastic agent is vinorelbine.

In a further embodiment, the at least one anti-neoplastic agent is paclitaxel.

In one embodiment, the combination of the present invention comprises an anti-BCMA antigen binding protein and a PD-1 or OX40 antigen binding protein and at least one anti-neoplastic agent which is a signal transduction pathway inhibitor.

In a further embodiment the signal transduction pathway inhibitor is an inhibitor of a growth factor receptor kinase VEGFR2, TIE2, PDGFR, BTK, erbB2, EGFr, IGFR-1, TrkA, TrkB, TrkC, or c-fms.

In a further embodiment the signal transduction pathway inhibitor is an inhibitor of a serine/threonine kinase rafk, akt, or PKC-zeta.

In a further embodiment the signal transduction pathway inhibitor is an inhibitor of a non-receptor tyrosine kinase selected from the src family of kinases.

In a further embodiment the signal transduction pathway inhibitor is an inhibitor of c-src.

In a further embodiment the signal transduction pathway inhibitor is an inhibitor of Ras oncogene selected from inhibitors of farnesyl transferase and geranylgeranyl transferase.

In a further embodiment the signal transduction pathway inhibitor is an inhibitor of a serine/threonine kinase selected from the group consisting of PI3K.

In a further embodiment the signal transduction pathway inhibitor is a dual EGFr/erbB2 inhibitor, for example N-{3-Chloro-4-[(3-fluorobenzyl) oxy]phenyl}-6-[5-({[2-(methanesulphonyl) ethyl]amino}methyl)-2-furyl]-4-quinazolinamine (structure below):

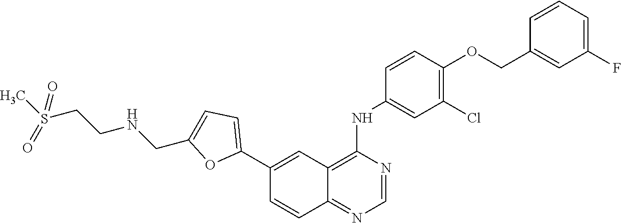 [SMILES: CS(=O)(=O)CCNCc1ccc(-c2ccc3ncnc(Nc4ccc(OCc5cccc(F)c5)c(Cl)c4)c3c2)o1]

### Definitions

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor (3) mimics one or more functions of a natural ligand or molecule that interacts with a target or receptor and includes initiating one or more signaling events through the receptor, mimicking one or more functions of a natural ligand, or initiating one or more partial or full conformational changes that are seen in known functioning or signaling through the receptor and/or (4) enhances, increases, promotes or induces the expression of the receptor. Agonist activity can be measured in vitro by various assays known in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a co-signalling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, decrease, abrogates the expression of the receptor. Antagonist activity can be measured in vitro by various assays known in the art such as, but not limited to, measurement of an increase or decrease in cell signalling, cell proliferation, immune cell activation markers, cytokine production. Antagonist activity can also be measured in vivo by various assays that measure surrogate end points such as, but not limited to the measurement of T cell proliferation or cytokine production.

Thus, as used herein the term “combination of the invention” refers to a combination comprising an anti-BCMA antigen binding protein suitably an antagonist anti-BCMA antigen binding protein and either a PD-1 antigen binding protein, suitably an antagonist anti-PD-1 antigen binding protein or an OX40 antigen binding protein, suitably an agonistic OX40 antigen binding protein each of which may be administered separately or simultaneously as described herein.

As used herein, the terms “cancer,” “neoplasm,” and “tumor,” are used interchangeably and in either the singular or plural form, refer to cells that have undergone a malignant transformation or undergone cellular changes that result in aberrant or unregulated growth or hyperproliferation Such changes or malignant transformations usually make such cells pathological to the host organism, thus precancers or precancerous cells that are or could become pathological and require or could benefit from intervention are also intended to be included. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. In other words, the terms herein include cells, neoplasms, cancers, and tumors of any stage, including what a clinician refers to as precancer, tumors, in situ growths, as well as late stage metastatic growths, Tumors may be hematopoietic tumor, for example, tumors of blood cells or the like, meaning liquid tumors. Specific examples of clinical conditions based on such a tumor include leukemia such as chronic myelocytic leukemia or acute myelocytic leukemia; myeloma such as multiple myeloma; lymphoma and the like.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

By the term “treating” and derivatives thereof as used herein, is meant therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Antigen Binding Protein” means a protein that binds an antigen, including antibodies or engineered molecules that function in similar ways to antibodies. Such alternative antibody formats include triabody, tetrabody, miniantibody, and a minibody, Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP of a combination of the invention, or a method or use thereof, may comprise the VH regions formatted into a full length antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The antigen binding protein may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533 which comprises an antigen binding region and a non-immunoglobulin region.

An antigen binding protein may also be a chimeric antigen receptor. The term “chimeric antigen receptors” (“CARs”) as used herein, refers to an engineered receptor which consists of an extracellular target binding domain (which is usually derived from a monoclonal antibody), a spacer region, a transmembrane region, and one or more intracellular effector domains. CARs have also been referred to as chimeric T cell receptors or chimeric immunoreceptors (CIRs). CARs are genetically introduced into hematopoietic cells, such as T cells, to redirect specificity for a desired cell-surface antigen.

Chimeric antigen receptors (CARs) have been developed as artificial TCRs to generate novel specificities in T cells without the need to bind to MHC-antigenic peptide complexes. These synthetic receptors contain a target binding domain that is associated with one or more signalling domains via a flexible linker in a single fusion molecule. The target binding domain is used to target the T cell to specific targets on the surface of pathologic cells and the signalling domains contain molecular machinery for T cell activation and proliferation. The flexible linker which passes through the T cell membrane (i.e. forming a transmembrane domain) allows for cell membrane display of the target binding domain of the CAR. CARs have successfully allowed T cells to be redirected against antigens expressed at the surface of tumour cells from various malignancies including lymphomas and solid tumours (Jena et al. (2010) Blood, 116(7):1035-44).

The development of CARs has comprised three generations so far. The first generation CARs comprised target binding domains attached to a signalling domain derived from the cytoplasmic region of the CD3zeta or the Fc receptor gamma chains. First generation CARs were shown to successfully redirect T cells to the selected target, however, they failed to provide prolonged expansion and antitumor activity in vivo. The second and third generation CARs have focussed on enhancing modified T cell survival and increasing proliferation by including costimulatory molecules, such as CD28, OX-40 (CD134) and 4-1BB (CD137).

T cells bearing CARs could be used to eliminate pathologic cells in a disease setting. One clinical aim would be to transform patient cells with recombinant DNA containing an expression construct for the CAR via a vector (e.g. a lentiviral vector) following aphaeresis and T cell isolation. Following expansion of the T cells they are re-introduced into the patient with the aim of targeting and killing the pathologic target cells.

The term “antibody” as used herein refers to molecules with an antigen binding domain, and optionally an immunoglobulin-like domain or fragment thereof and includes monoclonal (for example IgG, IgM, IgA, IgD or IgE and modified variants thereof), recombinant, polyclonal, chimeric, humanized, biparatopic, bispecific and heteroconjugate antibodies, or a closed conformation multispecific antibody. An “antibody” included xenogeneic, allogeneic, syngeneic, or other modified forms thereof. An antibody may be isolated or purified. An antibody may also be recombinant, i.e. produced by recombinant means; for example, an antibody that is 90% identical to a reference antibody may be generated by mutagenesis of certain residues using recombinant molecular biology techniques known in the art. Thus, the antibodies of the present invention may comprise heavy chain variable regions and light chain variable regions of a combination of the invention, or a method or use thereof, which may be formatted into the structure of a natural antibody or formatted into a full length recombinant antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The antibody may be an IgG1, IgG2, IgG3, or IgG4 or a modified variant thereof. The constant domain of the antibody heavy chain may be selected accordingly. The light chain constant domain may be a kappa or lambda constant domain. The antibody may also be a chimeric antibody of the type described in WO86/01533 which comprises an antigen binding region and a non-immunoglobulin region.

One of skill in the art will recognize that the antigen binding proteins of the invention bind to an epitope on their targets. The epitope of an antigen binding protein is the region of its antigen to which the antigen binding protein binds. Two antigen binding proteins bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference). Alternatively, two antibodies have the same epitope if essentially all amino acid mutations in the antigen that reduce or eliminate binding of one antibody reduce or eliminate binding of the other. Also the same epitope may include “overlapping epitopes” e.g. if some amino acid mutations that reduce or eliminate binding of one antibody reduce or eliminate binding of the other.

The strength of binding may be important in dosing and administration of an antigen binding protein of the combination, or method or use thereof, of the invention. In one embodiment, the antigen binding protein of the invention binds its target (e.g. BCMA or PD-1 or OX40) with high affinity. Affinity is the strength of binding of one molecule, e.g. an antibody of a combination of the invention, or a method or use thereof, to another, e.g. its target antigen, at a single binding site. The binding affinity of an antibody to its target may be determined by equilibrium methods (e.g. enzyme-linked immunoabsorbent assay (ELISA) or radioimmunoassay (RIA)), or kinetics (e.g. BIACORE analysis). For example, the Biacore methods known in the art may be used to measure binding affinity.

Avidity is the sum total of the strength of binding of two molecules to one another at multiple sites, e.g. taking into account the valency of the interaction. Functional fragments of the antigen binding proteins of a combination of the invention, or a method or use thereof, are contemplated herein.

Thus, “binding fragments” and “functional fragments” may be an Fab and F(ab′)2 fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. (1973) Biochemistry 12:1130-1135; Sharon, J. et al. (1976) Biochemistry 15:1591-1594). These various fragments are produced using conventional techniques such as protease cleavage or chemical cleavage (see, e.g., Rousseaux et al., Meth. Enzymol., 121:663-69 (1986)).

“Functional fragments” as used herein means a portion or fragment of the antigen binding proteins of a combination of the invention, or a method or use thereof, that include the antigen-binding site and are capable of binding the same target as the parent antigen binding protein, e.g. but not limited to binding the same epitope, and that also retain one or more modulating or other functions described herein or known in the art.

As the antigen binding proteins of the present invention may comprise heavy chain variable regions and light chain variable regions of a combination of the invention, or a method or use thereof, which may be formatted into the structure of a natural antibody, a functional fragment is one that retains binding or one or more functions of the full length antigen binding protein as described herein. A binding fragment of an antigen binding protein of a combination of the invention, or a method or use thereof, may therefore comprise the VL or VH regions, a (Fab′)2 fragment, a Fab fragment, a fragment of a bi-specific or biparatopic molecule or equivalent thereof (such as scFV, bi- tri- or tetra-bodies, Tandabs etc.), when paired with an appropriate light chain.

The term “CDR” as used herein, refers to the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin.

It will be apparent to those skilled in the art that there are various numbering conventions for CDR sequences; Chothia (Chothia et al. (1989) Nature 342: 877-883), Kabat (Kabat et al., Sequences of Proteins of Immunological Interest, 4th Ed., U.S. Department of Health and Human Services, National Institutes of Health (1987)), AbM (University of Bath) and Contact (University College London). The minimum overlapping region using at least two of the Kabat, Chothia, AbM and contact methods can be determined to provide the “minimum binding unit”. The minimum binding unit may be a subportion of a CDR. The structure and protein folding of the antibody may mean that other residues are considered part of the CDR sequence and would be understood to be so by a skilled person. It is noted that some of the CDR definitions may vary depending on the individual publication used.

Unless otherwise stated and/or in absence of a specifically identified sequence, references herein to “CDR”, “CDRL1” (or “LC CDR1”), “CDRL2” (or “LC CDR2”), “CDRL3” (or “LC CDR3”), “CDRH1” (or “HC CDR1”), “CDRH2” (or “HC CDR2”), “CDRH3” (or “HC CDR3”) refer to amino acid sequences numbered according to any of the known conventions; alternatively, the CDRs are referred to as “CDR1,” “CDR2,” “CDR3” of the variable light chain and “CDR1,” “CDR2,” and “CDR3” of the variable heavy chain. In particular embodiments, the numbering convention is the Kabat convention.

The term “variant” as used herein refers to a heavy chain variable region or light chain variable region that has been modified by at least one, for example 1, 2 or 3, amino acid substitution(s), deletion(s) or addition(s), wherein the modified antigen binding protein comprising the heavy chain or light chain variant substantially retains the biological characteristics of the antigen binding protein pre-modification. In one embodiment, the antigen binding protein containing a variant heavy chain variable region or light chain variable region sequence retains 60%, 70%, 80%, 90%, 100% biological characteristics of the antigen binding protein pre-modification. It will be appreciated that each heavy chain variable region or light chain variable region may be modified alone or in combination with another heavy chain variable region or light chain variable region. The antigen binding proteins of the disclosure include heavy chain variable region amino acid sequences that are 90%, 91%, 92%, 93%, 94%, 95%, 96%, 97%, 98%, or 99% homologous to the heavy chain variable region amino acid sequences described herein. The antigen binding proteins of the disclosure include light chain variable region amino acid sequence that are 90%, 91%, 92%, 93%, 94%, 95%, 96%, 97%, 98%, or 99% homologous to the light chain variable region amino acid sequences described herein.

The percent homology can be over the entire heavy chain variable region and/or entire light chain variable region or the percent homology can be confined to the framework regions, while the sequences that correspond to CDRs have 100% identity to the CDRs disclosed herein within the heavy chain variable region and/or light chain variable region.

The term “CDR variant” as used herein, refers to a CDR that has been modified by at least one, for example 1, 2 or 3, amino acid substitution(s), deletion(s) or addition(s), wherein the modified antigen binding protein comprising the CDR variant substantially retains the biological characteristics of the antigen binding protein pre-modification. In one embodiment, the antigen binding protein containing a variant CDR retains 60%, 70%, 80%, 90%, 100% biological characteristics of the antigen binding protein pre-modification. It will be appreciated that each CDR that can be modified may be modified alone or in combination with another CDR. In one embodiment, the modification is a substitution, particularly a conservative substitution, for example as shown in Table 1.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in one CDR variant, the amino acid residues of the minimum binding unit may remain the same, but the flanking residues that comprise the CDR as part of the Kabat or Chothia definition(s) may be substituted with a conservative amino acid residue.

Such antigen binding proteins comprising modified CDRs or minimum binding units as described above may be referred to herein as “functional CDR variants” or “functional binding unit variants”.

The antibody may be of any species, or modified to be suitable to administer to a cross species. For example the CDRs from a mouse antibody may be humanized for administration to humans. In any embodiment, the antigen binding protein is optionally a humanized antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl Acad Sci USA, 86:10029-10032 (1989), Hodgson et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951.

In yet a further embodiment, the humanized antibody has a human antibody constant region that is an IgG. In another embodiment, the IgG is a sequence as disclosed in any of the above references or patent publications.

“Enhanced FcγRIIIA mediated effector function” as used herein denotes that the usual effector function of the antigen binding protein has been deliberately increased compared to its usual levels. This may be carried out by any means known in the art for example by mutations which increase affinity for FcYRIIIA binding or by alteration of the glycosylation of the antigen binding protein (for example knock out a fucosyltransferase)

For nucleotide and amino acid sequences, the term “identical” or “identity” indicates the degree of identity between two nucleic acid or two amino acid sequences when optimally aligned and compared with appropriate insertions or deletions.

The percent sequence identity between two sequences is a function of the number of identical positions shared by the sequences (i.e., % identity=number of identical positions/total number of positions multiplied by 100), taking into account the number of gaps, and the length of each gap, which need to be introduced for optimal alignment of the two sequences. The comparison of sequences and determination of percent identity between two sequences can be accomplished using a mathematical algorithm, as described below.

Percent identity between a query nucleic acid sequence and a subject nucleic acid sequence is the “Identities” value, expressed as a percentage, which is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

Percent identity between a query amino acid sequence and a subject amino acid sequence is the “Identities” value, expressed as a percentage, which is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

In one embodiment of the invention as herein described the antigen binding proteins have an amino acid sequence with at least 90%, 91%, 92%, 93%, 94%, 95%, 96%, 97%, 98%, 99% or 100% sequence identity to the amino acid sequences as set forth in the sequence listing. In a particular embodiment the antigen binding proteins have at least 98% for example 99% sequence identity to those found in the sequence listing.

Any references or publications described herein are hereby incorporated by reference.

### Examples

The following examples illustrate various non-limiting aspects of this invention.

### Example 1. Production of BCMA Antigen Binding Proteins

Production of antigen binding proteins according to the invention as herein described and conjugation to a toxin and the respective binding affinities of such antigen binding proteins can be found in WO2012163805 as herein incorporated by reference.

### Example 2—Immunogenic Cell Death (ICD)

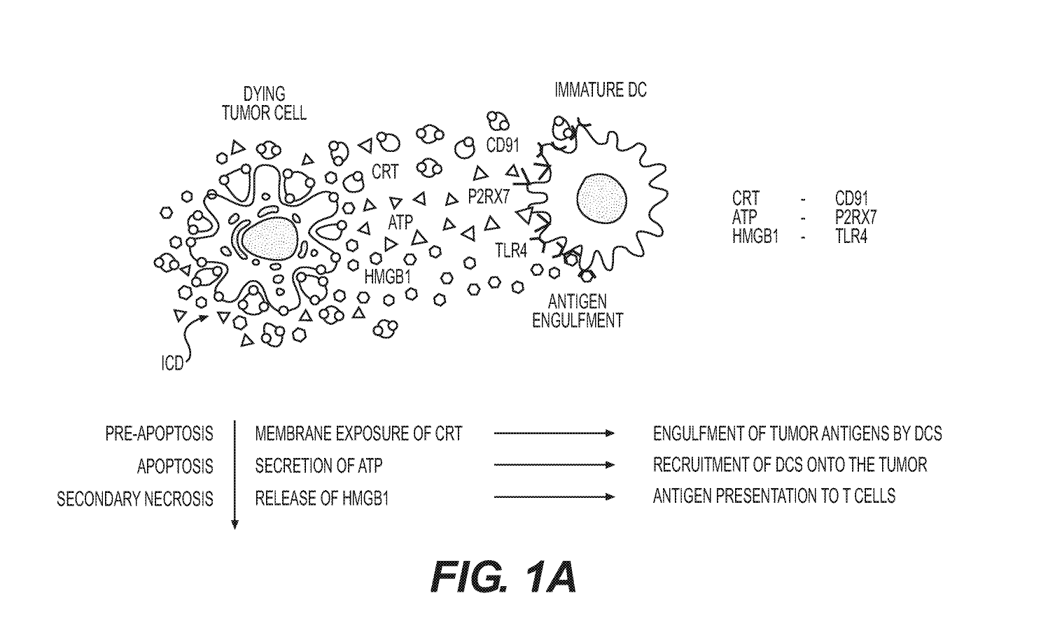

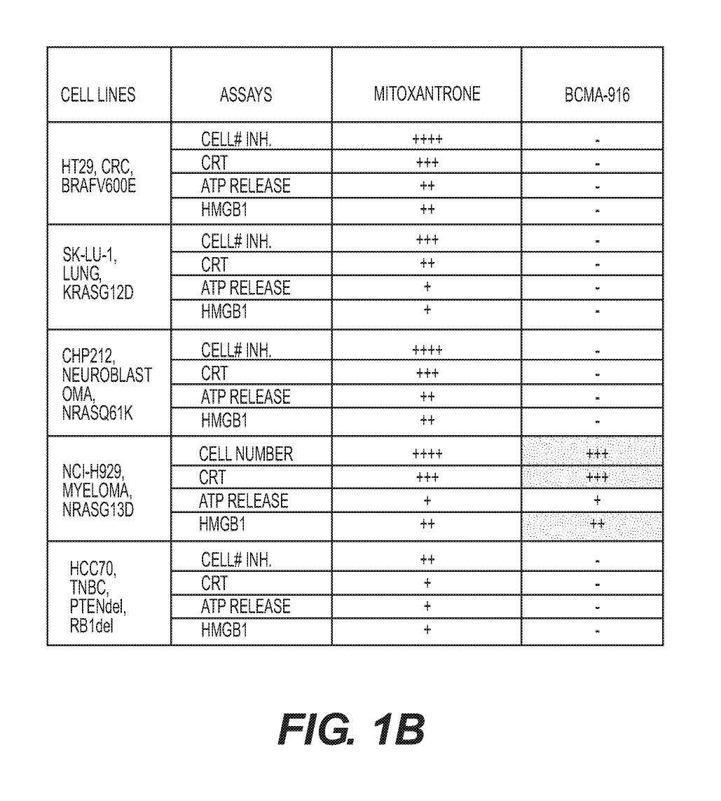

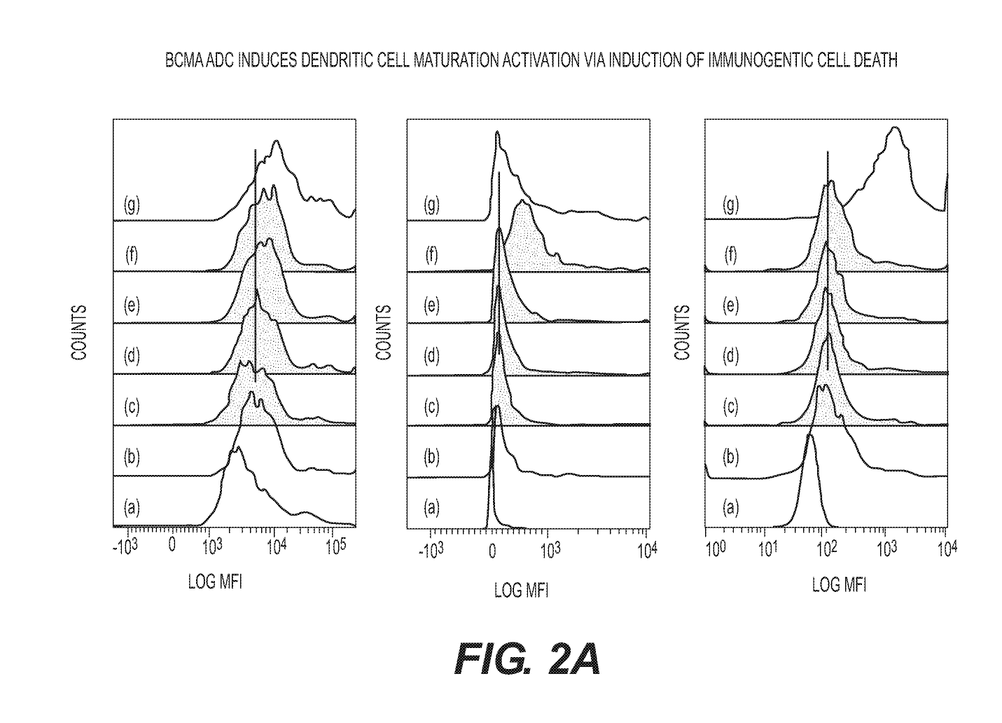

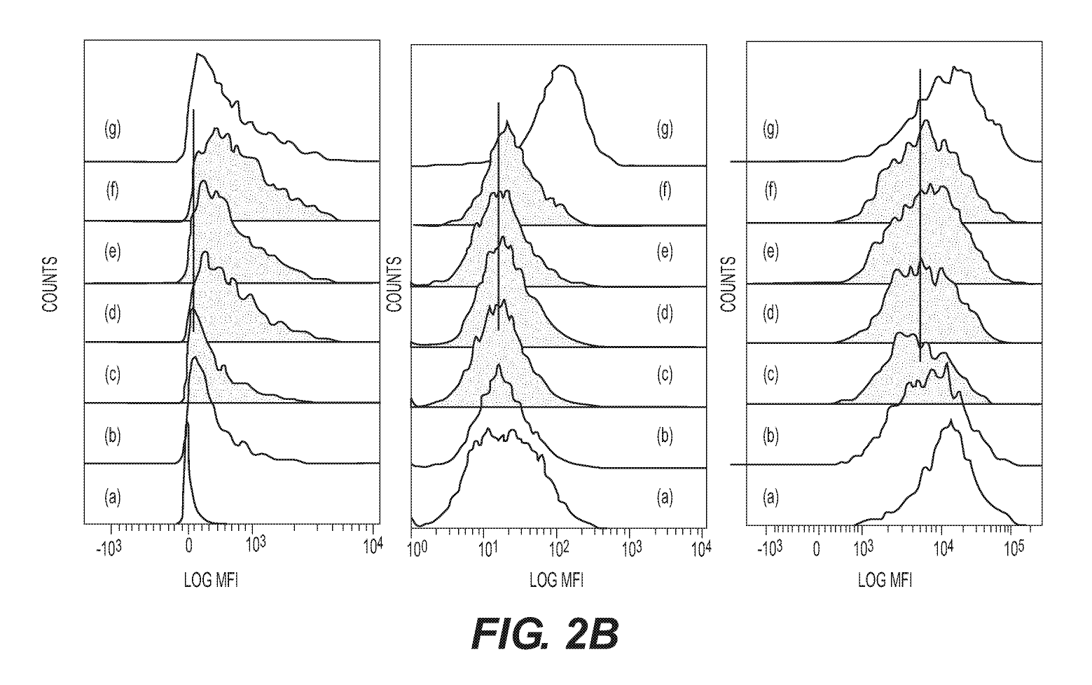

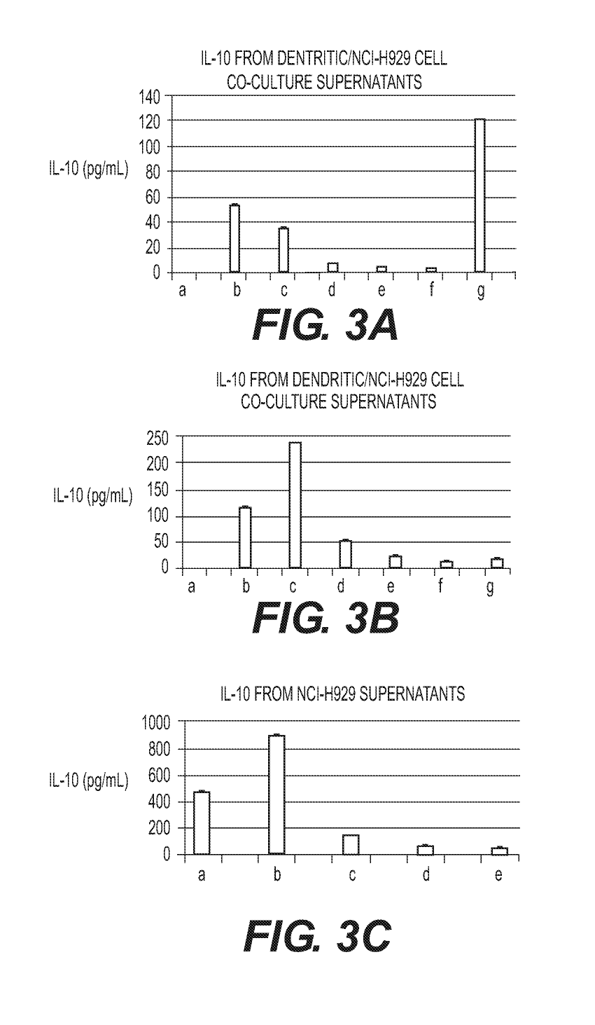

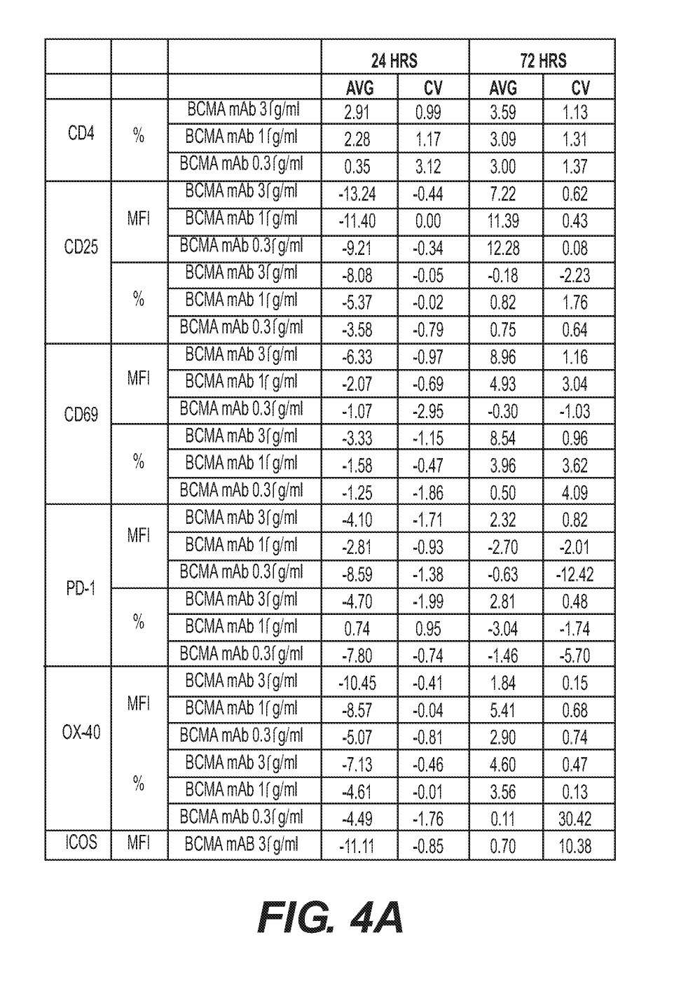

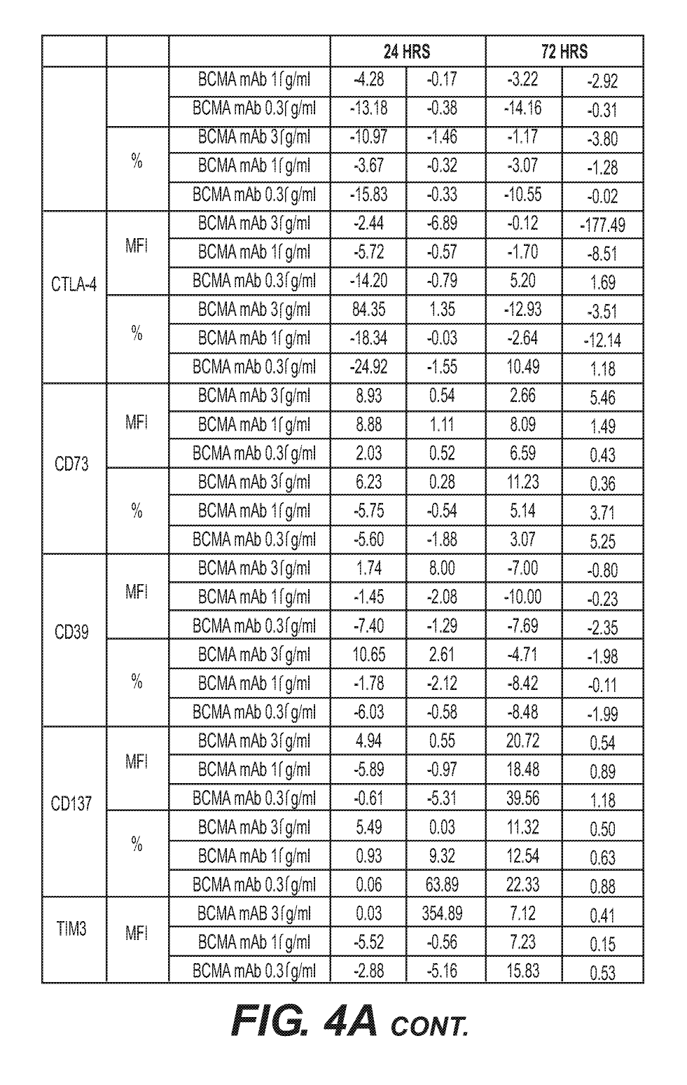

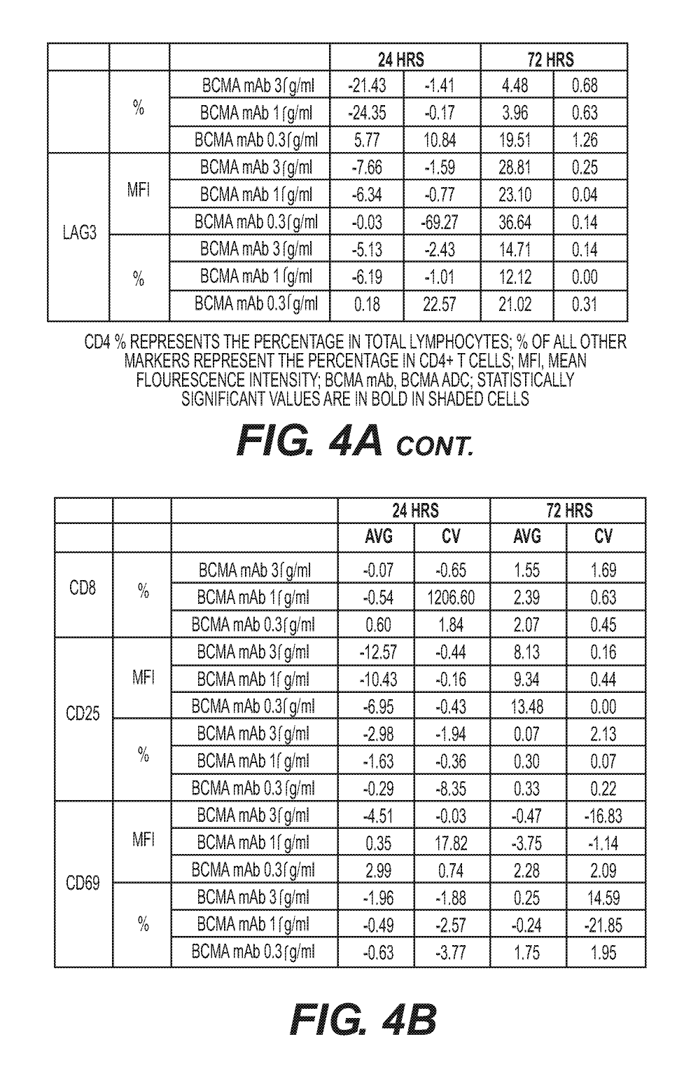

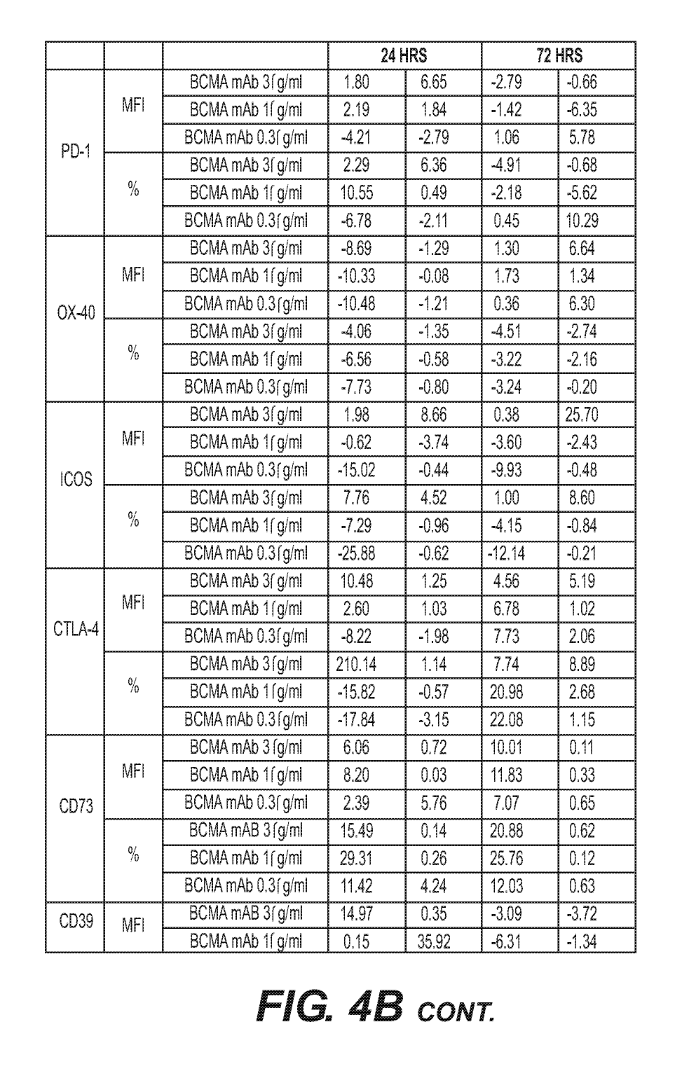

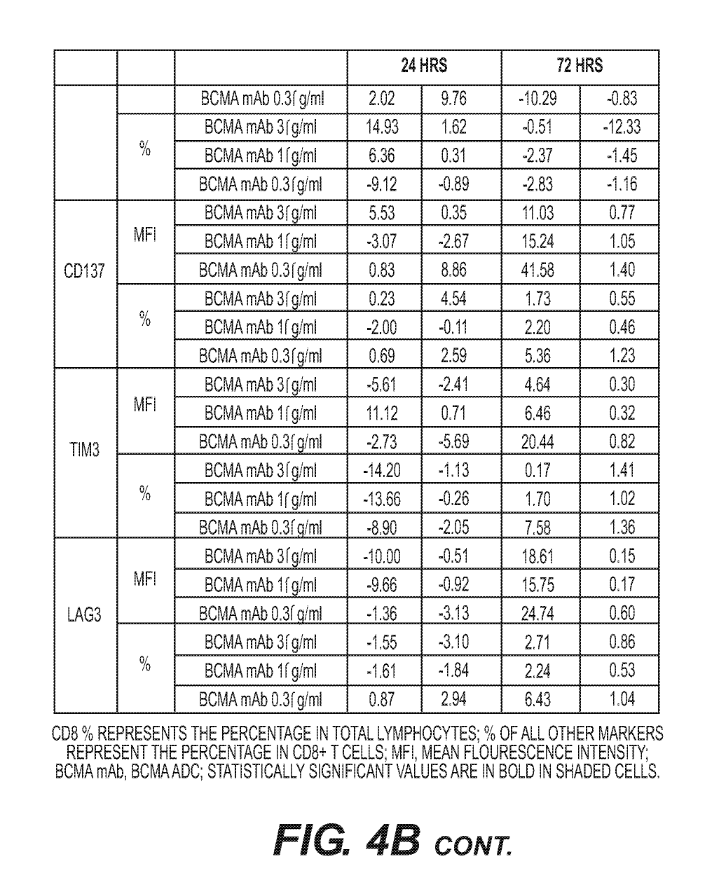

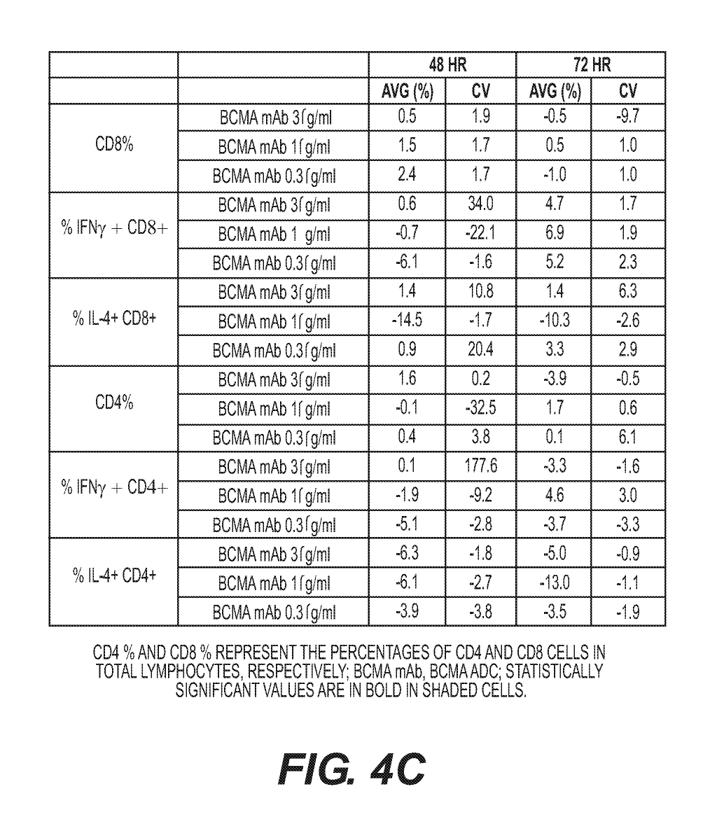

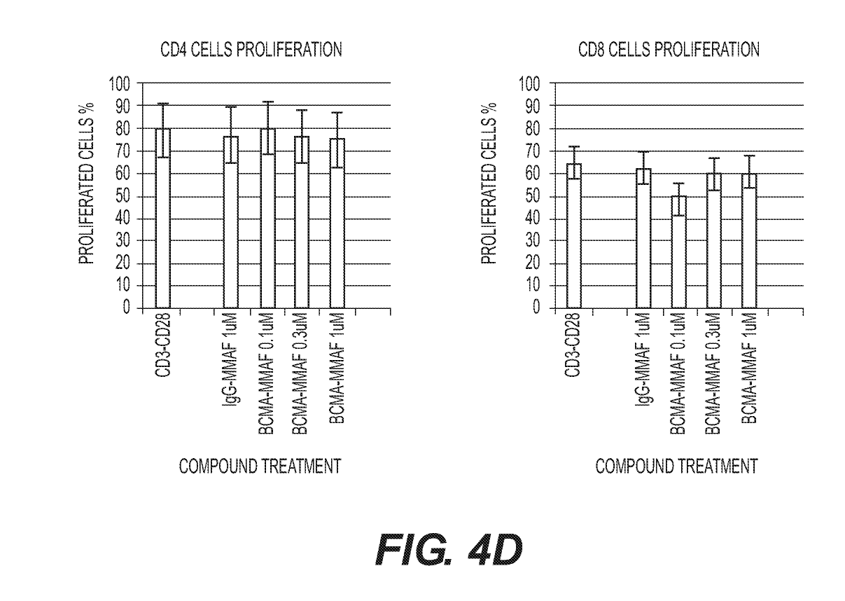

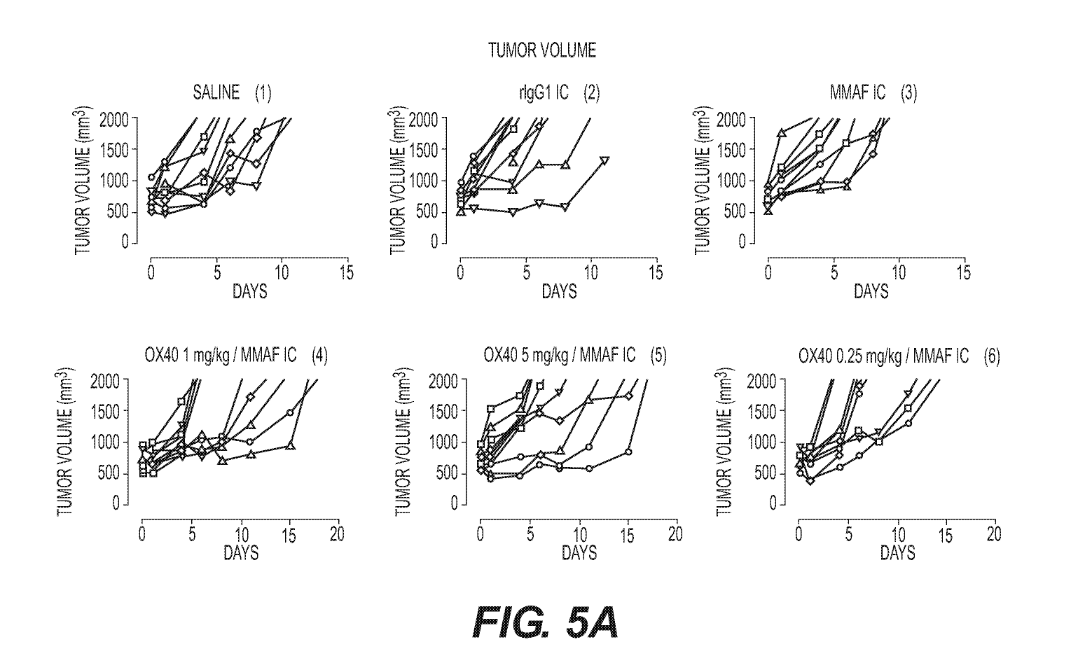

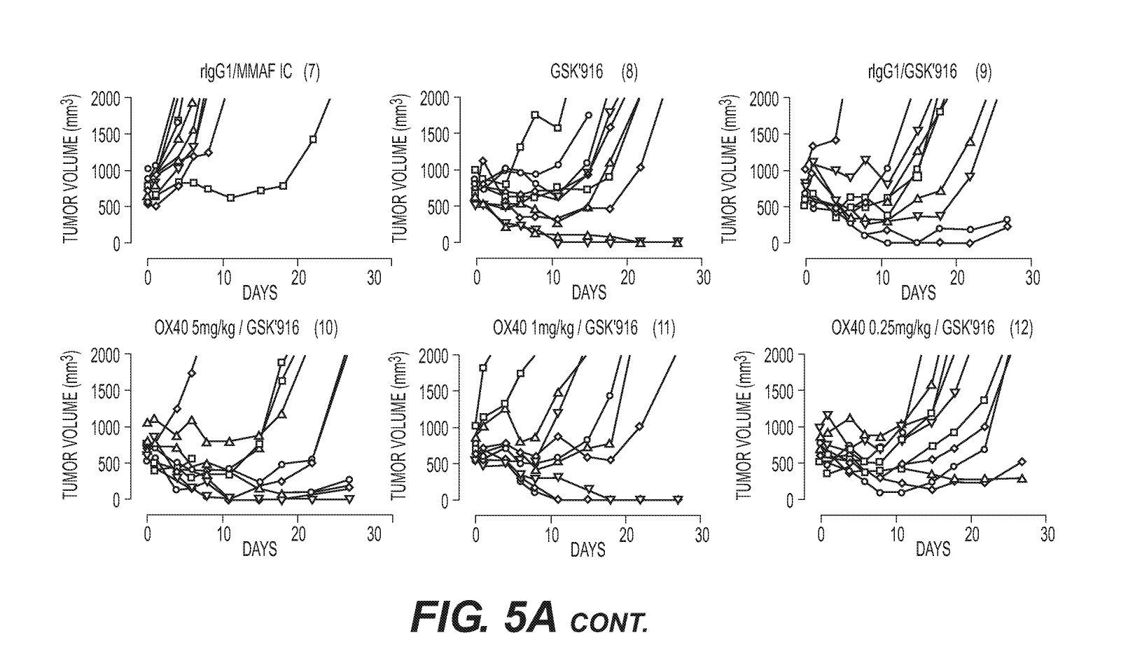

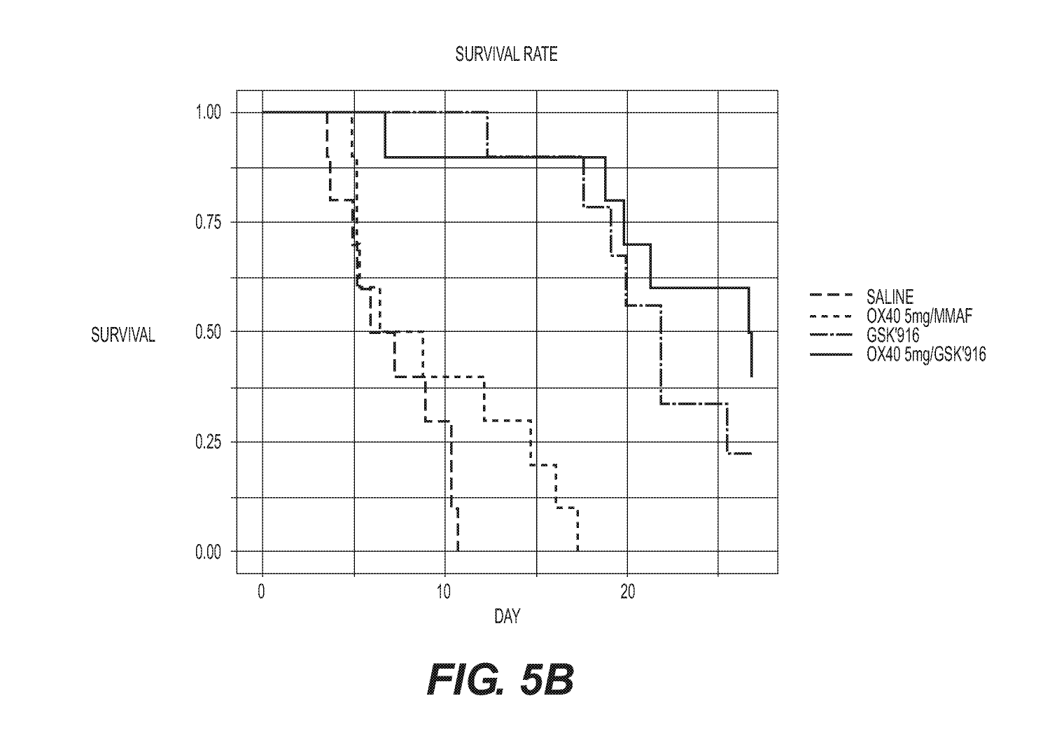

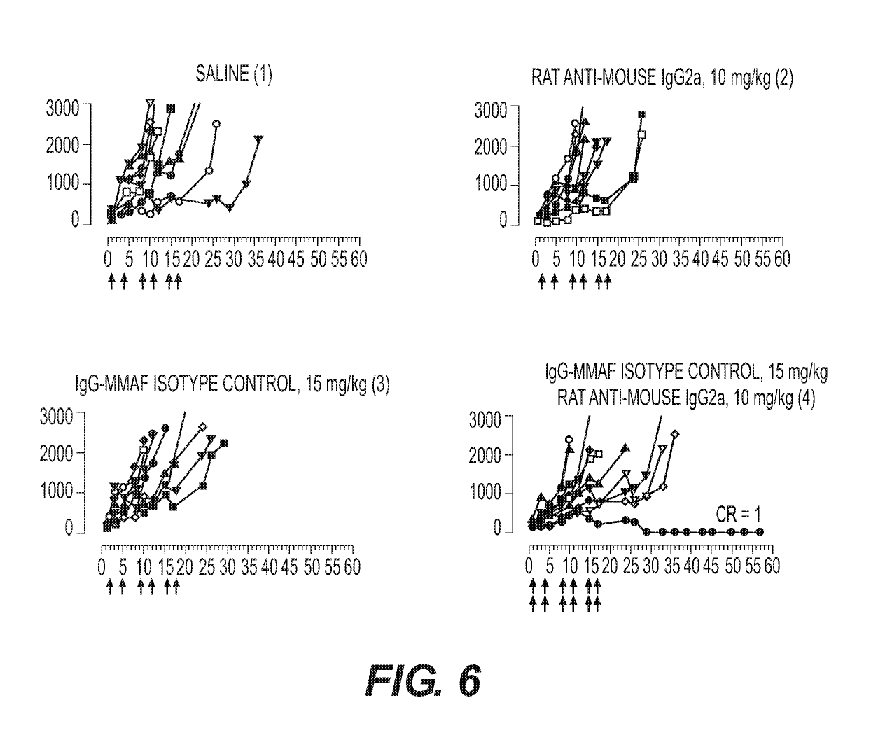

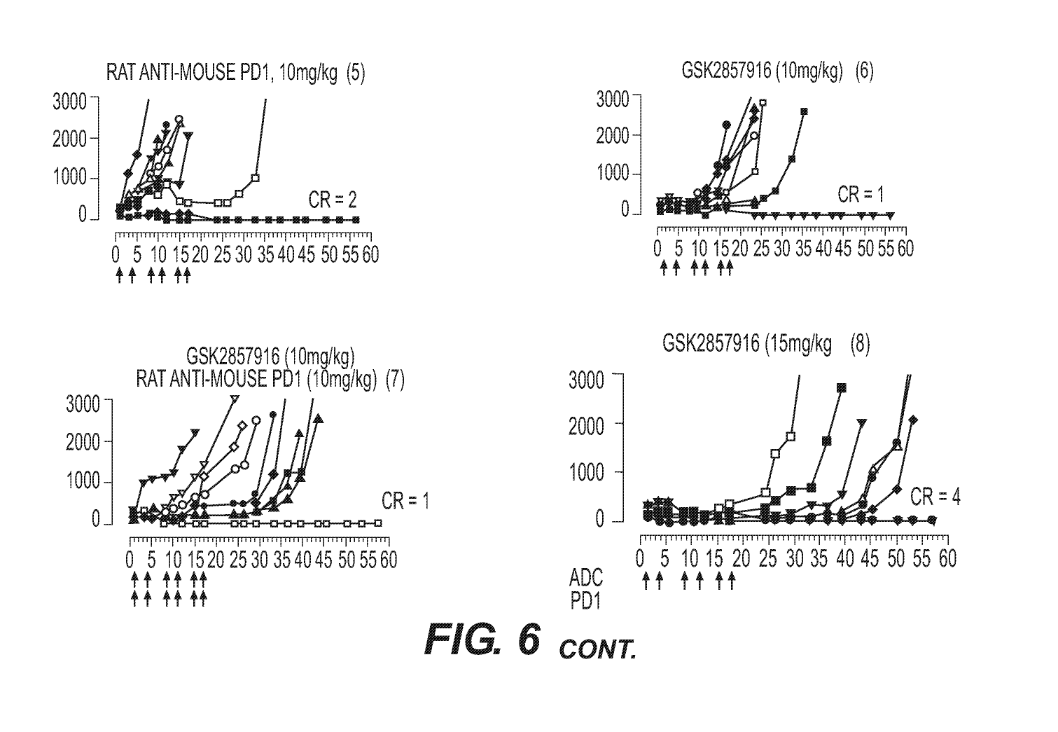

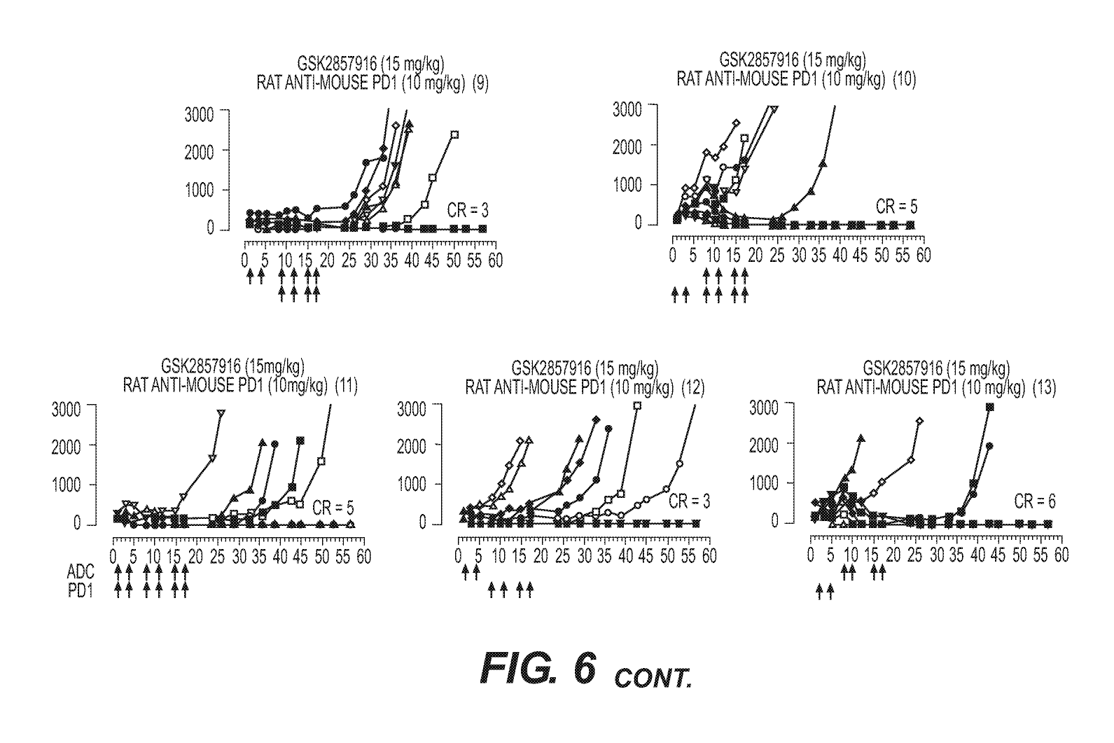

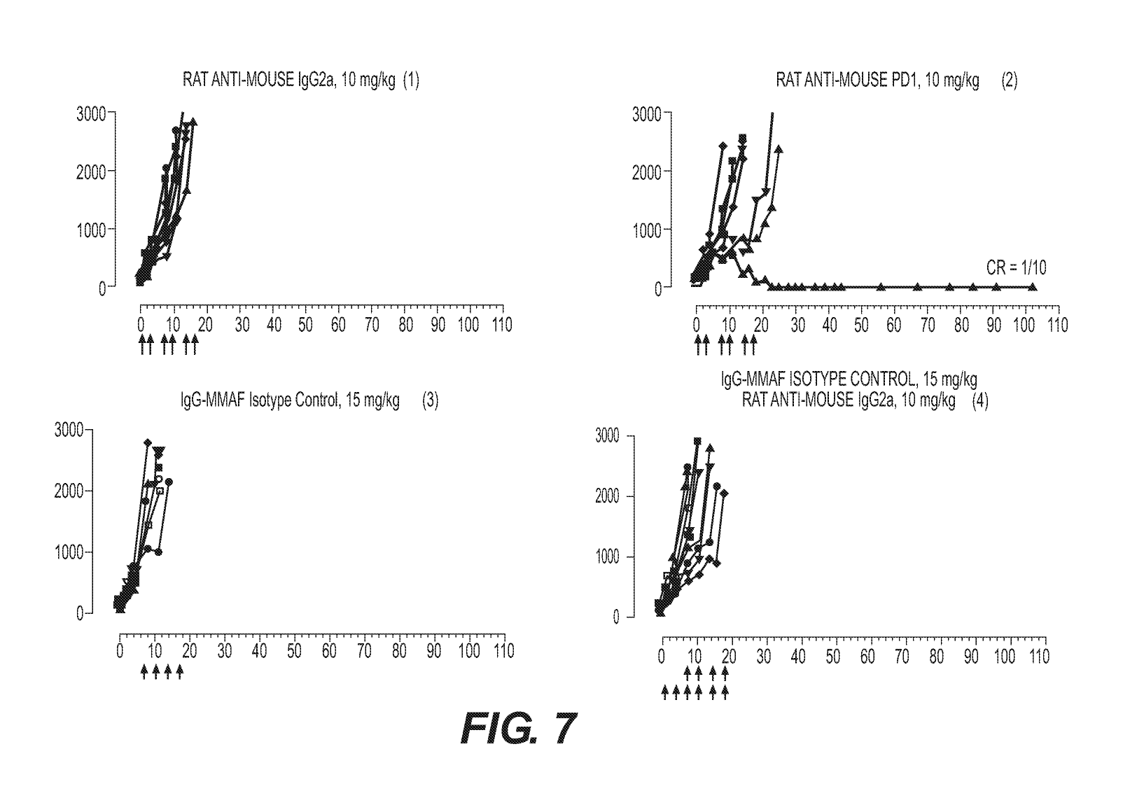

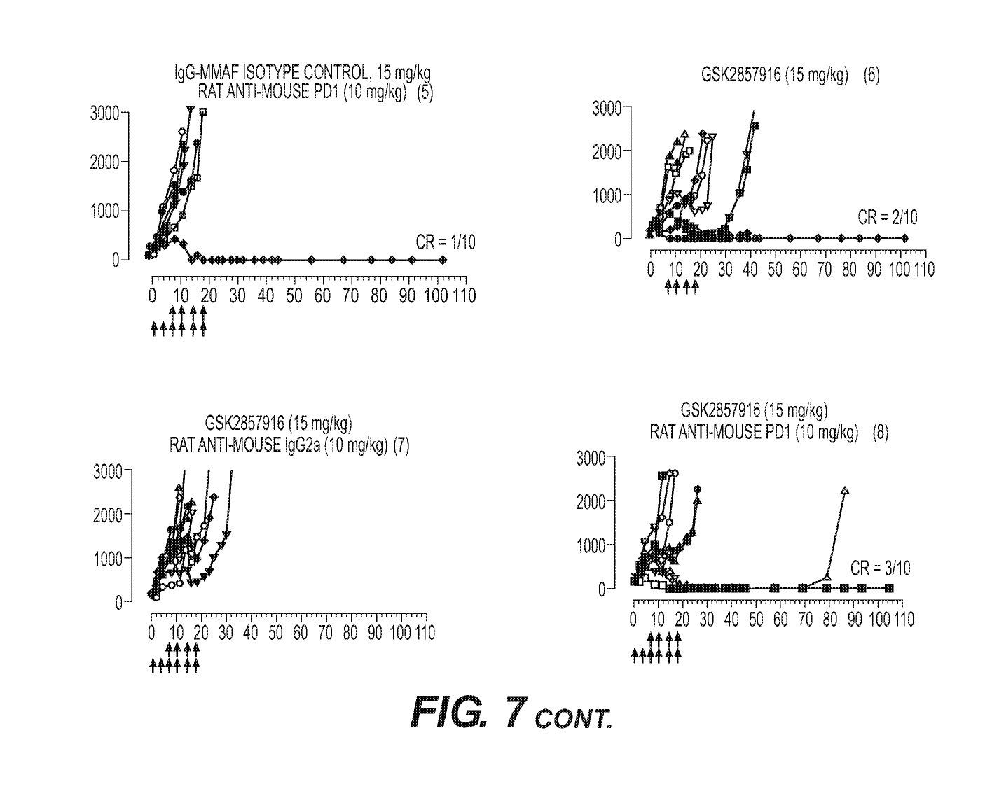

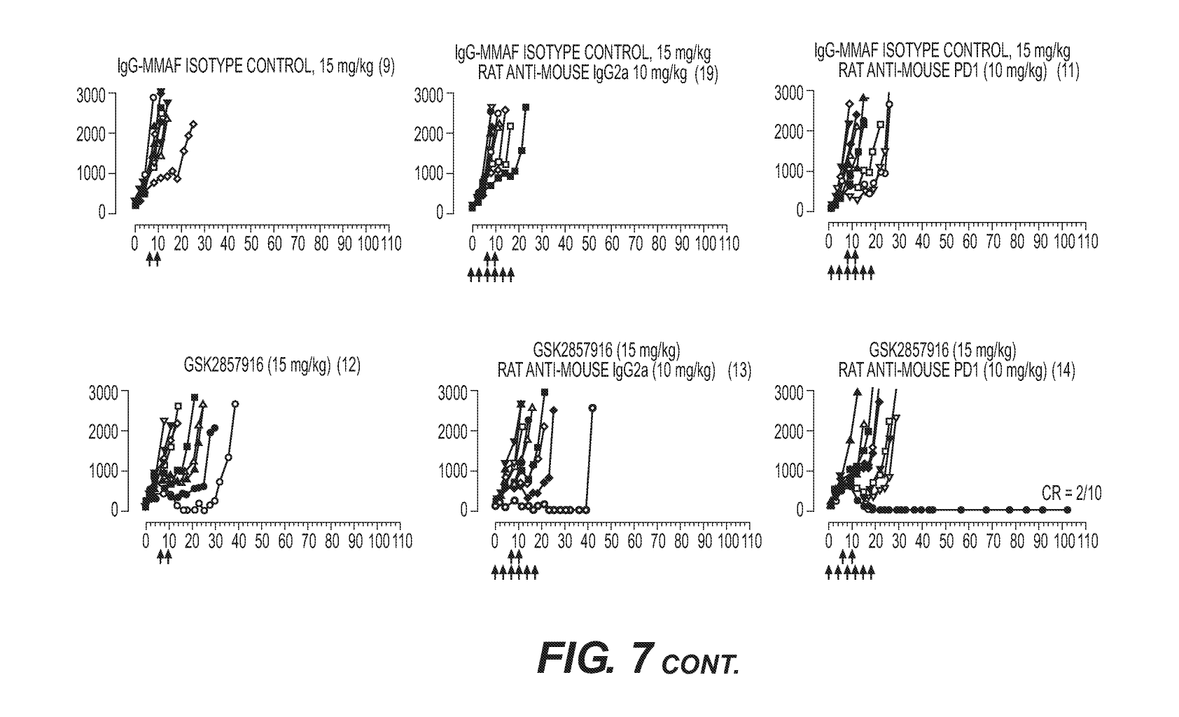

The process of immunogenic cell death can induce the production of danger molecules that lead to the activation of dendritic cells (see FIG. 1A). BCMA ADC (anti-BCMA antibody conjugated to MMAF: GSK285791) treated NCI-H929 cells produced three danger molecules (ATP, HMGB1 and CRT) upon cell killing (FIG. 1B). To investigate the effect of NCI-H929 cell killing by BCMA ADC on the activation of dendritic cells, co-culture experiments were carried out with BCMA ADC treated NCI-H929 cells and immature dendritic cells (iDCs) differentiated from human monocytes in vitro with GM-CSF/IL-4 treatment. A number of dendritic cell maturation markers as well as IL-10 and IL-12p70, two major cytokines secreted by dendritic cells upon activation, were monitored in this process to assess the effect of BCMA ADC induced cell death on dendritic cell activation.

Fresh, whole Human blood was obtained from three healthy donors from the Upper Providence Blood Donation Unit with syringes coated in liquid sodium heparin (Sagent 10 IU/mL final concentration). Monocytes were isolated from fresh, whole human blood using the RosetteSep Monocyte Enrichment kit (Cat.#15068) and were assessed for expression of cell surface CD14 (BD Bioscience Cat.#562698) using flow cytometry. Isolated Monocytes (1.5×106 cells/well) were cultured in 2 mLs of X-Vivo-15 Media supplemented with 1% autologous plasma, 50 ng/mL recombinant human GM-CSF (R&D, 215-GM-050) and 100 ng/mL recombinant human IL-4 (R&D, 204-IL-050) for 7 Days at 37C°/5% CO2. The cultures received a half media change at Day 3 or 4 while maintaining stimulation factor concentrations. NCI-H929 Multiple Myeloma cells were passaged for ≥2 passages from thaw at 37° C., 5% CO₂, 90% humid air in RPMI-1640 supplemented with 10% FBS. Cells were plated at a density of 7.5×10⁵ cells/ml in 2 mLs on 12 well plates. A dose response of J6M0 ADC enhanced antigen binding protein was added to the media and the cells were incubated at 37° C./5% CO₂ for 48 hours.

Day 7 iDCs were co-cultured with J6M0 ADC enhanced antigen binding protein treated NCI-H929 cells at 1:1 ratio for 24 hours on 96 well plates. All cells were counted at the initiation of the co-culture and diluted with X-Vivo-15 media to a concentration of 7.5×10⁵ cells/mL. 7.5×10⁴ cells/well (100 μl) each of iDCS and pre-treated NCI-H929 cells were combined in each well on a 96 well plate. Co-cultured cells were incubated 37° C./5% CO₂ for 24 hours. A flow cytometry panel consisting of CD1a, CD11c, CD40, CD80, CD83, CD86, HLA-DR, & CD14 was used to assess fresh, FcR blocked Dendritic Cell differentiation and maturation on the FACS Canto II. Additionally, cell supernatants were collected, frozen at −20° C., and assayed for IL-10 and IL-12p70 using MSD kits.

Data analysis was performed in Flow Jo v7.6.5 for the flow cytometry panel and used either CD11c+ or HLA-DR+ cell gating to distinguish the dendritic Cells from the tumor cells. NCI-H929 cells are negative for HLA-DR and have much lower CD11c expression than dendritic cells allowing the two cell populations to be distinctly gated. Data for the secreted IL-10 and IL-12p70 from supernatants in the co-culture assays w analyzed in MSD Discovery Workbench v4.0.12.

These results suggest that the killing of NCI-H929 cells with BCMA ADC has a stimulatory effect on dendritic cells and can lead to dendritic cell activation. When IL-12p70 and IL-10 were monitored, IL-12p70 was below the detection limit under all conditions. IL-10 could be detected and there was a significant inhibitory effect of BCMA ADC on 11-10 production. However, this effect may not be related to the dendritic cell activation by the immunogenic cell death of NCI-H929 cells induced by BCMA ADC since the inhibitory effect on IL-10 was observed with NCI-H929 cells alone treated with BCMA ADC. The inhibitory effect on IL-10 production is beneficial in stimulating the immune response. (See FIG. 2 and FIG. 3)

### Example 3

T cells can be activated through the engagement of the T cell receptor (TCR) and costimulatory molecules expressed on the cell surface. Upon T cell activation, a number of additional surface markers are upregulated. In vitro effect of BCMA ADC (anti-BCMA antibody conjugated to MMAF: GSK285791) on T cell activation and function was characterized by monitoring a number of these T cell activation associated markers. Furthermore, upon activation, T cells produce cytokines such as IFNγ and IL-4. The effect of BCMA ADC on IFNγ and IL-4 productions in stimulated T cells was also studied. These experiments can provide data on the effect of BCMA ADC on T cells activation and function. In addition, proliferation of human CD4+ and CD8+ T cells upon stimulation in the presence of BCMA ADC was also evaluated. Human blood was obtained from the blood donor procurement program at GlaxoSmithKline. Peripheral blood mononuclear cells (PBMCs) were isolated from human whole blood by Ficoll density gradient centrifugation (GE Healthcare). Anti-human CD3 antibody (eBioscience, catalog#16-0037-85, clone OKT3) diluted with coating buffer (Biolegend, catalog#421701) was coated on 96 well flat-bottom plate overnight.

### 3.1 Effect of BCMA ADC on T Cell Activation by Anti-CD3/Anti-CD28 Stimulation in PBMC

BCMA ADC (anti-BCMA antibody conjugated to MMAF: GSK285791) was tested in the PBMC T cell activation assay. In this study, BCMA ADC treatment and PBMC activation occurred concurrently and the effects were monitored at 24 and 72 hrs. This study was repeated two times (n=2) with blood from two different donors. In this study, 100 μL PBMCs (2×10″6 cells/mL) in RPMI-1640 with 10% FBS (Hyclone; catalog# SH30071.03) were added into anti-CD3 antibody coated wells with or without soluble 0.5 μg/mL anti-CD28 antibody (eBioscience, catalog#16-0289, clone CD28.2). A stock solution of 9.6 mg/mL BCMA ADC or 4.6 mg/mL BCMA Ab IgG control was first diluted in RPMI-1640 media to give antibody concentrations of 1 mg/mL which were further diluted in equal volume of RPMI-1640 media to give antibody concentrations of 60, 6 and 0.6 μg/mL. 100 μL each of the final diluted antibody solutions was added to 100 μL of the PBMC in RPMI-1640/10% FBS to give final antibody concentrations of 30, 3 and 0.3 μg/mL. Three technical replicates were included for each assay condition. PBMCs were cultured at 37° C. and 5% CO2 for various times as indicated above. Cells were transferred into 96-deep well plate and washed twice with 1 mL staining buffer (BD Biosciences, catalog#554656), stained with fluorescent-conjugated antibodies or isotype controls (see section 3.3. Drugs and Materials), and incubated for 30 min on ice. Immunofluorescence analysis was performed on a FACS CANTO II flow cytometer (BD Biosciences) and analyzed with DIVA software (BD Biosciences). Flow cytometry was used to monitor the percentage of CD4 or CD8 cells that express a given marker, and the mean fluorescence intensity (MFI) of this maker in CD4 or CD8 cells.

3.2 Effect of BCMA ADC on IFNγ and IL-4 production by T cells upon anti-CD3/anti-CD28 stimulation in PBMC

This study was repeated two times (n=2) with blood from two different donors. 100 μL PBMCs (1×10\hat{ }6 cells/mL) in RPMI-1640 with 10% FBS were added into anti-CD3 antibody coated wells with or without soluble 0.5 μg/mL anti-CD28 antibody. 100 μL each of the diluted BCMA ADC (anti-BCMA antibody conjugated to MMAF: GSK285791) and IgG control solutions (see section 3.1 for antibody dilution protocol) was then added and PBMCs were cultured at 37° C. and 5% CO2 for 48 hours and 72 hours. Three technical replicates were included for each assay condition. At the end of the study, cells were transferred, washed, stained and analyzed by flow cytometry as described in section 3.1. For intracellular staining, cells were fixed by cytofix buffer (BD Biosciences, Catalog#554655) at room temperature for 20 minutes and permeablized by perm/wash buffer (BD Biosciences, catalog#554723) at room temperature for 30 minutes before staining with antibodies or isotype controls.

### 3.3 Effect of BCMA ADC on T Cell Proliferation Upon Anti-CD3/Anti-CD28 Stimulation

Fresh normal peripheral blood isolated CD4+ or CD8+ T cells were received and counted using a Beckman Coulter ViCell then centrifuged at 330 g for 7 minutes. Cells were then stained with Molecular Probes Cell Trace CFSE (cat # C34554) proliferation dye (5-10 μM) in PBS/0.5% BSA. Cells were incubated for 5 minutes on ice prior to a further incubation for 5 minutes on ice in cold RPMI 1640/10% FBS. Any free dye was removed by two further cell centrifugations at 300 g for 5 minutes. Cells were resuspended in complete media RPMI 1640/10% FBS/IL-2 (2.8 ng/mL). Cells were then seeded (10⁵ cells/100 μL volume/well) in a 96 well tissue culture plates pre-coated with anti-CD3 (1 μg/mL) and anti-CD28 (1 μg/mL). BCMA ADC (anti-BCMA antibody conjugated to MMAF: GSK285791) was added to cells immediately after seeding plate and incubated at 37° C. for 96 hours.

After the 4 day incubation, cell supernatant was harvested and frozen at minus 80° C. and the cells were collected for flow cytometry staining. Analyzed using a CANTO II flow cytometer with 488 nm excitation and emission filters appropriate for fluorescein (FITC) for Cell Trace CFSE.

The antibodies and isotypes for the markers monitored by flow cytometry analysis are listed in Table 2 below.

TABLE 2

| Marker | Fluorescence | Clone | Isotype | Vendor | Catalog No. |
|---|---|---|---|---|---|
| CD4 | V450 | SK3 | mouse IgG1 | BD Biosciences | 651850 |
| CD8 | PerCP-Cy5.5 | RPA-T8 | mouse IgG1 | BioLegend | 301032 |
| CD25 | PE | BC96 | mouse IgG1 | eBioscience | 12-0259-42 |
| CD69 | PE-Cy7 | FN50 | mouse IgG1 | BioLegend | 310912 |
| PD-1 | APC | EH12.2H7 | mouse IgG1 | BioLegend | 329908 |
| OX-40 | FITC | ACT-35 | mouse IgG1 | eBioscience | 11-1347-42 |
| CTLA-4 | PE | L3D10 | mouse IgG1 | BioLegend | 349906 |
| CD39 | APC | A1 | mouse IgG1 | eBioscience | 17-0399-42 |
| CD73 | PE-Cy7 | AD2 | mouse IgG1 | BD Biosciences | 561258 |
| ICOS | FITC | Isa3 | mouse IgG1 | eBioscience | 11-9948-42 |
| CD137 | PE | 4B4-1 | mouse IgG1 | BD Biosciences | 555956 |
| LAG-3 | APC | 3DS223H | mouse IgG1 | eBioscience | 17-2239-42 |
| TIM-3 | FITC | F38-2E2 | mouse IgG1 | eBioscience | 11-3109-42 |
| CD4 | PE-Cy5 | OKT4 | mouse IgG2b | BioLegend | 317412 |
| IL-4 | PE | 8D4-8 | mouse IgG1 | BioLegend | 500704 |
| IFNγ | FITC | B27 | mouse IgG1 | BD Biosciences | 554700 |

The raw percentage and MFI data for each marker at each concentration of BCMA ADC was compared to the corresponding IgG control in the statistical analysis. To account for the variability of the different blood donors, a linear mixed effect model was used to analyze the data. Briefly, donor and the interaction between donor and group (BCMA ADC treatment or IgG control) were treated as random effects, and group was treated as a fixed effect in the model. Following the mixed effect model analysis, each BCMA ADC treatment group was then compared with the IgG control group. Due to multiple comparisons, Dunnett's method was used to control the overall Type-1 error rate. The adjusted p-values 0.05 by Dunnett's method were declared to be significant for the percentage or MFI value at a specific BCMA ADC concentration. BCMA ADC was considered to significantly change the expression of a marker if at least 2 out of the 3 concentrations of BCMA ADC induced a statistically significant (p-values 0.05) change of the percentage or MFI value of the marker.

For data reporting, the average percentage or average MFI value among three technical replicates was generated at each BCMA ADC concentration and the % difference values were calculated as: % difference=(Avg 916−Avg IgG)*100/Avg IgG, where Avg 916 and Avg IgG represent the average values of the BCMA ADC treatment group and the IgG control group, respectively. The % difference values with different donors for a given marker at a given BCMA ADC concentration and time point were used to calculate the average % difference value and CV (coefficient of variation) which were reported in FIG. 4A-C. Positive and negative % difference values represent upregulation and downregulation of a marker, respectively.

CD4% and CD8% were measured independently in three groups for each experiment, with three technical replicates in each group. Each group was analyzed statistically as described above. Changes in CD4% and CD8% values were considered significant if at least 2 out of the 3 groups had significant changes (p-values 0.05). For data reporting, a pooled CV was generated by taking the average of CVs for 3 groups.

In this study, in vitro effect of BCMA ADC on T cell activation and function was characterized by monitoring the effect of compound on a number of T cell activation associated markers. Majority of these markers are upregulated upon T cell activation. These markers include T cell activation markers CD25 and CD69; co-inhibitory markers PD-1, CTLA-4; costimulatory markers ICOS, OX-40 and CD137; and T cell exhaustion markers TIM3 and LAG3. CD73 and CD39 are surface proteins involved in the adenosine pathway activation and are considered as co-inhibitory molecules in T cell activation. The correlation between their expression level and T cell activation is less well understood. Overall, the data indicate that BCMA ADC has minimal effect on anti-CD3/anti-CD28 stimulated CD4 and CD8 T cell activation in PBMC. BCMA ADC has no significant effect on IFNγ and IL-4 production in both CD4 and CD8 cells in PBMC after anti-CD3/anti-CD28 stimulation. These data are consistent with the lack of BCMA expression on human T cells.

### Example 4: In Vivo Efficacy of BCMA ADC in Combination with Anti-OX40 Antibody

All procedures on animals were reviewed and approved by the GSK Institutional Animal Care and Use Committee prior to initiation of the studies.

### 4.1 Syngeneic EL4-Luc2-hBCMA Mouse Model

The purpose of these experiments was to evaluate the combinations described herein in mouse syngeneic tumorgenesis models. EL4-Luc2 (Bioware Ultra EL4-luc2 #58230040) was transfected with a plasmid encoding human BCMA. EL4 is a mouse lymphoma cell induced in a C57BL mouse by DBMA (ATCC TIB-39). At day −13, C57BL/6 female mice (n=10) were weighed and inoculated into the right flank of with 1×10⁵ of the transduced EL4-Luc2-hBCMA cells and allowed to grow until tumor volume reached ˜700 mm³. Tumor growth was measured using a Fowler “ProMax” digital caliper. Length (L) and width (W) of tumors were measured in order to determine tumor volume using the formula: Tumor Volume=0.52×L×W².

### 4.2 Dosing Regimen

At Day 0 when target tumor volume was achieved, the mice were randomized into 12 treatment groups. Treatments were administered at Day 4, Day 7, Day 11, and Day 15. Tumor volume and body weight was measured starting at Day 0 through Day 27 at 3 times per week. Animals were euthanized when a tumor reached a volume of 2000 mm³. The dosing regimen is summarized in Table 3.

TABLE 3

| | | | Treatment Days | | | |
|---|---|---|---|---|---|---|
| | | | Day | Day | Day | Day |
| Group | Treatment | (mg/kg) | 4 | 7 | 11 | 15 |
| 1 | Saline | | X | X | X | |
| 2 | Rat IgG1 | 5 | X | X | X | |
| 3 | MMAF-human IgG1 | 15 | X | X | X | X |
| 4 | anti-mouse OX40 mAb | 1 | X | X | X | |
| | (BioCell #BE0031) | | | | | |
| 4 | MMAF-human IgG1 | 15 | X | X | X | X |
| 5 | anti-mouse OX40 mAb | 5 | X | X | X | |
| | (BioCell #BE0031) | | | | | |
| 5 | MMAF-human IgG1 | 15 | X | X | X | X |
| 6 | anti-mouse OX40 mAb | 0.25 | X | X | X | |
| | (BioCell #BE0031) | | | | | |
| 6 | MMAF-human IgG1 | 15 | X | X | X | X |
| 7 | Rat IgG1 | 5 | X | X | X | |
| 7 | MMAF-human IgG1 | 15 | X | X | X | X |
| 8 | GSK2857916 | 15 | X | X | X | X |
| 9 | Rat IgG1 | 5 | X | X | X | |
| 9 | GSK2857916 | 15 | X | X | X | X |
| 10 | anti-mouse OX40 mAb | 5 | X | X | X | |
| | (BioCell #BE0031) | | | | | |
| 10 | GSK2857916 | 15 | X | X | X | X |
| 11 | anti-mouse OX40 mAb | 1 | X | X | X | |
| | (BioCell #BE0031) | | | | | |
| 11 | GSK2857916 | 15 | X | X | X | X |
| 12 | anti-mouse OX40 mAb | 0.25 | X | X | X | |
| | (BioCell #BE0031) | | | | | |
| 12 | GSK2857916 | 15 | X | X | X | X |

### 4.3 Results

The results of Example 4 are reproduced in FIG. 5. FIG. 5A represents tumor volume. The X axis represents the number of days in the study and the Y axis represents tumor volume (mm³). Each line in a single graph in FIG. 5A represents a single mouse (n=10 mice for each treatment group). FIG. 5B represents the overall survival rate. These results demonstrate that tumor volume shrinkage is modestly enhanced when a BCMA ACD (anti-BCMA antibody conjugated to MMAF: GSK285791) is combined with an anti-OX40 antibody.

### Example 5: In Vivo Efficacy of Anti-BCMA Antibody in Combination with Anti-PD-1 Antibody

All procedures on animals were reviewed and approved by the GSK Institutional Animal Care and Use Committee prior to initiation of the studies.

### 5.1 Syngeneic EL4-Luc2-hBCMA Mouse Model

The same EL4-Luc2-hBCMA mouse model was used as described in Example 4.

### 5.2 Dosing Regimen

At Day 0 when tumor volume reached an average of 200 mm³, the mice were randomized into 13 treatment groups. Treatments were administered on Day 0, Day 4, Day 8, Day 11, Day 15, and Day 17. Treatments days and dosing schedule is summarized in Table 4. Tumor volume and body weight was measured starting at Day 0 through Day 57. Animals were euthanized when a tumor reached a volume of 2000 mm³. The dosing regimen is summarized in Table 4.

TABLE 4

| | | | Treatment Days | | | | | |
|---|---|---|---|---|---|---|---|---|
| Group | Treatment | (mg/kg) | Day 0 | Day 4 | Day 8 | Day 11 | Day 15 | Day 17 |
| 1 | Saline | | X | X | X | X | X | X |
| 2 | Rat anti-mouse IgG2a | 10 | X | X | X | X | X | X |
| 3 | IgG-MMAF | 15 | X | X | X | X | X | X |
| 4 | IgG-MMAF | 15 | X | X | X | X | X | X |
| 4 | Rat anti-mouse IgG2a | 10 | X | X | X | X | X | X |
| 5 | Rat anti-mouse PD-1 | 10 | X | X | X | X | X | X |
| | (BioCel #BE0146) | | | | | | | |
| 6 | GSK2857916 | 10 | X | X | X | X | X | X |
| 7 | GSK2857916 | 10 | X | X | X | X | X | X |
| 7 | Rat anti-mouse PD-1 | 10 | X | X | X | X | X | X |
| | (BioCel #BE0146) | | | | | | | |
| 8 | GSK2857916 | 15 | X | X | X | X | X | X |
| 9 | GSK2857916 | 15 | X | X | X | X | X | X |
| 9 | Rat anti-mouse PD-1 | 10 | | | X | X | X | X |
| | (BioCel #BE0146) | | | | | | | |
| 10 | GSK2857916 | 15 | | | X | X | X | X |
| 10 | Rat anti-mouse PD-1 | 10 | X | X | X | X | X | X |
| | (BioCel #BE0146) | | | | | | | |
| 11 | GSK2857916 | 15 | X | X | X | X | X | X |
| 11 | Rat anti-mouse PD-1 | 10 | X | X | X | X | X | X |
| | (BioCel #BE0146) | | | | | | | |
| 12 | GSK2857916 | 15 | X | X | | | | |
| 12 | Rat anti-mouse PD-1 | 10 | | | X | X | X | X |
| | (BioCel #BE0146) | | | | | | | |
| 13 | GSK2857916 | 15 | | | X | X | X | X |
| 13 | Rat anti-mouse PD-1 | 10 | X | X | | | | |
| | (BioCel #BE0146) | | | | | | | |

### 5.3 Results

The resulting tumor volumes for Example 5 are represented in FIG. 6. The X axis represents the number of days in the study and the Y axis represents tumor volume (mm³). Each line in a single graph in FIG. 6 represents a single mouse (n=10 mice for each treatment group).

### Example 6: In Vivo Efficacy of Combinations

All procedures on animals were reviewed and approved by the GSK Institutional Animal Care and Use Committee prior to initiation of the studies.

### 6.1 Syngeneic EL4-Luc2-hBCMA Mouse Model

The same EL4-Luc2-hBCMA mouse model was used as described in Example 4.

### 6.2 Dosing Regimen

At Day 0 when target tumor volume was achieved, the mice were randomized into 14 treatment groups. Treatments were administered at Day 0, Day 3, Day 7, Day 10, Day 14, and Day 17. Tumor volume and body weight was measured starting at Day 0 through Day 102. Animals were euthanized when a tumor reached a volume of 2000 mm³. The dosing regimen is summarized in Table 4. The dosing regimen is summarized in Table 5.

TABLE 5

| | | | Treatment Days | | | | | |
|---|---|---|---|---|---|---|---|---|
| Group | Treatment | (mg/kg) | 0 | 3 | 7 | 10 | 14 | 17 |
| 1 | Rat anti-mouse | 10 | X | X | X | X | X | X |
| | IgG2a | | | | | | | |
| 2 | Rat anti-mouse PD-1 | 10 | X | X | X | X | X | X |
| | (BioCel #BE0146) | | | | | | | |
| 3 | MMAF human IgG1 | 15 | | | X | X | X | X |
| 4 | MMAF human IgG1 | 15 | | | X | X | X | X |
| 4 | Rat anti-mouse | 10 | X | X | X | X | X | X |
| | IgG2a | | | | | | | |
| 5 | MMAF human IgG1 | 15 | | | X | X | X | X |
| 5 | Rat anti-mouse PD-1 | 10 | X | X | X | X | X | X |
| | (BioCel #BE0146) | | | | | | | |
| 6 | GSK2857916 | 15 | | | X | X | X | X |
| 7 | GSK2857916 | 15 | | | X | X | X | X |
| 7 | Rat anti-mouse | 10 | X | X | X | X | X | X |
| | IgG2a | | | | | | | |
| 8 | GSK2857916 | 15 | | | X | X | X | X |
| 8 | Rat anti-mouse PD-1 | 10 | X | X | X | X | X | X |
| | (BioCel #BE0146) | | | | | | | |
| 9 | MMAF human IgG1 | 15 | | | X | X | | |
| 10 | MMAF human IgG1 | 15 | | | X | X | | |
| 10 | Rat anti-mouse | 10 | X | X | X | X | X | X |
| | IgG2a | | | | | | | |
| 11 | MMAF human IgG1 | 15 | | | X | X | | |
| 11 | Rat anti-mouse PD-1 | 10 | X | X | X | X | X | X |
| | (BioCel #BE0146) | | | | | | | |
| 12 | GSK2857916 | 15 | | | X | X | | |
| 13 | GSK2857916 | 15 | | | X | X | | |
| 13 | Rat anti-mouse | 10 | X | X | X | X | X | X |
| | IgG2a | | | | | | | |
| 14 | GSK2857916 | 15 | | | X | X | | |
| 14 | Rat anti-mouse PD-1 | 10 | X | X | X | X | X | X |
| | (BioCel #BE0146) | | | | | | | |

### 6.3 Results

The resulting tumor volumes for Example 6 are represented by the graphs in FIG. 7. The X axis represents the number of days in the study and the Y axis represents tumor volume (mm³). Each line in a single graph in FIG. 7 represents a single mouse (n=10 mice for each treatment group). The results demonstrate that treatment with a combination of a BCMA ADC (anti-BCMA antibody conjugated to MMAF: GSK285791) and an anti-PD-1 antibody results in a moderate and consistent reduction in tumor volume compared to treatment with a BCMA ADC (anti-BCMA antibody conjugated to MMAF: GSK285791) alone or an anti-PD-1 antibody alone.

TABLE A
Sequence Summary

| Description | Amino acid sequence | Polynucleotide sequence |
|---|---|---|
| CA8 CDRH1 | SEQ. I.D. NO: 1 | n/a |
| CA8 CDRH2 | SEQ. I.D. NO: 2 | n/a |
| CA8 CDRH3 | SEQ. I.D. NO: 3 | n/a |
| CA8 CDRL1 | SEQ. I.D. NO: 4 | n/a |
| CA8 CDRL2 | SEQ. I.D. NO: 5 | n/a |
| CA8 CDRL3 | SEQ. I.D. NO: 6 | n/a |
| CA8 V_{H} domain (murine) | SEQ. I.D. NO: 7 | SEQ. I.D. NO: 8 |
| CA8 V_{L} domain (murine) | SEQ. I.D. NO: 9 | SEQ. I.D. NO: 10 |
| CA8 Humanised V_{H} J0 | SEQ. I.D. NO: 11 | SEQ. I.D. NO: 12 |
| CA8 Humanised V_{H} J1 | SEQ. I.D. NO: 13 | SEQ. I.D. NO: 14 |
| CA8 Humanised V_{H} J2 | SEQ. I.D. NO: 15 | SEQ. I.D. NO: 16 |
| CA8 Humanised V_{H} J3 | SEQ. I.D. NO: 17 | SEQ. I.D. NO: 18 |
| CA8 Humanised V_{H} J4 | SEQ. I.D. NO: 19 | SEQ. I.D. NO: 20 |
| CA8 Humanised V_{H} J5 | SEQ. I.D. NO: 21 | SEQ. I.D. NO: 22 |
| CA8 Humanised V_{H} J6 | SEQ. I.D. NO: 23 | SEQ. I.D. NO: 24 |
| CA8 Humanised V_{H} J7 | SEQ. I.D. NO: 25 | SEQ. I.D. NO: 26 |
| CA8 Humanised V_{H} J8 | SEQ. I.D. NO: 27 | SEQ. I.D. NO: 28 |
| CA8 Humanised V_{H} J9 | SEQ. I.D. NO: 29 | SEQ. I.D. NO: 30 |
| CA8 Humanised V_{L} M0 | SEQ. I.D. NO: 31 | SEQ. I.D. NO: 32 |
| CA8 Humanised V_{L} M1 | SEQ. I.D. NO: 33 | SEQ. I.D. NO: 34 |
| CA8 Humanised V_{L} M2 | SEQ. I.D. NO: 35 | SEQ. I.D. NO: 36 |
| Human BCMA | SEQ. I.D. NO: 37 | SEQ. I.D. NO: 38 |
| CD33-hBCMA ECD (1-53) TEV-Fc | | |
| Human BCMA | SEQ. I.D. NO: 39 | SEQ. I.D. NO: 40 |
| CD33-hBCMA ECD (4-53) TEV-Fc | | |
| Cyno BCMA | SEQ. I.D. NO: 41 | SEQ. I.D. NO: 42 |
| CD33 cyno BCMA ECD (4-52) TEV-Fc | | |
| CA8 J0 Humanised heavy chain | SEQ. I.D. NO: 43 | SEQ. I.D. NO: 44 |
| CA8 J1 Humanised heavy chain | SEQ. I.D. NO: 45 | SEQ. I.D. NO: 46 |
| CA8 J2 Humanised heavy chain | SEQ. I.D. NO: 47 | SEQ. I.D. NO: 48 |
| CA8 J3 Humanised heavy chain | SEQ. I.D. NO: 49 | SEQ. I.D. NO: 50 |
| CA8 J4 Humanised heavy chain | SEQ. I.D. NO: 51 | SEQ. I.D. NO: 52 |
| CA8 J5 Humanised heavy chain | SEQ. I.D. NO: 53 | SEQ. I.D. NO: 54 |
| CA8 J6 Humanised heavy chain | SEQ. I.D. NO: 55 | SEQ. I.D. NO: 56 |
| CA8 J7 Humanised heavy chain | SEQ. I.D. NO: 57 | SEQ. I.D. NO: 58 |
| CA8 J8 Humanised heavy chain | SEQ. I.D. NO: 59 | SEQ. I.D. NO: 60 |
| CA8 J9 Humanised heavy chain | SEQ. I.D. NO: 61 | SEQ. I.D. NO: 62 |
| CA8 M0 Humanised light chain | SEQ. I.D. NO: 63 | SEQ. I.D. NO: 64 |
| CA8 M1 Humanised light chain | SEQ. I.D. NO: 65 | SEQ. I.D. NO: 66 |
| CA8 M2 Humanised light chain | SEQ. I.D. NO: 67 | SEQ. I.D. NO: 68 |
| S307118G03 V_{H} domain (murine) | SEQ. I.D. NO: 69 | SEQ. I.D. NO: 70 |
| S307118G03 V_{L} domain (murine) | SEQ. I.D. NO: 71 | SEQ. I.D. NO: 72 |
| S307118G03 heavy chain (chimeric) | SEQ. I.D. NO: 73 | SEQ. I.D. NO: 74 |
| S307118G03 light chain(chimeric) | SEQ. I.D. NO: 75 | SEQ. I.D. NO: 76 |
| S307118G03 Humanised V_{H} H0 | SEQ. I.D. NO: 77 | SEQ. I.D. NO: 78 |
| S307118G03 Humanised V_{H} H1 | SEQ. I.D. NO: 79 | SEQ. I.D. NO: 80 |
| S307118G03 humanised V_{H} H2 | SEQ. I.D. NO: 81 | SEQ. I.D. NO: 82 |
| S307118G03 humanised V_{H} H3 | SEQ. I.D. NO: 83 | SEQ. I.D. NO: 84 |
| S307118G03 humanised V_{H} H4 | SEQ. I.D. NO: 85 | SEQ. I.D. NO: 86 |
| S307118G03 humanised V_{H} H5 | SEQ. I.D. NO: 87 | SEQ. I.D. NO: 88 |
| S307118G03 humanised V_{L} L0 | SEQ. I.D. NO: 89 | SEQ. I.D. NO: 90 |
| S307118G03 humanised V_{L} L1 | SEQ. I.D. NO: 91 | SEQ. I.D. NO: 92 |
| S307118G03 CDRH1 | SEQ. I.D. NO: 93 | n/a |
| S307118G03 CDRH2 | SEQ. I.D. NO: 94 | n/a |
| S307118G03 CDRH3 | SEQ. I.D. NO: 95 | n/a |
| S307118G03 CDRL1 | SEQ. I.D. NO: 96 | n/a |
| S307118G03 CDRL2 | SEQ. I.D. NO: 97 | n/a |
| S307118G03 CDRL3 | SEQ. I.D. NO: 98 | n/a |
| S307118G03 humanised H5 CDRH3 | SEQ. I.D. NO: 99 | n/a |
| S307118G03 H0 Humanised heavy chain | SEQ. I.D. NO: 100 | SEQ. I.D. NO: 101 |
| S307118G03 H1 humanised heavy chain | SEQ. I.D. NO: 102 | SEQ. I.D. NO: 103 |
| S307118G03 H2 humanised heavy chain | SEQ. I.D. NO: 104 | SEQ. I.D. NO: 105 |
| S307118G03 H3 humanised heavy chain | SEQ. I.D. NO: 106 | SEQ. I.D. NO: 107 |
| S307118G03 H4 humanised heavy chain | SEQ. I.D. NO: 108 | SEQ. I.D. NO: 109 |
| S307118G03 H5 humanised heavy chain | SEQ. I.D. NO: 110 | SEQ. I.D. NO: 111 |
| S307118G03 L0 humanised light chain | SEQ. I.D. NO: 112 | SEQ. I.D. NO: 113 |
| S307118G03 L1 humanised light chain | SEQ. I.D. NO: 114 | SEQ. I.D. NO: 115 |
| S332121F02 murine variable heavy chain | SEQ. I.D. NO: 116 | SEQ. I.D. NO: 117 |
| S332121F02 chimeric variable heavy chain | SEQ. I.D. NO: 118 | SEQ. I.D. NO: 119 |
| S332121F02 murine variable light chain | SEQ. I.D. NO: 120 | SEQ. I.D. NO: 121 |
| S332121F02 chimeric variable light chain | SEQ. I.D. NO: 122 | SEQ. I.D. NO: 123 |
| S322110D07 murine variable heavy chain | SEQ. I.D. NO: 124 | SEQ. I.D. NO: 125 |
| S322110D07 chimeric heavy chain | SEQ. I.D. NO: 126 | SEQ. I.D. NO: 127 |
| S322110D07 murine variable light chain | SEQ. I.D. NO: 128 | SEQ. I.D. NO: 129 |
| S322110D07 chimeric light chain | SEQ. I.D. NO: 130 | SEQ. I.D. NO: 131 |
| S332126E04 murine variable heavy chain | SEQ. I.D. NO: 132 | SEQ. I.D. NO: 133 |
| S332126E04 Chimeric heavy chain | SEQ. I.D. NO: 134 | SEQ. I.D. NO: 135 |
| S332126E04 murine variable light chain | SEQ. I.D. NO: 136 | SEQ. I.D. NO: 137 |
| S332126E04 Chimeric light chain | SEQ. I.D. NO: 138 | SEQ. I.D. NO: 139 |
| S336105A07 murine variable heavy chain | SEQ. I.D. NO: 140 | SEQ. I.D. NO: 141 |
| S336105A07 Chimeric heavy chain | SEQ. I.D. NO: 142 | SEQ. I.D. NO: 143 |
| S336105A07 murine variable light chain | SEQ. I.D. NO: 144 | SEQ. I.D. NO: 145 |
| S336105A07 chimeric light chain | SEQ. I.D. NO: 146 | SEQ. I.D. NO: 147 |
| S335115G01 murine variable heavy chain | SEQ. I.D. NO: 148 | SEQ. I.D. NO: 149 |
| S335115G01 Chimeric heavy chain | SEQ. I.D. NO: 150 | SEQ. I.D. NO: 151 |
| S335115G01 murine variable light chain | SEQ. I.D. NO: 152 | SEQ. I.D. NO: 153 |
| S335115G01 Chimeric light chain | SEQ. I.D. NO: 154 | SEQ. I.D. NO: 155 |
| S335122F05 murine variable heavy chain | SEQ. I.D. NO: 156 | SEQ. I.D. NO: 158 |
| S335122F05 Chimeric heavy chain | SEQ. I.D. NO: 158 | SEQ. I.D. NO: 159 |
| S335122F05 murine variable light chain | SEQ. I.D. NO: 160 | SEQ. I.D. NO: 161 |
| S335122F05 Chimeric light chain | SEQ. I.D. NO: 162 | SEQ. I.D. NO: 163 |
| S332121F02 CDRH1 | SEQ. I.D. NO: 164 | n/a |
| S332121F02 CDRH2 | SEQ. I.D. NO: 165 | n/a |
| S332121F02 CDRH3 | SEQ. I.D. NO: 166 | n/a |
| S332121F02 CDRL1 | SEQ. I.D. NO: 167 | n/a |
| S332121F02 CDRL2 | SEQ. I.D. NO: 168 | n/a |
| S332121F02 CDRL3 | SEQ. I.D. NO: 169 | n/a |
| S322110D07 CDRH1 | SEQ. I.D. NO: 170 | n/a |
| S322110D07 CDRH2 | SEQ. I.D. NO: 171 | n/a |
| S322110D07 CDRH3 | SEQ. I.D. NO: 172 | n/a |
| S322110D07CDRL1 | SEQ. I.D. NO: 173 | n/a |
| S322110D07 CDRL2 | SEQ. I.D. NO: 174 | n/a |
| S322110D07 CDRL3 | SEQ. I.D. NO: 175 | n/a |
| S332126E04CDRH1 | SEQ. I.D. NO: 176 | n/a |
| S332126E04 CDRH2 | SEQ. I.D. NO: 177 | n/a |
| S332126E04 CDRH3 | SEQ. I.D. NO: 178 | n/a |
| S332126E04 CDRL1 | SEQ. I.D. NO: 179 | n/a |
| S332126E04 CDRL2 | SEQ. I.D. NO: 180 | n/a |
| S332126E04 CDRL3 | SEQ. I.D. NO: 181 | n/a |
| S336105A07 CDRH1 | SEQ. I.D. NO: 182 | n/a |
| S336105A07 CDRH2 | SEQ. I.D. NO: 183 | n/a |
| S336105A07 CDRH3 | SEQ. I.D. NO: 184 | n/a |
| S336105A07 CDRL1 | SEQ. I.D. NO: 185 | n/a |
| S336105A07 CDRL2 | SEQ. I.D. NO: 186 | n/a |
| S336105A07 CDRL3 | SEQ. I.D. NO: 187 | n/a |
| S335115G01 CDRH1 | SEQ. I.D. NO: 188 | n/a |
| S335115G01 CDRH2 | SEQ. I.D. NO: 189 | n/a |
| S335115G01 CDRH3 | SEQ. I.D. NO: 190 | n/a |
| S335115G01 CDRL1 | SEQ. I.D. NO: 191 | n/a |
| S335115G01 CDRL2 | SEQ. I.D. NO: 192 | n/a |
| S335115G01 CDRL3 | SEQ. I.D. NO: 193 | n/a |
| S335122F05 CDRH1 | SEQ. I.D. NO: 194 | n/a |
| S335122F05 CDRH2 | SEQ. I.D. NO: 195 | n/a |
| S335122F05 CDRH3 | SEQ. I.D. NO: 196 | n/a |
| S335122F05 CDRL1 | SEQ. I.D. NO: 197 | n/a |
| S335122F05 CDRL2 | SEQ. I.D. NO: 198 | n/a |
| S335122F05 CDRL3 | SEQ. I.D. NO: 199 | n/a |
| CA8 CDRH3 variant N99D | SEQ. I.D. NO: 200 | n/a |
| Pembrolizumab CDRH1 | SEQ. I.D. NO: 201 | n/a |
| Pembrolizumab CDRH2 | SEQ. I.D. NO: 202 | n/a |
| Pembrolizumab CDRH3 | SEQ. I.D. NO: 203 | n/a |
| Pembrolizumab CDRL1 | SEQ. I.D. NO: 204 | n/a |
| Pembrolizumab CDRL2 | SEQ. I.D. NO: 205 | n/a |
| Pembrolizumab CDRL3 | SEQ. I.D. NO: 206 | n/a |
| Pembrolizumab variable heavy chain (VH) | SEQ. I.D. NO: 207 | n/a |
| Pembrolizumab variable light chain (VL) | SEQ. I.D. NO: 208 | n/a |
| Pembrolizumab Heavy chain | SEQ. I.D. NO: 209 | n/a |
| Pembrolizumab Light chain | SEQ. I.D. NO: 210 | n/a |
| Nivolumab CDRH1 | SEQ. I.D. NO: 211 | n/a |
| Nivolumab CDRH2 | SEQ. I.D. NO: 212 | n/a |
| Nivolumab CDRH3 | SEQ. I.D. NO: 213 | n/a |
| Nivolumab CDRL1 | SEQ. I.D. NO: 214 | n/a |
| Nivolumab CDRL2 | SEQ. I.D. NO: 215 | n/a |
| Nivolumab CDRL3 | SEQ. I.D. NO: 216 | n/a |
| Nivolumab variable heavy chain (VH) | SEQ. I.D. NO: 217 | n/a |
| Nivolumab variable light chain (VL) | SEQ. I.D. NO: 218 | n/a |
| 106-222 CDRH1 | SEQ. I.D. NO: 219 | n/a |
| 106-222 CDRH2 | SEQ. I.D. NO: 220 | n/a |
| 106-222 CDRH3 | SEQ. I.D. NO: 221 | n/a |
| 106-222 CDRL1 | SEQ. I.D. NO: 222 | n/a |
| 106-222 CDRL2 | SEQ. I.D. NO: 223 | n/a |
| 106-222 CDRL3 | SEQ. I.D. NO: 224 | n/a |
| 106-222 Variable heavy chain | SEQ. I.D. NO: 225 | SEQ. I.D. NO: 226 |
| 106-222 Variable light chain | SEQ. I.D. NO: 227 | SEQ. I.D. NO: 228 |
| 106-222 Variable heavy chain | SEQ. I.D. NO: 229 | n/a |
| 106-222 Variable light chain | SEQ. I.D. NO: 230 | n/a |
| 119-222 CDRH1 | SEQ. I.D. NO: 231 | n/a |
| 1119-222 CDRH2 | SEQ. I.D. NO: 232 | n/a |
| 119-222 CDRH3 | SEQ. I.D. NO: 233 | n/a |
| 119-222 CDRL1 | SEQ. I.D. NO: 234 | n/a |
| 119-222 CDRL2 | SEQ. I.D. NO: 235 | n/a |
| 119-222 CDRL3 | SEQ. I.D. NO: 236 | n/a |
| 119-222 Variable heavy chain | SEQ. I.D. NO: 237 | SEQ. I.D. NO: 238 |
| 119-222 Variable light chain | SEQ. I.D. NO: 239 | SEQ. I.D. NO: 240 |
| 119-222 Variable heavy chain | SEQ. I.D. NO: 241 | n/a |
| 119-222 Variable light chain | SEQ. I.D. NO: 242 | n/a |
| 106-222 Heavy Chain | SEQ. I.D. NO: 243 | n/a |
| 106-222 Light Chain | SEQ. I.D. NO: 244 | n/a |

1. A combination comprising a therapeutically effective amount of an antigen binding protein that binds BCMA and a therapeutically effective amount of at least one antigen binding protein that binds an immunomodulatory target.

2. The combination of claim 1, wherein the immunomodulatory target is PD1 or OX40.

3. The combination of claim 1, wherein the antigen binding protein that binds BCMA is conjugated to a cytotoxic agent as an immunoconjugate, and wherein the cytotoxic agent is MMAE or MMAF.

4. The combination of claim 3, wherein the cytotic agent is conjugated to the antigen binding protein that binds BCMA via a linker, and wherein the linker is citruline-valine or maleimidocaproyl.

5. The combination of claim 1, wherein the antigen binding protein that binds BCMA comprises CDRH3 variant N99D of SEQ. ID. NO: 200.

6. The combination of claim 1, wherein the antigen binding protein that binds BCMA comprises CDRH3 of SEQ. ID. NO: 3, or a variant thereof.

7. The combination of claim 1, wherein the antigen binding protein that binds BCMA comprises CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, and CDRL3 of SEQ. ID. NO: 6.

8. The combination of claim 1, wherein the antigen binding protein that binds BCMA comprises heavy chain variable region of SEQ. ID. NO: 23 and light chain variable region of SEQ. ID. NO: 31.

9. The combination of claim 2, wherein the antigen binding protein that binds PD-1 is pembrolizumab or nivolumab.

10. The combination of claim 2, wherein the antigen binding protein that binds OX40 comprises CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, and CDRL3 of SEQ. ID. NO: 224.

11. The combination of claim 2 wherein the antigen binding protein that binds OX40 comprises heavy chain variable region of SEQ. ID. NO: 229 and light chain variable region of SEQ. ID. NO: 230.

12. A combination comprising:
i) an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID.NO: 200, CDRL1 of SEQ. ID. NO: 4, CDRL2 of SEQ. ID. NO: 5, and CDRL3 of SEQ. ID. NO: 6 conjugated to MMAF; and
ii) pembrolizumab or nivolumab.

13. A combination comprising:
i) an antigen binding protein that binds BCMA, wherein the antigen binding protein that binds BCMA is an immunoconjugate comprising an anti-BCMA antibody comprising CDRH1 of SEQ. ID. NO: 1, CDRH2 of SEQ. ID. NO: 2, CDRH3 of SEQ. ID. NO: 200, CDRL1 of SEQ. ID. NO: 4, CDRL2 of SEQ. ID. NO: 5, and CDRL3 of SEQ. ID. NO: 6 conjugated to MMAF; and
ii) an antigen binding protein that binds OX40 comprising CDRH1 of SEQ. ID. NO: 219, CDRH2 of SEQ. ID. NO: 220, CDRH3 of SEQ. ID. NO: 221, CDRL1 of SEQ. ID. NO: 222, CDRL2 of SEQ. ID. NO: 223, and CDRL3 of SEQ. ID. NO: 224.

14. A pharmaceutical composition comprising the combination of claim 1.

15. A method of treating cancer in a mammal in need thereof comprising administering a therapeutically effective amount of the pharmaceutical composition of claim 14.

16. The method of claim 15, wherein the immunomodulatory target is PD1 or OX40.

17. (canceled)

18. The method of claim 15, wherein the cancer is multiple myeloma or non-Hodgkins lymphoma.

19.-22. (canceled)


SEQUENCE LISTING (244 sequences)

SEQ ID NO: 1 - protein, 5 aa, Mus musculus
NYWMH

SEQ ID NO: 2 - protein, 17 aa, Mus musculus
ATYRGHSDTYYNQKFKG

SEQ ID NO: 3 - protein, 12 aa, Mus musculus
GAIYNGYDVLDN

SEQ ID NO: 4 - protein, 11 aa, Mus musculus
SASQDISNYLN

SEQ ID NO: 5 - protein, 7 aa, Mus musculus
YTSNLHS

SEQ ID NO: 6 - protein, 9 aa, Mus musculus
QQYRKLPWT

SEQ ID NO: 7 - protein, 121 aa, Mus musculus
EVQLQQSGAVLARPGASVKMSCKGSGYTFTNYWMHWVKQRPGQGLEWIGATYRGHSDTYYNQKFKGKAKLTAVTSTSTAYMELSSLTNEDSAVYYCTRGAIYNGYDVLDNWGQGTLVTVSS

SEQ ID NO: 8 - DNA, 363 bp, Mus musculus
gaggtgcagctgcagcagagcggcgccgtgctggccaggcccggagctagcgtgaagatgagctgcaagggcagcggctacaccttcaccaactactggatgcactgggtgaaacagaggcccggccagggactggagtggatcggcgccacctacaggggccacagcgacacctactacaaccagaagttcaagggcaaggccaagctgaccgccgtgacctcaaccagcaccgcctacatggaactgagcagcctgaccaacgaggacagcgccgtctattactgcaccaggggcgccatctacaacggctacgacgtgctggacaattggggccagggaacactagtgaccgtgtccagc

SEQ ID NO: 9 - protein, 108 aa, Mus musculus
DIQLTQTTSSLSASLGDRVTISCSASQDISNYLNWYQQKPDGTVELVIYYTSNLHSGVPSRFSGSGSGTDYSLTIGYLEPEDVATYYCQQYRKLPWTFGGGSKLEIKR

SEQ ID NO: 10 - DNA, 324 bp, Mus musculus
gatatccagctgacccagaccacaagcagcctgagcgcctccctgggcgacagggtgaccattagctgcagcgccagccaggacatcagcaactacctgaactggtaccagcagaagcccgacggcaccgtggagctcgtgatctactacacctccaacctgcacagcggcgtgcccagcaggttctctggcagcggcagcggcaccgactacagcctgaccatcggctatctggagcccgaggacgtcgccacctactactgccagcagtacaggaagctgccctggaccttcggcggaggctctaagctggagattaagcgt

SEQ ID NO: 11 - protein, 121 aa, Artificial Sequence
  source: /note=“Description of Artificial Sequence Synthetic humanized antibody sequence”
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 12 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacaacggctacgacgtgctggacaactggggccagggcacactagtgaccgtgtccagc

SEQ ID NO: 13 - protein, 121 aa, Artificial Sequence
  source: /note=“Description of Artificial Sequence Synthetic humanized antibody sequence”
QVQLVQSGAEVKKPGSSVKVSCKASGYTFTNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 14 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggctacaccttcaccaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacaacggctacgacgtgctggacaactggggccagggcacactagtgaccgtgtccagc

SEQ ID NO: 15 - protein, 121 aa, Artificial Sequence
  source: /note=“Description of Artificial Sequence Synthetic humanized antibody sequence”
QVQLVQSGAEVKKPGSSVKVSCKASGYTFTNYWMHWVRQAPGQGLEWMGATYRGHSDTYYNQKFKGRVTITADKSTSTAYMELSSLRSEDTAVYYCTRGAIYNGYDVLDNWGQGTLVTVSS

SEQ ID NO: 16 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggctacaccttcaccaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcaccaggggcgccatctacaacggctacgacgtgctggacaactggggccagggcacactagtgaccgtgtccagc

SEQ ID NO: 17 - protein, 121 aa, Artificial Sequence
  source: /note=“Description of Artificial Sequence Synthetic humanized antibody sequence”
QVQLVQSGAEVKKPGSSVKVSCKGSGYTFTNYWMHWVRQAPGQGLEWMGATYRGHSDTYYNQKFKGRVTITADTSTSTAYMELSSLRSEDTAVYYCTRGAIYNGYDVLDNWGQGTLVTVSS

SEQ ID NO: 18 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaagggcagcggctacaccttcaccaactactggatgcactgggtgaggcaggcccccggacagggcctggagtggatgggcgccacctacaggggccacagcgacacctactacaaccagaagttcaagggccgggtgaccatcaccgccgacacgagcaccagcaccgcctacatggaactgagcagcctcaggagcgaggacaccgctgtgtattactgcaccaggggcgccatctacaacggctacgacgtgctggacaactggggccagggcacactagtgaccgtgtccagc

SEQ ID NO: 19 - protein, 121 aa, Artificial Sequence
  source: /note=“Description of Artificial Sequence Synthetic humanized antibody sequence”
QVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYNGYDVLDNWGQGTLVTVSS

SEQ ID NO: 20 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaagggcagcggctacaccttcaccaactactggatgcactgggtgaggcaggcccccggacagggcctggagtggatcggcgccacctacaggggccacagcgacacctactacaaccagaagttcaagggccgggcgaccctcaccgccgacacgagcaccagcaccgcctacatggaactgagcagcctcaggagcgaggacaccgctgtgtattactgcaccaggggcgccatctacaacggctacgacgtgctggacaactggggccagggcacactagtgaccgtgtccagc

SEQ ID NO: 21 - protein, 121 aa, Artificial Sequence
  source: /note=“Description of Artificial Sequence Synthetic humanized antibody sequence”
QVQLVQSGAEVKKPGSSVKVSCKGSGYTFTNYWMHWVRQAPGQGLEWMGATYRGHSDTYYNQKFKGRVTITADTSTSTAYMELSSLRSEDTAVYYCTRGAIYDGYDVLDNWGQGTLVTVSS

SEQ ID NO: 22 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaagggcagcggctacaccttcaccaactactggatgcactgggtgaggcaggcccccggacagggcctggagtggatgggcgccacctacaggggccacagcgacacctactacaaccagaagttcaagggccgggtgaccatcaccgccgacacgagcaccagcaccgcctacatggaactgagcagcctcaggagcgaggacaccgctgtgtattactgcaccaggggcgccatctacgacggctacgacgtgctggacaactggggccagggcacactagtgaccgtgtccagc

SEQ ID NO: 23 - protein, 121 aa, Artificial Sequence
  source: /note=“Description of Artificial Sequence Synthetic humanized antibody sequence”
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 24 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacgacggctacgacgtgctggacaactggggccagggcacactagtgaccgtgtccagc

SEQ ID NO: 25 - protein, 121 aa, Artificial Sequence
  source: /note=“Description of Artificial Sequence Synthetic humanized antibody sequence”
QVQLVQSGAEVKKPGSSVKVSCKASGYTFTNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 26 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggctacaccttcaccaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacgacggctacgacgtgctggacaactggggccagggcacactagtgaccgtgtccagc

SEQ ID NO: 27 - protein, 121 aa, Artificial Sequence
  source: /note=“Description of Artificial Sequence Synthetic humanized antibody sequence”
QVQLVQSGAEVKKPGSSVKVSCKASGYTFTNYWMHWVRQAPGQGLEWMGATYRGHSDTYYNQKFKGRVTITADKSTSTAYMELSSLRSEDTAVYYCTRGAIYDGYDVLDNWGQGTLVTVSS

SEQ ID NO: 28 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggctacaccttcaccaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcaccaggggcgccatctacgacggctacgacgtgctggacaactggggccagggcacactagtgaccgtgtccagc

SEQ ID NO: 29 - protein, 121 aa, Artificial Sequence
  source: /note=“Description of Artificial Sequence Synthetic humanized antibody sequence”
QVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSS

SEQ ID NO: 30 - DNA, 363 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaagggcagcggctacaccttcaccaactactggatgcactgggtgaggcaggcccccggacagggcctggagtggatcggcgccacctacaggggccacagcgacacctactacaaccagaagttcaagggccgggcgaccctcaccgccgacacgagcaccagcaccgcctacatggaactgagcagcctcaggagcgaggacaccgctgtgtattactgcaccaggggcgccatctacgacggctacgacgtgctggacaactggggccagggcacactagtgaccgtgtccagc

SEQ ID NO: 31 - protein, 108 aa, Artificial Sequence
  source: /note=“Description of Artificial Sequence Synthetic humanized antibody sequence”
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 32 - DNA, 324 bp, Artificial Sequence
  source: /note=“Description of Artificial Sequence Synthetic humanized antibody sequence”
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcccctggactttcggccagggcaccaaactggagatcaagcgt

SEQ ID NO: 33 - protein, 108 aa, Artificial Sequence
  source: /note=“Description of Artificial Sequence Synthetic humanized antibody sequence”
DIQMTQSPSSLSASVGDRVTITCSASQDISNYLNWYQQKPGKAPKLLIYYTSNLHSGVPSRFSGSGSGTDYTLTISSLQPEDFATYYCQQYRKLPWTFGQGTKLEIKR

SEQ ID NO: 34 - DNA, 324 bp, Artificial Sequence
  source: /note=“Description of Artificial Sequence Synthetic humanized antibody sequence”
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgattacaccctgaccatctccagcctgcagcccgaggacttcgccacctactactgccagcagtacaggaagctcccctggactttcggccagggcaccaaactggagatcaagcgt

SEQ ID NO: 35 - protein, 108 aa, Artificial Sequence
  source: /note=“Description of Artificial Sequence Synthetic humanized antibody sequence”
DIQLTQSPSSLSASVGDRVTITCSASQDISNYLNWYQQKPGKAPELVIYYTSNLHSGVPSRFSGSGSGTDYTLTISSLQPEDFATYYCQQYRKLPWTFGQGTKLEIKR

SEQ ID NO: 36 - DNA, 324 bp, Artificial Sequence
  source: /note=“Description of Artificial Sequence Synthetic humanized antibody sequence”
gacatccagctgacccagagccctagctcactgagcgccagcgtgggcgacagggtgaccattacctgctccgccagccaggacatcagcaactacctgaactggtaccagcagaagcccggcaaggcccccgagctggtgatctactacacctccaacctgcactccggcgtgcccagcaggttcagcggaagcggcagcggcaccgattacaccctgaccatctccagcctgcagcccgaggacttcgccacctactactgccagcagtacaggaagctcccctggactttcggccagggcaccaaactggagatcaagcgt

SEQ ID NO: 37 - protein, 310 aa, Homo sapiens
MPLLLLLPLLWAGALAMLQMAGQCSQNEYFDSLLHACIPCQLRCSSNTPPLTCQRYCNASVTNSVKGTNSGENLYFQGDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 930 bp, Homo sapiens
atgccgctgctgctactgctgcccctgctgtgggcaggggcgctagctatgctgcagatggccggccagtgcagccagaacgagtacttcgacagcctgctgcacgcctgcatcccctgccagctgagatgcagcagcaacacacctcctctgacctgccagagatactgcaacgccagcgtgaccaacagcgtgaagggcaccaactccggagagaacctgtacttccaaggggatcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 39 - protein, 307 aa, Homo sapiens
MPLLLLLPLLWAGALAMAGQCSQNEYFDSLLHACIPCQLRCSSNTPPLTCQRYCNASVTNSVKGTNSGENLYFQGDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 921 bp, Homo sapiens
atgccgctgctgctactgctgcccctgctgtgggcaggggcgctagctatggccggccagtgcagccagaacgagtacttcgacagcctgctgcacgcctgcatcccctgccagctgagatgcagcagcaacacacctcctctgacctgccagagatactgcaacgccagcgtgaccaacagcgtgaagggcaccaactccggagagaacctgtacttccaaggggatcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 41 - protein, 306 aa, Macaca fascicularis
MPLLLLLPLLWAGALAMARQCSQNEYFDSLLHDCKPCQLRCSSTPPLTCQRYCNASMTNSVKGMNSGENLYFQGDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 918 bp, Macaca fascicularis
atgccgctgctgctactgctgcccctgctgtgggcaggggcgctagctatggccagacagtgcagccagaacgagtacttcgacagcctgctgcacgactgcaagccctgccagctgagatgcagcagcacacctcctctgacctgccagagatactgcaacgccagcatgaccaacagcgtgaagggcatgaactccggagagaacctgtacttccaaggggatcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 43 - protein, 451 aa, Artificial Sequence
  source: /note=“Description of Artificial Sequence Synthetic humanized antibody sequence”
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacaacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 45 - protein, 451 aa, Artificial Sequence
  source: /note=“Description of Artificial Sequence Synthetic humanized antibody sequence”
QVQLVQSGAEVKKPGSSVKVSCKASGYTFTNYWMHWVRQAPGQGLEWMGATYRGHSDTYYNQKFKGRVTITADKSTSTAYMELSSLRSEDTAVYYCARGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggctacaccttcaccaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacaacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 47 - protein, 451 aa, Artificial Sequence
  source: /note=“Description of Artificial Sequence Synthetic humanized antibody sequence”
QVQLVQSGAEVKKPGSSVKVSCKASGYTFTNYWMHWVRQAPGQGLEWMGATYRGHSDTYYNQKFKGRVTITADKSTSTAYMELSSLRSEDTAVYYCTRGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggctacaccttcaccaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcaccaggggcgccatctacaacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 49 - protein, 451 aa, Artificial Sequence
  source: /note=“Description of Artificial Sequence Synthetic humanized antibody sequence”
QVQLVQSGAEVKKPGSSVKVSCKGSGYTFTNYWMHWVRQAPGQGLEWMGATYRGHSDTYYNQKFKGRVTITADTSTSTAYMELSSLRSEDTAVYYCTRGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaagggcagcggctacaccttcaccaactactggatgcactgggtgaggcaggcccccggacagggcctggagtggatgggcgccacctacaggggccacagcgacacctactacaaccagaagttcaagggccgggtgaccatcaccgccgacacgagcaccagcaccgcctacatggaactgagcagcctcaggagcgaggacaccgctgtgtattactgcaccaggggcgccatctacaacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 51 - protein, 451 aa, Artificial Sequence
  source: /note=“Description of Artificial Sequence Synthetic humanized antibody sequence”
QVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaagggcagcggctacaccttcaccaactactggatgcactgggtgaggcaggcccccggacagggcctggagtggatcggcgccacctacaggggccacagcgacacctactacaaccagaagttcaagggccgggcgaccctcaccgccgacacgagcaccagcaccgcctacatggaactgagcagcctcaggagcgaggacaccgctgtgtattactgcaccaggggcgccatctacaacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 53 - protein, 451 aa, Artificial Sequence
  source: /note=“Description of Artificial Sequence Synthetic humanized antibody sequence”
QVQLVQSGAEVKKPGSSVKVSCKGSGYTFTNYWMHWVRQAPGQGLEWMGATYRGHSDTYYNQKFKGRVTITADTSTSTAYMELSSLRSEDTAVYYCTRGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaagggcagcggctacaccttcaccaactactggatgcactgggtgaggcaggcccccggacagggcctggagtggatgggcgccacctacaggggccacagcgacacctactacaaccagaagttcaagggccgggtgaccatcaccgccgacacgagcaccagcaccgcctacatggaactgagcagcctcaggagcgaggacaccgctgtgtattactgcaccaggggcgccatctacgacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 55 - protein, 451 aa, Artificial Sequence
  source: /note=“Description of Artificial Sequence Synthetic humanized antibody sequence”
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacgacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 57 - protein, 451 aa, Artificial Sequence
  source: /note=“Description of Artificial Sequence Synthetic humanized antibody sequence”
QVQLVQSGAEVKKPGSSVKVSCKASGYTFTNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggctacaccttcaccaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacgacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 59 - protein, 451 aa, Artificial Sequence
  source: /note=“Description of Artificial Sequence Synthetic humanized antibody sequence”
QVQLVQSGAEVKKPGSSVKVSCKASGYTFTNYWMHWVRQAPGQGLEWMGATYRGHSDTYYNQKFKGRVTITADKSTSTAYMELSSLRSEDTAVYYCTRGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaaggccagcggctacaccttcaccaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcaccaggggcgccatctacgacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 61 - protein, 451 aa, Artificial Sequence
  source: /note=“Description of Artificial Sequence Synthetic humanized antibody sequence”
QVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - DNA, 1353 bp, Artificial Sequence
  source: /note=“Description of Artificial Sequence Synthetic humanized antibody sequence”
caggtgcagctggtccagagcggcgccgaagtgaagaagcccggcagctccgtgaaagtgagctgcaagggcagcggctacaccttcaccaactactggatgcactgggtgaggcaggcccccggacagggcctggagtggatcggcgccacctacaggggccacagcgacacctactacaaccagaagttcaagggccgggcgaccctcaccgccgacacgagcaccagcaccgcctacatggaactgagcagcctcaggagcgaggacaccgctgtgtattactgcaccaggggcgccatctacgacggctacgacgtgctggacaactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 63 - protein, 214 aa, Artificial Sequence
  source: /note=“Description of Artificial Sequence Synthetic humanized antibody sequence”
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - DNA, 642 bp, Artificial Sequence
  source: /note=“Description of Artificial Sequence Synthetic humanized antibody sequence”
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcccctggactttcggccagggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 65 - protein, 214 aa, Artificial Sequence
  source: /note=“Description of Artificial Sequence Synthetic humanized antibody sequence”
DIQMTQSPSSLSASVGDRVTITCSASQDISNYLNWYQQKPGKAPKLLIYYTSNLHSGVPSRFSGSGSGTDYTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - DNA, 642 bp, Artificial Sequence
  source: /note=“Description of Artificial Sequence Synthetic humanized antibody sequence”
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgattacaccctgaccatctccagcctgcagcccgaggacttcgccacctactactgccagcagtacaggaagctcccctggactttcggccagggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 67 - protein, 214 aa, Artificial Sequence
  source: /note=“Description of Artificial Sequence Synthetic humanized antibody sequence”
DIQLTQSPSSLSASVGDRVTITCSASQDISNYLNWYQQKPGKAPELVIYYTSNLHSGVPSRFSGSGSGTDYTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - DNA, 642 bp, Artificial Sequence
  source: /note=“Description of Artificial Sequence Synthetic humanized antibody sequence”
gacatccagctgacccagagccctagctcactgagcgccagcgtgggcgacagggtgaccattacctgctccgccagccaggacatcagcaactacctgaactggtaccagcagaagcccggcaaggcccccgagctggtgatctactacacctccaacctgcactccggcgtgcccagcaggttcagcggaagcggcagcggcaccgattacaccctgaccatctccagcctgcagcccgaggacttcgccacctactactgccagcagtacaggaagctcccctggactttcggccagggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 69 - protein, 115 aa, Mus musculus
EVQLQQSGPELVKPGASVKISCKASGYTFTDYYMKWVKQSHGKSLEWIGEIYPNNGGITYNQKFKGKATLTVDKSSSTAYMELRSLTSEDSAVYYCANGYEFVYWGQGTLVTVSA

SEQ ID NO: 70 - DNA, 345 bp, Mus musculus
gaggtccagttgcaacaatctggacctgagctggtgaagcctggggcttcagtgaagatatcctgtaaggcttctggatacacattcactgactactacatgaagtgggtgaagcagagccatggaaagagccttgagtggattggagagatttatcctaataatggtggtattacctacaaccagaagttcaagggcaaggccacattgactgtagacaagtcctccagcacagcctacatggagctccgcagcctgacatctgaggactctgcagtctattactgtgcaaatggttacgagtttgtttactggggccaagggactctggtcactgtctctgca

SEQ ID NO: 71 - protein, 108 aa, Mus musculus
DIQMTQTASSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIKR

SEQ ID NO: 72 - DNA, 324 bp, Mus musculus
gatatccagatgacacagactgcatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccgtggacgttcggtggaggcaccaagctggaaatcaaacgg

SEQ ID NO: 73 - protein, 445 aa, Artificial Sequence
  source: /note=“Description of Artificial Sequence Synthetic chimeric antibody sequence”
EVQLQQSGPELVKPGASVKISCKASGYTFTDYYMKWVKQSHGKSLEWIGEIYPNNGGITYNQKFKGKATLTVDKSSSTAYMELRSLTSEDSAVYYCANGYEFVYWGQGTLVTVSAAKTTAPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - DNA, 1335 bp, Artificial Sequence
  source: /note=“Description of Artificial Sequence Synthetic chimeric antibody sequence”
gaggtccagttgcaacaatctggacctgagctggtgaagcctggggcttcagtgaagatatcctgtaaggcttctggatacacattcactgactactacatgaagtgggtgaagcagagccatggaaagagccttgagtggattggagagatttatcctaataatggtggtattacctacaaccagaagttcaagggcaaggccacattgactgtagacaagtcctccagcacagcctacatggagctccgcagcctgacatctgaggactctgcagtctattactgtgcaaatggttacgagtttgtttactggggccaagggactctggtcactgtctctgcagccaaaacaacagcccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 75 - protein, 214 aa, Artificial Sequence
  source: /note=“Description of Artificial Sequence Synthetic chimeric antibody sequence”
DIQMTQTASSLSASLGDRVTISCSASQGISNYLNWYQQKPDGTVKLLIYYTSSLHSGVPSRFSGSGSGTDYSLTISNLEPEDIATYYCQQYSKLPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - DNA, 642 bp, Artificial Sequence
  source: /note=“Description of Artificial Sequence Synthetic chimeric antibody sequence”
gatatccagatgacacagactgcatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccgtggacgttcggtggaggcaccaagctggagctgaaacgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 77 - protein, 115 aa, Artificial Sequence
  source: /note=“Description of Artificial Sequence Synthetic humanized antibody sequence”
QVQLVQSGAEVKKPGSSVKVSCKASGGTFSDYYMKWVRQAPGQGLEWMGEIYPNNGGITYNQKFKGRVTITADKSTSTAYMELSSLRSEDTAVYYCARGYEFVYWGQGTLVTVSS

SEQ ID NO: 78 - DNA, 345 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggcggcaccttcagcgactactacatgaagtgggtgaggcaggcccccggccagggactggagtggatgggcgagatctaccccaacaacgggggcatcacctacaaccagaagttcaagggcagggtgaccatcaccgccgacaaaagcaccagcaccgcctacatggaactgagcagcctgaggagcgaggacaccgccgtgtactactgcgccaggggctacgagttcgtgtattggggccagggcacactagtgaccgtgtccagc

SEQ ID NO: 79 - protein, 115 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWMGEIYPNNGGITYNQKFKGRVTITADKSTSTAYMELSSLRSEDTAVYYCARGYEFVYWGQGTLVTVSS

SEQ ID NO: 80 - DNA, 345 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggatgggcgagatctaccccaacaacgggggcatcacctacaaccagaagttcaagggcagggtgaccatcaccgccgacaaaagcaccagcaccgcctacatggaactgagcagcctgaggagcgaggacaccgccgtgtactactgcgccaggggctacgagttcgtgtattggggccagggcacactagtgaccgtgtccagc

SEQ ID NO: 81 - protein, 115 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWMGEIYPNNGGITYNQKFKGRVTITADKSTSTAYMELSSLRSEDTAVYYCANGYEFVYWGQGTLVTVSS

SEQ ID NO: 82 - DNA, 345 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggatgggcgagatctaccccaacaacgggggcatcacctacaaccagaagttcaagggcagggtgaccatcaccgccgacaaaagcaccagcaccgcctacatggaactgagcagcctgaggagcgaggacaccgccgtgtactactgcgccaacggctacgagttcgtgtattggggccagggcacactagtgaccgtgtccagc

SEQ ID NO: 83 - protein, 115 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWIGEIYPNNGGITYNQKFKGRATLTVDKSTSTAYMELSSLRSEDTAVYYCANGYEFVYWGQGTLVTVSS

SEQ ID NO: 84 - DNA, 345 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggataggcgagatctaccccaacaacgggggcatcacctacaaccagaagttcaagggcagggcgaccctcaccgtcgacaaaagcaccagcaccgcctacatggaactgagcagcctgaggagcgaggacaccgccgtgtactactgcgccaacggctacgagttcgtgtattggggccagggcacactagtgaccgtgtccagc

SEQ ID NO: 85 - protein, 115 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWMGEIYPNNGGITYNQKFKGRVTITADKSTSTAYMELSSLRSEDTAVYYCADGYEFVYWGQGTLVTVSS

SEQ ID NO: 86 - DNA, 345 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggatgggcgagatctaccccaacaacgggggcatcacctacaaccagaagttcaagggcagggtgaccatcaccgccgacaaaagcaccagcaccgcctacatggaactgagcagcctgaggagcgaggacaccgccgtgtactactgcgccgacggctacgagttcgtgtattggggccagggcacactagtgaccgtgtccagc

SEQ ID NO: 87 - protein, 115 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWIGEIYPNNGGITYNQKFKGRATLTVDKSTSTAYMELSSLRSEDTAVYYCANGYEFDYWGQGTLVTVSS

SEQ ID NO: 88 - DNA, 345 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggataggcgagatctaccccaacaacgggggcatcacctacaaccagaagttcaagggcagggcgaccctcaccgtcgacaaaagcaccagcaccgcctacatggaactgagcagcctgaggagcgaggacaccgccgtgtactactgcgccaacggctacgagttcgactattggggccagggcacactagtgaccgtgtccagc

SEQ ID NO: 89 - protein, 108 aa, Artificial Sequence
  source: /note=“Description of Artificial Sequence Synthetic humanized antibody sequence”
DIQMTQSPSSLSASVGDRVTITCSASQGISNYLNWYQQKPGKAPKLLIYYTSSLHSGVPSRFSGSGSGTDFTLTISSLQPEDFATYYCQQYSKLPWTFGQGTKLEIKR

SEQ ID NO: 90 - DNA, 324 bp, Artificial Sequence
  source: /note=“Description of Artificial Sequence Synthetic humanized antibody sequence”
gacatccagatgacccagagcccctcaagcctgagcgccagcgtgggcgacagggtgactatcacctgcagcgcctcccagggcatcagcaactacctgaactggtaccagcagaagcccggcaaggcccctaagctgctgatctactacaccagcagcctgcacagcggcgtgcccagcaggttctccggcagcggcagcggaaccgacttcaccctgaccattagcagcctccagcccgaggacttcgccacctactactgccagcagtacagcaagctgccctggaccttcggccagggcaccaaactggagatcaagcgt

SEQ ID NO: 91 - protein, 108 aa, Artificial Sequence
  source: /note=“Description of Artificial Sequence Synthetic humanized antibody sequence”
DIQMTQSPSSLSASVGDRVTITCSASQGISNYLNWYQQKPGKAPKLLIYYTSSLHSGVPSRFSGSGSGTDYTLTISSLQPEDFATYYCQQYSKLPWTFGQGTKLEIKR

SEQ ID NO: 92 - DNA, 324 bp, Artificial Sequence
  source: /note=“Description of Artificial Sequence Synthetic humanized antibody sequence”
gacatccagatgacccagagcccctcaagcctgagcgccagcgtgggcgacagggtgactatcacctgcagcgcctcccagggcatcagcaactacctgaactggtaccagcagaagcccggcaaggcccctaagctgctgatctactacaccagcagcctgcacagcggcgtgcccagcaggttctccggcagcggcagcggaaccgactacaccctgaccattagcagcctccagcccgaggacttcgccacctactactgccagcagtacagcaagctgccctggaccttcggccagggcaccaaactggagatcaagcgt

SEQ ID NO: 93 - protein, 5 aa, Mus musculus
DYYMK

SEQ ID NO: 94 - protein, 17 aa, Mus musculus
EIYPNNGGITYNQKFKG

SEQ ID NO: 95 - protein, 6 aa, Mus musculus
GYEFVY

SEQ ID NO: 96 - protein, 11 aa, Mus musculus
SASQGISNYLN

SEQ ID NO: 97 - protein, 7 aa, Mus musculus
YTSSLHS

SEQ ID NO: 98 - protein, 9 aa, Mus musculus
QQYSKLPWT

SEQ ID NO: 99 - protein, 6 aa, Artificial Sequence
  source: /note=“Description of Artificial Sequence Synthetic humanized antibody sequence”
GYEFDY

SEQ ID NO: 100 - protein, 445 aa, Artificial Sequence
  source: /note=“Description of Artificial Sequence Synthetic humanized antibody sequence”
QVQLVQSGAEVKKPGSSVKVSCKASGGTFSDYYMKWVRQAPGQGLEWMGEIYPNNGGITYNQKFKGRVTITADKSTSTAYMELSSLRSEDTAVYYCARGYEFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - DNA, 1335 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggcggcaccttcagcgactactacatgaagtgggtgaggcaggcccccggccagggactggagtggatgggcgagatctaccccaacaacgggggcatcacctacaaccagaagttcaagggcagggtgaccatcaccgccgacaaaagcaccagcaccgcctacatggaactgagcagcctgaggagcgaggacaccgccgtgtactactgcgccaggggctacgagttcgtgtattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 102 - protein, 445 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWMGEIYPNNGGITYNQKFKGRVTITADKSTSTAYMELSSLRSEDTAVYYCARGYEFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - DNA, 1335 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggatgggcgagatctaccccaacaacgggggcatcacctacaaccagaagttcaagggcagggtgaccatcaccgccgacaaaagcaccagcaccgcctacatggaactgagcagcctgaggagcgaggacaccgccgtgtactactgcgccaggggctacgagttcgtgtattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 104 - protein, 445 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWMGEIYPNNGGITYNQKFKGRVTITADKSTSTAYMELSSLRSEDTAVYYCANGYEFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - DNA, 1335 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggatgggcgagatctaccccaacaacgggggcatcacctacaaccagaagttcaagggcagggtgaccatcaccgccgacaaaagcaccagcaccgcctacatggaactgagcagcctgaggagcgaggacaccgccgtgtactactgcgccaacggctacgagttcgtgtattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 106 - protein, 445 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWIGEIYPNNGGITYNQKFKGRATLTVDKSTSTAYMELSSLRSEDTAVYYCANGYEFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - DNA, 1335 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggataggcgagatctaccccaacaacgggggcatcacctacaaccagaagttcaagggcagggcgaccctcaccgtcgacaaaagcaccagcaccgcctacatggaactgagcagcctgaggagcgaggacaccgccgtgtactactgcgccaacggctacgagttcgtgtattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 108 - protein, 445 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWMGEIYPNNGGITYNQKFKGRVTITADKSTSTAYMELSSLRSEDTAVYYCADGYEFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - DNA, 1335 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggatgggcgagatctaccccaacaacgggggcatcacctacaaccagaagttcaagggcagggtgaccatcaccgccgacaaaagcaccagcaccgcctacatggaactgagcagcctgaggagcgaggacaccgccgtgtactactgcgccgacggctacgagttcgtgtattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 110 - protein, 445 aa, Artificial Sequence
  source: /note=“Description of Artificial Sequence Synthetic humanized antibody sequence”
QVQLVQSGAEVKKPGSSVKVSCKASGYTFTDYYMKWVRQAPGQGLEWIGEIYPNNGGITYNQKFKGRATLTVDKSTSTAYMELSSLRSEDTAVYYCANGYEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - DNA, 1335 bp, Artificial Sequence
  source: /note=“Description of Artificial Sequence Synthetic humanized antibody sequence”
caggtgcagctggtgcagagcggcgccgaagtgaagaagcccggctccagcgtgaaggtgagctgcaaggctagcggctacaccttcaccgactactacatgaagtgggtgaggcaggcccccggccagggactggagtggataggcgagatctaccccaacaacgggggcatcacctacaaccagaagttcaagggcagggcgaccctcaccgtcgacaaaagcaccagcaccgcctacatggaactgagcagcctgaggagcgaggacaccgccgtgtactactgcgccaacggctacgagttcgactattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 112 - protein, 214 aa, Artificial Sequence
  source: /note=“Description of Artificial Sequence Synthetic humanized antibody sequence”
DIQMTQSPSSLSASVGDRVTITCSASQGISNYLNWYQQKPGKAPKLLIYYTSSLHSGVPSRFSGSGSGTDFTLTISSLQPEDFATYYCQQYSKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - DNA, 642 bp, Artificial Sequence
  source: /note=“Description of Artificial Sequence Synthetic humanized antibody sequence”
gacatccagatgacccagagcccctcaagcctgagcgccagcgtgggcgacagggtgactatcacctgcagcgcctcccagggcatcagcaactacctgaactggtaccagcagaagcccggcaaggcccctaagctgctgatctactacaccagcagcctgcacagcggcgtgcccagcaggttctccggcagcggcagcggaaccgacttcaccctgaccattagcagcctccagcccgaggacttcgccacctactactgccagcagtacagcaagctgccctggaccttcggccagggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 114 - protein, 214 aa, Artificial Sequence
  source: /note=“Description of Artificial Sequence Synthetic humanized antibody sequence”
DIQMTQSPSSLSASVGDRVTITCSASQGISNYLNWYQQKPGKAPKLLIYYTSSLHSGVPSRFSGSGSGTDYTLTISSLQPEDFATYYCQQYSKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - DNA, 642 bp, Artificial Sequence
  source: /note=“Description of Artificial Sequence Synthetic humanized antibody sequence”
gacatccagatgacccagagcccctcaagcctgagcgccagcgtgggcgacagggtgactatcacctgcagcgcctcccagggcatcagcaactacctgaactggtaccagcagaagcccggcaaggcccctaagctgctgatctactacaccagcagcctgcacagcggcgtgcccagcaggttctccggcagcggcagcggaaccgactacaccctgaccattagcagcctccagcccgaggacttcgccacctactactgccagcagtacagcaagctgccctggaccttcggccagggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 116 - protein, 118 aa, Mus musculus
EVQLQQSGPVLVKPGASVKMSCEASGYTFTDYYMNWVKQSHGKTLEWIGVINPYNGGTDYNQKFKGKATLTVDKSSSTAYMELNSLTSEDSAVYYCARSVYDYPFDYWGQGTLVTVSS

SEQ ID NO: 117 - DNA, 354 bp, Mus musculus
gaggtgcagctgcagcagagcggccccgtgctggtgaagcctggagccagcgtgaaaatgagctgcgaagccagcggctacaccttcaccgactactacatgaactgggtgaagcagagccacggcaagaccctggagtggatcggcgtgatcaacccctacaacgggggcaccgactacaaccagaagttcaagggcaaggccactctgaccgtggacaagagctccagcaccgcctacatggaactgaacagcctcacctctgaggacagcgccgtctattactgcgccaggagcgtgtacgactaccccttcgactactggggccagggcacactagtgaccgtgtccagc

SEQ ID NO: 118 - protein, 448 aa, Artificial Sequence
  source: /note=“Description of Artificial Sequence Synthetic chimeric antibody sequence”
EVQLQQSGPVLVKPGASVKMSCEASGYTFTDYYMNWVKQSHGKTLEWIGVINPYNGGTDYNQKFKGKATLTVDKSSSTAYMELNSLTSEDSAVYYCARSVYDYPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - DNA, 1344 bp, Artificial Sequence
  source: /note=“Description of Artificial Sequence Synthetic chimeric antibody sequence”
gaggtgcagctgcagcagagcggccccgtgctggtgaagcctggagccagcgtgaaaatgagctgcgaagccagcggctacaccttcaccgactactacatgaactgggtgaagcagagccacggcaagaccctggagtggatcggcgtgatcaacccctacaacgggggcaccgactacaaccagaagttcaagggcaaggccactctgaccgtggacaagagctccagcaccgcctacatggaactgaacagcctcacctctgaggacagcgccgtctattactgcgccaggagcgtgtacgactaccccttcgactactggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 120 - protein, 111 aa, Mus musculus
DIVLTQSPASLAVSLGQRATISCRASESVSIHGTHLMHWYQQKPGQPPKLLIYAASNLESGVPARFSGSGSETDFTLNIHPVEEEDAATYFCQQSIEDPRTFGGGTKLEIK

SEQ ID NO: 121 - DNA, 336 bp, Mus musculus
gacatcgtcctgacccagagccccgccagcctggccgtgagcctgggccagagggccacaatcagctgcagggcctctgagtccgtgagcatccacggcacccacctgatgcactggtatcagcagaagcccggccagcctcccaagctgctgatctacgccgccagcaacctggagagcggcgtgcccgctaggttcagcggaagcggcagcgagaccgacttcaccctgaacatccaccccgtggaggaggaagacgccgccacctacttctgccagcagagcatcgaggaccccaggaccttcggcgggggcaccaagctcgagattaagcgt

SEQ ID NO: 122 - protein, 237 aa, Artificial Sequence
  source: /note=“Description of Artificial Sequence Synthetic chimeric antibody sequence”
MGWSCIILFLVATATGVHSDIVLTQSPASLAVSLGQRATISCRASESVSIHGTHLMHWYQQKPGQPPKLLIYAASNLESGVPARFSGSGSETDFTLNIHPVEEEDAATYFCQQSIEDPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 711 bp, Artificial Sequence
  source: /note=“Description of Artificial Sequence Synthetic chimeric antibody sequence”
atgggctggtcctgcatcatcctgtttctggtggccaccgccaccggcgtgcacagcgacatcgtcctgacccagagccccgccagcctggccgtgagcctgggccagagggccacaatcagctgcagggcctctgagtccgtgagcatccacggcacccacctgatgcactggtatcagcagaagcccggccagcctcccaagctgctgatctacgccgccagcaacctggagagcggcgtgcccgctaggttcagcggaagcggcagcgagaccgacttcaccctgaacatccaccccgtggaggaggaagacgccgccacctacttctgccagcagagcatcgaggaccccaggaccttcggcgggggcaccaagctcgagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 124 - protein, 119 aa, Mus musculus
EVQLQQSGPELVKPGTSVKIPCKTSGYIFTDYSIDWVKQSHGKSLEWIGDIDPNYGDPIYNHKFKGKATLTVDRSSSTAYMELRSLTSEDTAVYFCARRATGTDWFAFWGQGTLVTVSS

SEQ ID NO: 125 - DNA, 357 bp, Mus musculus
gaggtgcagctgcagcagagcggccccgagctggtgaaacccggcaccagcgtgaagatcccctgcaagacctctggctacatcttcaccgactacagcatcgactgggtgaagcagagccacggcaagtctctggagtggattggggacatcgaccccaactacggcgaccccatctacaaccacaagttcaagggcaaggccaccctgaccgtggacaggagcagcagcaccgcctacatggaactcaggagcctgaccagcgaggacaccgccgtgtatttttgcgccaggagggccaccggcactgattggttcgccttctggggccagggcacactagtgaccgtgtccagc

SEQ ID NO: 126 - protein, 449 aa, Artificial Sequence
  source: /note=“Description of Artificial Sequence Synthetic chimeric antibody sequence”
EVQLQQSGPELVKPGTSVKIPCKTSGYIFTDYSIDWVKQSHGKSLEWIGDIDPNYGDPIYNHKFKGKATLTVDRSSSTAYMELRSLTSEDTAVYFCARRATGTDWFAFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - DNA, 1347 bp, Artificial Sequence
  source: /note=“Description of Artificial Sequence Synthetic chimeric antibody sequence”
gaggtgcagctgcagcagagcggccccgagctggtgaaacccggcaccagcgtgaagatcccctgcaagacctctggctacatcttcaccgactacagcatcgactgggtgaagcagagccacggcaagtctctggagtggattggggacatcgaccccaactacggcgaccccatctacaaccacaagttcaagggcaaggccaccctgaccgtggacaggagcagcagcaccgcctacatggaactcaggagcctgaccagcgaggacaccgccgtgtatttttgcgccaggagggccaccggcactgattggttcgccttctggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 128 - protein, 108 aa, Mus musculus
DIQMTQSPASLSVSVGETVTITCRASENIYNNLAWYQQKQGKSPQLLVYAATILADGVPSRFSGSGSGTQYSLKINSLQSGDFGTYYCQHFWGTPLTFGAGTKLELKR

SEQ ID NO: 129 - DNA, 324 bp, Mus musculus
gacatccagatgacccagagccccgctagcctcagcgtgtccgtcggcgagaccgtgaccatcacctgcagggccagcgagaacatctacaacaacctggcctggtatcagcagaagcagggcaaaagcccccagctgctggtgtacgccgccaccattctggccgacggcgtgcccagcaggttctctggaagcggcagcggcacccagtacagcctgaagatcaacagcctgcagagcggggacttcggcacctactactgccagcacttctggggcactcccctgaccttcggagccggcaccaagctggagctgaagcgt

SEQ ID NO: 130 - protein, 214 aa, Artificial Sequence
  source: /note=“Description of Artificial Sequence Synthetic chimeric antibody sequence”
DIQMTQSPASLSVSVGETVTITCRASENIYNNLAWYQQKQGKSPQLLVYAATILADGVPSRFSGSGSGTQYSLKINSLQSGDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - DNA, 642 bp, Artificial Sequence
  source: /note=“Description of Artificial Sequence Synthetic chimeric antibody sequence”
gacatccagatgacccagagccccgctagcctcagcgtgtccgtcggcgagaccgtgaccatcacctgcagggccagcgagaacatctacaacaacctggcctggtatcagcagaagcagggcaaaagcccccagctgctggtgtacgccgccaccattctggccgacggcgtgcccagcaggttctctggaagcggcagcggcacccagtacagcctgaagatcaacagcctgcagagcggggacttcggcacctactactgccagcacttctggggcactcccctgaccttcggagccggcaccaagctggagctgaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 132 - protein, 118 aa, Mus musculus
QVQLQQPGAELVKPGASVKLSCKASGYTFTNYWMHWVKQRPGQGLEWIGIIHPNSGSTNYNEKFKSKATLTVDKSSSTAYMQLSSLTSEDSAVYYCARGIYDYPFAYWGQGTLVTVSS

SEQ ID NO: 133 - DNA, 354 bp, Mus musculus
caggtgcagctccagcagcccggagccgaactggtgaagcccggagccagcgtcaaactgtcctgcaaggccagcggctacaccttcaccaactactggatgcactgggtgaagcagaggcccggccagggcctggagtggatcggcatcatccaccccaacagcgggagcaccaactacaacgagaagttcaagagcaaggccaccctgaccgtggacaagagcagcagcactgcctacatgcagctgagcagcctgaccagcgaggacagcgctgtgtactactgcgccaggggcatctacgactaccccttcgcctattggggccagggcacactagtgaccgtgtccagc

SEQ ID NO: 134 - protein, 448 aa, Artificial Sequence
  source: /note=“Description of Artificial Sequence Synthetic chimeric antibody sequence”
QVQLQQPGAELVKPGASVKLSCKASGYTFTNYWMHWVKQRPGQGLEWIGIIHPNSGSTNYNEKFKSKATLTVDKSSSTAYMQLSSLTSEDSAVYYCARGIYDYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - DNA, 1344 bp, Artificial Sequence
  source: /note=“Description of Artificial Sequence Synthetic chimeric antibody sequence”
caggtgcagctccagcagcccggagccgaactggtgaagcccggagccagcgtcaaactgtcctgcaaggccagcggctacaccttcaccaactactggatgcactgggtgaagcagaggcccggccagggcctggagtggatcggcatcatccaccccaacagcgggagcaccaactacaacgagaagttcaagagcaaggccaccctgaccgtggacaagagcagcagcactgcctacatgcagctgagcagcctgaccagcgaggacagcgctgtgtactactgcgccaggggcatctacgactaccccttcgcctattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 136 - protein, 112 aa, Mus musculus
DIVLTQSPASLAVSLGQRATISCRASESVSIHGTHLMHWYQQKPGQPPKLLIYAASNLESGVPARFSGSGSETDFTLNIHPVEEEDAATYFCQQSIEDPYTFGGGTKLEIKR

SEQ ID NO: 137 - DNA, 336 bp, Mus musculus
gacatcgtgctgacccagtctcccgctagcctggccgtgtctctgggccagagggccacaatcagctgcagggccagcgagagcgtcagcattcacggcacccacctgatgcactggtaccagcagaagcccggccagcctcccaagctcctgatctacgccgccagcaacctggaaagcggagtgcccgccaggttcagcggcagcggctccgagaccgacttcaccctgaacatccaccccgtggaggaggaggacgccgccacctacttctgccagcagagcatcgaggacccctacaccttcggcggcggcaccaagctggagatcaagcgt

SEQ ID NO: 138 - protein, 218 aa, Artificial Sequence
  source: /note=“Description of Artificial Sequence Synthetic chimeric antibody sequence”
DIVLTQSPASLAVSLGQRATISCRASESVSIHGTHLMHWYQQKPGQPPKLLIYAASNLESGVPARFSGSGSETDFTLNIHPVEEEDAATYFCQQSIEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - DNA, 654 bp, Artificial Sequence
  source: /note=“Description of Artificial Sequence Synthetic chimeric antibody sequence”
gacatcgtgctgacccagtctcccgctagcctggccgtgtctctgggccagagggccacaatcagctgcagggccagcgagagcgtcagcattcacggcacccacctgatgcactggtaccagcagaagcccggccagcctcccaagctcctgatctacgccgccagcaacctggaaagcggagtgcccgccaggttcagcggcagcggctccgagaccgacttcaccctgaacatccaccccgtggaggaggaggacgccgccacctacttctgccagcagagcatcgaggacccctacaccttcggcggcggcaccaagctggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 140 - protein, 120 aa, Mus musculus
EVKLLQSGGGLVQPGGSLKLSCAASGIDFSRYWMSWVRRAPGKGLEWIGEINPDRSTINYAPSLKDKFIISRDNAKNTLYLQMSKVRSEDTALYYCAVFYYDYEGAMDYWGQGTSVTVSS

SEQ ID NO: 141 - DNA, 360 bp, Mus musculus
gaggtgaagcttctccagtctggaggtggcctggtgcagcctggaggatccctgaaactctcctgtgcagcctcaggaatcgattttagtagatactggatgagttgggttcggcgggctccagggaaaggactagaatggattggagaaattaatccagataggagtacaatcaactatgcaccatctctaaaggataaattcatcatctccagagacaacgccaaaaatacgctgtacctgcaaatgagcaaagtgagatctgaggacacagccctttattactgtgcagttttctactatgattacgagggtgctatggactactggggtcaaggaacctcagtcaccgtctcctca

SEQ ID NO: 142 - protein, 450 aa, Artificial Sequence
  source: /note=“Description of Artificial Sequence Synthetic chimeric antibody sequence”
EVKLLQSGGGLVQPGGSLKLSCAASGIDFSRYWMSWVRRAPGKGLEWIGEINPDRSTINYAPSLKDKFIISRDNAKNTLYLQMSKVRSEDTALYYCAVFYYDYEGAMDYWGQGTSVTVSSAKTTAPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - DNA, 1350 bp, Artificial Sequence
  source: /note=“Description of Artificial Sequence Synthetic chimeric antibody sequence”
gaggtgaagcttctccagtctggaggtggcctggtgcagcctggaggatccctgaaactctcctgtgcagcctcaggaatcgattttagtagatactggatgagttgggttcggcgggctccagggaaaggactagaatggattggagaaattaatccagataggagtacaatcaactatgcaccatctctaaaggataaattcatcatctccagagacaacgccaaaaatacgctgtacctgcaaatgagcaaagtgagatctgaggacacagccctttattactgtgcagttttctactatgattacgagggtgctatggactactggggtcaaggaacctcagtcaccgtctcctcagccaaaacaacagcccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 144 - protein, 108 aa, Mus musculus
DIVMTQSQKFMSTSVGDRVSVTCKASQNVDTNVAWYQQKPGQSPKALIYSASYRFSGVPDRFTGSGSGTDFTLTISNVQSEDLAEYFCQQYNSFPFTFGSGTKLEIKR

SEQ ID NO: 145 - DNA, 324 bp, Mus musculus
gacattgtgatgacccagtctcaaaaattcatgtccacatcagtaggagacagggtcagcgtcacctgcaaggccagtcagaatgtggatactaatgtagcctggtatcaacaaaaaccagggcaatctcctaaagcactgatttactcggcatcctaccggttcagtggagtccctgatcgcttcacaggcagtggatctgggacagatttcactctcaccatcagcaatgtgcagtctgaagacttggcagagtatttctgtcagcaatataacagctttccattcacgttcggctcggggacaaagttggaaataaaacgt

SEQ ID NO: 146 - protein, 214 aa, Artificial Sequence
  source: /note=“Description of Artificial Sequence Synthetic chimeric antibody sequence”
DIVMTQSQKFMSTSVGDRVSVTCKASQNVDTNVAWYQQKPGQSPKALIYSASYRFSGVPDRFTGSGSGTDFTLTISNVQSEDLAEYFCQQYNSFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - DNA, 642 bp, Artificial Sequence
  source: /note=“Description of Artificial Sequence Synthetic chimeric antibody sequence”
gacattgtgatgacccagtctcaaaaattcatgtccacatcagtaggagacagggtcagcgtcacctgcaaggccagtcagaatgtggatactaatgtagcctggtatcaacaaaaaccagggcaatctcctaaagcactgatttactcggcatcctaccggttcagtggagtccctgatcgcttcacaggcagtggatctgggacagatttcactctcaccatcagcaatgtgcagtctgaagacttggcagagtatttctgtcagcaatataacagctttccattcacgttcggctcggggacaaagttggaaataaaacgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 148 - protein, 118 aa, Mus musculus
PVQLQQPGTELVRPGTSVKLSCKASGYTFTSYWMHWVKQRPGQGLEWIGVIDPSDSYTNYNQKFKGKATLTVDTSSSTAYMQLSSLTSEDSAVYYCARQVFDYPMDYWGQGTSVTVSS

SEQ ID NO: 149 - DNA, 354 bp, Mus musculus
ccggtccaactgcagcagcctgggactgagctggtgaggcctgggacttcagtgaagttgtcctgcaaggcttctggctacaccttcaccagctactggatgcactgggtaaagcagaggcctggacaaggccttgagtggatcggagtgattgatccttctgatagttatactaactacaatcaaaagttcaagggcaaggccacattgactgtagacacatcctccagcacagcctacatgcagctcagcagcctgacatctgaggactctgcggtctattactgtgcaagacaggtgtttgactatcctatggactactggggtcaaggaacctcagtcaccgtctcctca

SEQ ID NO: 150 - protein, 448 aa, Artificial Sequence
  source: /note=“Description of Artificial Sequence Synthetic chimeric antibody sequence”
PVQLQQPGTELVRPGTSVKLSCKASGYTFTSHWMHWVKQRPGQGLEWIGVIDPSDSYTNYNQKFKGKATLTVDTSSSTAYMQLSSLTSEDSAVYYCARQVFDYPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - DNA, 1344 bp, Artificial Sequence
  source: /note=“Description of Artificial Sequence Synthetic chimeric antibody sequence”
ccggtccaactgcagcagcctgggactgagctggtgaggcctgggacttcagtgaagttgtcctgcaaggcttctggctacaccttcaccagccactggatgcactgggtaaagcagaggcctggacaaggccttgagtggatcggagtgattgatccttctgatagttatactaactacaatcaaaagttcaagggcaaggccacattgactgtagacacatcctccagcacagcctacatgcagctcagcagcctgacatctgaggactctgcggtctattactgtgcaagacaggtgtttgactatcctatggactactggggtcaaggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 152 - protein, 112 aa, Mus musculus
DIVLTQSPASLAVSLGQRATISCRASESVSIHGTHLMHWYQQKPGQPPKLLIYAASNLESGVPARFSGSGSETDFTLNIHPVEEEDAATYFCQQSIEDPWTFGGGTKLEIKR

SEQ ID NO: 153 - DNA, 336 bp, Mus musculus
gacattgtgctgacccaatctccagcttctttggctgtgtctctagggcagagggccaccatctcctgcagagccagtgaaagtgtcagtattcatggtactcatttaatgcactggtaccaacagaaaccaggacagccacccaaactcctcatctatgctgcatccaacctagaatctggagtccctgccaggttcagtggcagtgggtctgagacagacttcaccctcaacatccatcctgtggaggaggaggatgctgcaacctatttctgtcagcaaagtattgaggatccgtggacgttcggtggaggcaccaagctggaaatcaaacgt

SEQ ID NO: 154 - protein, 218 aa, Artificial Sequence
  source: /note=“Description of Artificial Sequence Synthetic chimeric antibody sequence”
DIVLTQSPASLAVSLGQRATISCRASESVSIHGTHLMHWYQQKPGQPPKLLIYAASNLESGVPARFSGSGSETDFTLNIHPVEEEDAATYFCQQSIEDPWTFGGGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - DNA, 654 bp, Artificial Sequence
  source: /note=“Description of Artificial Sequence Synthetic chimeric antibody sequence”
gacattgtgctgacccaatctccagcttctttggctgtgtctctagggcagagggccaccatctcctgcagagccagtgaaagtgtcagtattcatggtactcatttaatgcactggtaccaacagaaaccaggacagccacccaaactcctcatctatgctgcatccaacctagaatctggagtccctgccaggttcagtggcagtgggtctgagacagacttcaccctcaacatccatcctgtggaggaggaggatgctgcaacctatttctgtcagcaaagtattgaggatccgtggacgttcggtggaggcaccaagctggaaatcaatcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 156 - protein, 118 aa, Mus musculus
QVQLQQSGAELVRPGASVTLSCKASGYTFTDYEMHWVKQTPVHGLEWIGAIDPETGGTAYNQKFKGKAILTADKSSSTAYMELRSLTSEDSAVYYCTRSIYDYYFDYWGQGTTLTVSS

SEQ ID NO: 157 - DNA, 354 bp, Mus musculus
caggttcaactgcagcagtctggggctgagctggtgaggcctggggcttcagtgacgctgtcctgcaaggcttcgggctacacatttactgactatgaaatgcactgggtgaagcagacacctgtgcatggcctggaatggattggagctattgatcctgaaactggtggtactgcctacaatcagaagttcaagggcaaggccatactgactgcagacaaatcctccagcacagcctacatggagctccgcagcctgacatctgaggactctgccgtctattactgtacaagatcgatttatgattactactttgactactggggccaaggcaccactctcacagtctcctca

SEQ ID NO: 158 - protein, 448 aa, Artificial Sequence
  source: /note=“Description of Artificial Sequence Synthetic chimeric antibody sequence”
QVQLQQSGAELVRPGASVTLSCKASGYTFTDYEMHWVKQTPVHGLEWIGAIDPETGGTAYNQKFKGKAILTADKSSSTAYMELRSLTSEDSAVYYCTRSIYDYYFDYWGQGTTLTVSSAKTTPPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - DNA, 1344 bp, Artificial Sequence
  source: /note=“Description of Artificial Sequence Synthetic chimeric antibody sequence”
caggttcaactgcagcagtctggggctgagctggtgaggcctggggcttcagtgacgctgtcctgcaaggcttcgggctacacatttactgactatgaaatgcactgggtgaagcagacacctgtgcatggcctggaatggattggagctattgatcctgaaactggtggtactgcctacaatcagaagttcaagggcaaggccatactgactgcagacaaatcctccagcacagcctacatggagctccgcagcctgacatctgaggactctgccgtctattactgtacaagatcgatttatgattactactttgactactggggccaaggcaccactctcacagtctcctcagccaaaacgacaccccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 160 - protein, 112 aa, Mus musculus
DIVLTQSPASLAVSLGQRATISCRASESVSIHGTHLMHWYQQKPGQPPKLLIYAASNLESGVPARFSGGGSETDFTLNIHPVEEEDGATYFCQQSIEYPRTFGGGTKLEINR

SEQ ID NO: 161 - DNA, 336 bp, Mus musculus
gacattgtgctgacccaatctccagcttctttggctgtgtctctagggcagagggccaccatctcctgcagagccagtgaaagtgtcagtattcatggtactcatttaatgcactggtaccaacagaaaccaggacagccacccaaactcctcatctatgctgcatccaacctagaatctggagtccctgccaggttcagtggcggtgggtctgagacagacttcaccctcaacatccatcctgtggaggaggaggatggtgcaacctatttctgtcagcaaagtattgagtatcctcggacgttcggtggaggcaccaagctggaaatcaatcgt

SEQ ID NO: 162 - protein, 218 aa, Artificial Sequence
  source: /note=“Description of Artificial Sequence Synthetic chimeric antibody sequence”
DIVLTQSPASLAVSLGQRATISCRASESVSIHGTHLMHWYQQKPGQPPKLLIYAASNLESGVPARFSGGGSETDFTLNIHPVEEEDGATYFCQQSIEYPRTFGGGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - DNA, 654 bp, Artificial Sequence
  source: /note=“Description of Artificial Sequence Synthetic chimeric antibody sequence”
gacattgtgctgacccaatctccagcttctttggctgtgtctctagggcagagggccaccatctcctgcagagccagtgaaagtgtcagtattcatggtactcatttaatgcactggtaccaacagaaaccaggacagccacccaaactcctcatctatgctgcatccaacctagaatctggagtccctgccaggttcagtggcggtgggtctgagacagacttcaccctcaacatccatcctgtggaggaggaggatggtgcaacctatttctgtcagcaaagtattgagtatcctcggacgttcggtggaggcaccaagctggaaatcaatcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 164 - protein, 5 aa, Mus musculus
DYYNM

SEQ ID NO: 165 - protein, 16 aa, Mus musculus
VINPYNGGTDYNQKFG

SEQ ID NO: 166 - protein, 9 aa, Mus musculus
SVYDYPFDY

SEQ ID NO: 167 - protein, 15 aa, Mus musculus
RASESVSIHGTHLMH

SEQ ID NO: 168 - protein, 7 aa, Mus musculus
AASNLES

SEQ ID NO: 169 - protein, 9 aa, Mus musculus
QQSIEDPRT

SEQ ID NO: 170 - protein, 5 aa, Mus musculus
DYSID

SEQ ID NO: 171 - protein, 17 aa, Mus musculus
DIDPNYGDPIYNHKFKG

SEQ ID NO: 172 - protein, 10 aa, Mus musculus
RATGTDWFAF

SEQ ID NO: 173 - protein, 11 aa, Mus musculus
RASENIYNNLA

SEQ ID NO: 174 - protein, 7 aa, Mus musculus
AATILAD

SEQ ID NO: 175 - protein, 9 aa, Mus musculus
QHFWGTPLT

SEQ ID NO: 176 - protein, 5 aa, Mus musculus
NYWMH

SEQ ID NO: 177 - protein, 17 aa, Mus musculus
IIHPNSGSTNYNEKFKS

SEQ ID NO: 178 - protein, 9 aa, Mus musculus
GIYDYPFAY

SEQ ID NO: 179 - protein, 15 aa, Mus musculus
RASESVSIHGTHLMH

SEQ ID NO: 180 - protein, 7 aa, Mus musculus
AASNLES

SEQ ID NO: 181 - protein, 9 aa, Mus musculus
QQSIEDPYT

SEQ ID NO: 182 - protein, 5 aa, Mus musculus
RYWMS

SEQ ID NO: 183 - protein, 17 aa, Mus musculus
EINPDRSTINYAPSLKD

SEQ ID NO: 184 - protein, 11 aa, Mus musculus
FYYDYEGAMDY

SEQ ID NO: 185 - protein, 11 aa, Mus musculus
KASQNVDTNVA

SEQ ID NO: 186 - protein, 7 aa, Mus musculus
SASYRFS

SEQ ID NO: 187 - protein, 9 aa, Mus musculus
QQYNSFPFT

SEQ ID NO: 188 - protein, 5 aa, Mus musculus
SYWMH

SEQ ID NO: 189 - protein, 17 aa, Mus musculus
VIDPSDSYTNYNQKFKG

SEQ ID NO: 190 - protein, 9 aa, Mus musculus
QVFDYPMDY

SEQ ID NO: 191 - protein, 15 aa, Mus musculus
RASESVSIHGTHLMH

SEQ ID NO: 192 - protein, 7 aa, Mus musculus
AASNLES

SEQ ID NO: 193 - protein, 9 aa, Mus musculus
QQSIEDPWT

SEQ ID NO: 194 - protein, 5 aa, Mus musculus
DYEMH

SEQ ID NO: 195 - protein, 17 aa, Mus musculus
AIDPETGGTAYNQKFKG

SEQ ID NO: 196 - protein, 9 aa, Mus musculus
SIYDYYFDY

SEQ ID NO: 197 - protein, 15 aa, Mus musculus
RASESVSIHGTHLMH

SEQ ID NO: 198 - protein, 7 aa, Mus musculus
AASNLES

SEQ ID NO: 199 - protein, 9 aa, Mus musculus
QQSIEYPRT

SEQ ID NO: 200 - protein, 12 aa, Artificial Sequence
  source: /note=“Description of Artificial Sequence Synthetic humanized antibody sequence”
GAIYDGYDVLDN

SEQ ID NO: 201 - protein, 5 aa, Artificial Sequence
  source: /note=“Description of Artificial Sequence Synthetic peptide”
NYYMY

SEQ ID NO: 202 - protein, 17 aa, Artificial Sequence
  source: /note=“Description of Artificial Sequence Synthetic peptide”
GINPSNGGTNFNEKFKN

SEQ ID NO: 203 - protein, 11 aa, Artificial Sequence
  source: /note=“Description of Artificial Sequence Synthetic peptide”
RDYRFDMGFDY

SEQ ID NO: 204 - protein, 15 aa, Artificial Sequence
  source: /note=“Description of Artificial Sequence Synthetic peptide”
RASKGVSTSGYSYLH

SEQ ID NO: 205 - protein, 7 aa, Artificial Sequence
  source: /note=“Description of Artificial Sequence Synthetic peptide”
LASYLES

SEQ ID NO: 206 - protein, 9 aa, Artificial Sequence
  source: /note=“Description of Artificial Sequence Synthetic peptide”
QHSRDLPLT

SEQ ID NO: 207 - protein, 120 aa, Artificial Sequence [sequence omitted]

SEQ ID NO: 208 - protein, 111 aa, Artificial Sequence [sequence omitted]

SEQ ID NO: 209 - protein, 446 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 210 - protein, 218 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 5 aa, Artificial Sequence
  source: /note=“Description of Artificial Sequence Synthetic peptide”
NSGMH

SEQ ID NO: 212 - protein, 17 aa, Artificial Sequence
  source: /note=“Description of Artificial Sequence Synthetic peptide”
VIWYDGSKRYYADSVKG

SEQ ID NO: 213 - protein, 4 aa, Artificial Sequence
  source: /note=“Description of Artificial Sequence Synthetic peptide”
NDDY

SEQ ID NO: 214 - protein, 11 aa, Artificial Sequence
  source: /note=“Description of Artificial Sequence Synthetic peptide”
RASQSVSSYLA

SEQ ID NO: 215 - protein, 7 aa, Artificial Sequence
  source: /note=“Description of Artificial Sequence Synthetic peptide”
DASNRAT

SEQ ID NO: 216 - protein, 9 aa, Artificial Sequence
  source: /note=“Description of Artificial Sequence Synthetic peptide”
QQSSNWPRT

SEQ ID NO: 217 - protein, 113 aa, Artificial Sequence [sequence omitted]

SEQ ID NO: 218 - protein, 107 aa, Artificial Sequence [sequence omitted]

SEQ ID NO: 219 - protein, 5 aa, Mus musculus
DYSMH

SEQ ID NO: 220 - protein, 17 aa, Mus musculus
WINTETGEPTYADDFKG

SEQ ID NO: 221 - protein, 13 aa, Mus musculus
PYYDYVSYYAMDY

SEQ ID NO: 222 - protein, 11 aa, Mus musculus
KASQDVSTAVA

SEQ ID NO: 223 - protein, 7 aa, Mus musculus
SASYLYT

SEQ ID NO: 224 - protein, 9 aa, Mus musculus
QQHYSTPRT

SEQ ID NO: 225 - protein, 122 aa, Artificial Sequence
  source: /note=“Description of Artificial Sequence Synthetic polypeptide”
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 226 - DNA, 455 bp, Artificial Sequence
  source: /note=“Description of Artificial Sequence Synthetic polynucleotide”
actagtaccaccatggcttgggtgtggaccttgctattcctgatggcagctgcccaaagtatccaagcacaggttcagttggtgcagtctggatctgagctgaagaagcctggagcctcagtcaaggtttctgcaaggcttctggttataccttcacagactattcaatgcactgggtgcgacaggctccaggacaaggtttaaagtggatgggctggataaacactgagactggtgagccaacatatgcagatgacttagggacggtttgtcttctctttggacacctctgtcagcactgcctatttgcagatcagcagcctcaaagctgaggacacggctgtgtattactgtgctaatccctactatgattacgtctcttactatgctatggactactggggtcagggaaccacggtcaccgtctcctcaggtaagaatggcctctcaagctt

SEQ ID NO: 227 - protein, 107 aa, Artificial Sequence
  source: /note=“Description of Artificial Sequence Synthetic polypeptide”
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 228 - DNA, 415 bp, Artificial Sequence
  source: /note=“Description of Artificial Sequence Synthetic polynucleotide”
gctagcaccaccatggagtcacagattcaggtctttgtattcgtgtttctctggttgtctggtgttgacggagacattcagatgacccagtctccatcctccctgtccgcatcagtgggagacagggtcaccatcacctgcaaggccagtcaggatgtgagtactgctgtagcctggtatcaacagaaaccaggaaaagccctaaactactgatttactcggcatcctacctctacactggagtcccttcacgcttcagtggcagtggatctgggacggatttcactttcaccatcagcagtctgcagcctgaagacattgcaacatattactgtcagcaacattatagtactcctcggacgttcggtcagggcaccaagctggaaatcaaacgtaagtagaatccaaagaattc

SEQ ID NO: 229 - protein, 122 aa, Artificial Sequence
  source: /note=“Description of Artificial Sequence Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 230 - protein, 107 aa, Artificial Sequence
  source: /note=“Description of Artificial Sequence Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 231 - protein, 5 aa, Mus musculus
SHDMS

SEQ ID NO: 232 - protein, 16 aa, Mus musculus
AINSDGGSTYYPDTME

SEQ ID NO: 233 - protein, 11 aa, Mus musculus
HYDDYYAWFAY

SEQ ID NO: 234 - protein, 15 aa, Mus musculus
RASKSVSTSGYSYMH

SEQ ID NO: 235 - protein, 7 aa, Mus musculus
LASNLES

SEQ ID NO: 236 - protein, 9 aa, Mus musculus
QHSRELPLT

SEQ ID NO: 237 - protein, 120 aa, Artificial Sequence
  source: /note=“Description of Artificial Sequence Synthetic polypeptide”
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 238 - DNA, 450 bp, Artificial Sequence
  source: /note=“Description of Artificial Sequence Synthetic polynucleotide”
actagtaccaccatggacttcgggctcagcttggttttccttgtccttattttaaaaagtgtacagtgtgaggtgcagctggtggagtctgggggaggcttagtgcagcctggagggtccctgagactctctgtgcagcctctgaatacgagttcccttcccatgacatgtcttgggtccgccaggctccggggaaggggctggagttggtcgcagccattaatagtgatggtggtagcacctactatccagacaccatggagagacgattcaccatctccagagacaatgccaagaactcactgtacctgcaaatgaacagtctgagggccgaggacacagccgtgtattactgtgcaagacactatgatgattactacgcctggtttgcttactggggccaagggactatggtcactgtctcttcaggtgagtcctaacttcaagctt

SEQ ID NO: 239 - protein, 111 aa, Artificial Sequence
  source: /note=“Description of Artificial Sequence Synthetic polypeptide”
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 240 - DNA, 428 bp, Artificial Sequence
  source: /note=“Description of Artificial Sequence Synthetic polynucleotide”
gctagcaccaccatggagacagacacactcctgttatgggtactgctgctctgggttccaggttccactggtgaaattgtgctgacacagtctcctgctaccttatctttgtctccaggggaaagggccaccctctcatgcagggccagcaaaagtgtcagtacatctggctatagttatatgcactggtaccaacagaaaccaggacaggctcccagactcctcatctatcttgcatccaacctagaatctggggtccctgccaggttcagtggcagtgggtctgggacagacttcaccctcaccatcagcagcctagagcctgaggattttgcagtttattactgtcagcacagtagggagcttccgctcacgttcggcggagggaccaaggtcgagatcaaacgtaagtacacttttctgaattc

SEQ ID NO: 241 - protein, 120 aa, Artificial Sequence
  source: /note=“Description of Artificial Sequence Synthetic polypeptide”
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 242 - protein, 111 aa, Artificial Sequence
  source: /note=“Description of Artificial Sequence Synthetic polypeptide”
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 243 - protein, 452 aa, Artificial Sequence
  source: /note=“Description of Artificial Sequence Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 214 aa, Artificial Sequence
  source: /note=“Description of Artificial Sequence Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC